(19) United States
(12) Patent Application Publication
(10) Pub. No.: US 2023/0204585 A1
(43) Pub. Date: Jun. 29, 2023

(54) HISTOCHEMICAL SYSTEMS AND METHODS FOR EVALUATING EGFR AND EGFR LIGAND EXPRESSION IN TUMOR SAMPLES

(71) Applicant: Ventana Medical Systems, Inc., Tucson, AZ (US)

(72) Inventors: Michael Barnes, San Francisco, CA (US); Joerg Bredno, San Francisco, CA (US); Brian D. Kelly, Tucson, AZ (US); Jim F. Martin, Mountain View, CA (US); Andrea Muranyi, Tucson, AZ (US); Carlos T. Pineda, Tucson, AZ (US); Kandavel Shanmugam, Oro Valley, AZ (US)

(21) Appl. No.: 18/050,425

(22) Filed: Oct. 27, 2022

Related U.S. Application Data
(63) Continuation of application No. PCT/EP2021/061778, filed on May 5, 2021.
(60) Provisional application No. 63/021,627, filed on May 7, 2020.

(51) Int. Cl. G01N 33/574; G01N 33/74; G06T 7/00; G06V 10/25; G06V 10/771

(52) U.S. Cl. CPC G01N 33/57419; G01N 33/5748; G01N 33/74; G06T 7/0012; G06V 10/25; G06V 10/771; G01N 2333/71; G06T 2207/10056; G06T 2207/30096

(57) ABSTRACT

Methods and systems for predictive measures of anti-EGFR therapy response in wild type RAS/EGFR+ samples, e.g., histochemical staining methods for staining EGFR, AREG, and EREG, digital analysis of stained slides, and scoring algorithms that allow prediction of a response to anti-EGFR therapies. Analysis of the stained slides and scoring algorithms may include but are not limited to: a percent tumor cell positivity, computerized clustering algorithms, area density (e.g., area of tumor positive for one or more markers over total tumor area), average intensity (e.g., computerized methodology measuring average gray scale pixel intensity), average intensity broken down according to membrane, cytoplasmic, or punctate staining patterns), or any other appropriate parameter or combination of parameters. The methods of the present invention allow for resolving spatial expression patterns of the ligands and the receptor to determine what patterns are predictive for response to anti-EGFR therapies.

### CROSS-REFERENCE TO RELATED APPLICATIONS

This application is a continuation of International Application PCT/EP2021/061778, filed May 5, 2021, which claims priority to and the benefit of U.S. Provisional Patent Application Ser. No. 63/021,627, filed May 7, 2020.

### SEQUENCE LISTING INCORPORATION BY REFERENCE

This application hereby incorporates-by-reference a sequence listing XML file submitted via the USPTO electronic filing system, having a file name of P34457US1_SeqList, created Oct. 21, 2022, which is 8,263 bytes in size.

### FIELD OF THE INVENTION

The present invention relates to histochemical methods, systems, and compositions for evaluating human Epidermal growth factor receptor (EGFR) protein expression and human EGFR ligand protein expression in colorectal tumors.

### BACKGROUND OF THE INVENTION

About 20% of patients with colon cancer present with metastatic colorectal cancer (mCRC). More than half (50-60%) of these patients will eventually develop incurable advanced disease, which has a 5 year survival rate of approximately 12.5%. Two signaling pathways in mCRC have been the focus of therapeutic drug development: the vascular endothelial growth factor receptor (VEGFR) and the epidermal growth factor receptor (EGFR) pathways. Currently, the majority of the patients with mCRC receive cytotoxic chemotherapy combined with either EGFR or VEGF-targeted therapies. EGFR is overexpressed in about 70% of CRC cases where it is associated with poor outcome. Targeted inhibition of EGFR with monoclonal antibodies, such as cetuximab or panitumumab, was approved by FDA in 2004 and 2006 to treat patients with mCRC. These antibodies target the extracellular domain of EGFR and compete with endogenous ligands to prevent activation of the receptor. By inhibiting EGFR signaling pathway these biological agents inhibit cell proliferation, differentiation, migration and metastasis. Both drugs have very similar efficacy with a 10-15% response rate.

A reliable positive predictor of responsiveness to EGFR-directed therapies has been lacking for some time.

Clinical studies have demonstrated that EGFR inhibitors are the most effective in patients lacking RAS pathway mutations. Point mutations in members of the RAS signaling pathways such as KRAS, NRAS, and BRAF lead to continuous activation of downstream RAS-MAPK signaling, regardless of whether the EGFR pharmacologically inactivated. In addition to RAS and BRAF mutations, other alternative mechanisms such as cMET or EGFR amplification play a role in resistance to cetuximab or panitumumab. Mutation in PI3K or PTEN loss (which often occur with RAS or BRAF mutations) may also be associated with a lack of response. Indeed, RAS, BRAF, and PI3K mutations account for more than 60% of patients with mCRC that show de novo resistance to EGFR-targeted monoclonal antibodies. Of the 40% of patients with KRAS, NRAS, BRAF and PI3K wild type tumors (quadruple wild type patients), approximately half of these patients (only 15%) benefit from anti-EGFR therapy, and more than 20% are non-responders. See Perkins et al., Pharmacogenetics, Vol. 15, Issue 7, pp. 1043-52 (2014).

Over-expression of EGFR ligands—including the ligands epiregulin (EREG) and amphiregulin (AREG)—has been suggested as a predictor for anti-EGFR therapy. In one study of patients with mCRC, addition of anti-EGFR therapy increased survival from 5.1 to 9.8 months in patients having high EREG expression levels compared to the best supportive care alone. This result suggests that EGFR ligands expression might become a clinically useful biomarker to screen patients with mCRC for EGFR inhibitor therapy. However, PCR-based detection systems cannot identify spatial relationships between the ligands and receptors.

Immunohistochemical analysis of EGFR ligands has met with mixed results. Khelwatty et al. (Oncotarget. 2017 Jan. 31; 8(5): 7666-7677), for example, that co-expression of wild type EGFR and at least one of its ligands (at a cutoff of >5% EGFR positive tumor cells and 2+ staining intensity for the ligand) significantly correlates for a shorter progression-free survival, and thus a lower response rate to EGFR-directed therapy. However, in their samples, EGFR staining was predominantly cytoplasmic, which led them to and theorize that internalization of EGFR makes it unavailable for the EGFR therapy to assert antibody-dependent cell-mediated cytotoxicity (ADCC). They further noted that up to 40% of the patients in the study may have previously received cetuximab therapy, which may have contributed to downregulation of EGFR from the surface. Khelwatty therefore does not describe a clear correlation between expression patterns of EGFR and EGFR ligand and response to EGFR-directed therapeutics. Yoshida et al. (Journal of Cancer Research and Clinical Oncology, March 2013, Volume 139. Issue 3, pp 367-378), on the other hand, found good correlation between 4 of the 7 ligands (AREG, HB-EGF, TGFα, and EREG) and clinical response to EGFR therapies, and that response rate was significantly higher in patients expressing 2 or more of the 4 ligands. Yoshida failed, however, to consider any relationship between the expression pattern of EGFR and the EGER ligands. Yoshida therefore is unlikely to completely account for variables that may affect the efficacy of EGFR-directed therapies.

### SUMMARY OF THE INVENTION

This disclosure relates generally to methods, systems, and compositions for the histochemical staining and evaluation of colorectal tumor samples for EGFR and EGFR ligand expression. The disclosed methods, systems, and compositions, are useful for, among other things, stratifying patients according to a predicted response to anti-EGFR therapies and/or for screening colorectal polyps for likelihood of progression to a colorectal cancer.

In an embodiment, a simplex staining methodology is provided, wherein a set of stained sections of a colorectal tumor of a subject are obtained, the set comprising (a1) a first section histochemically stained for a human EGFR protein, and (a2) at least a second section histochemically stained for one or more human EGFR ligand(s), including human AREG protein and/or human EREG protein. The stained sections may be evaluated for expression patterns that correlate with the likelihood that the tumor will respond to an anti-EGFR therapy (such as a therapeutic agent that disrupts association between EGFR and EGFR ligands). In an embodiment, digital images of the sections are obtained and evaluated by a digital pathology methodology comprising registering a digital image of the second section(s) to a digital image of the first section (or vice versa) and then evaluating a spatial relationship between the human EGFR protein and the EGFR ligand(s). If the expression pattern of—and/or the spatial relationship between—the human EGFR protein and the human EGFR ligand(s) is indicative of a tumor that is likely to respond to an anti-EGFR therapy, the subject may be treated with a therapeutic course comprising the anti-EGFR therapy.

In another embodiment, a multiplex methodology is provided, wherein an individual histochemically stained section of a colorectal tumor of a subject is obtained, the individual section being differentially stained for each of (a1) a human EGFR protein, and (a2) at least one of a human AREG protein and a human EREG protein. The stained sections may be evaluated for expression patterns that correlate with the likelihood that the tumor will respond to an anti-EGFR therapy (such as a therapeutic agent that disrupts association between EGFR and EGFR ligands). In an embodiment, digital images of the sections are obtained and evaluated by a digital pathology methodology comprising evaluating an expression pattern of—and/or a spatial relationship between—the human EGFR protein and the EGFR ligand(s). If the expression pattern of—and/or the spatial relationship between—the human EGFR protein and the human EGFR ligand(s) is indicative of a tumor that is likely to respond to an anti-EGFR therapy, the subject may be treated with a therapeutic course comprising the anti-EGFR therapy.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 illustrates two different methods of calculating feature metrics for ROIs. Dashed line in the images illustrates the boundary of an ROI. “X”s in the image indicate objects of interest marked in the image. Circles in the image are control regions that may be used to calculate global metrics for the control region.

FIG. 2A shows the distribution of EREG and AREG mRNA expression (qPCR values) of a cohort.

FIG. 2B shows the expression of EREG mRNA is closely related to the expression of AREG mRNA.

FIG. 3A shows percent of IHC-positive tumor cells compared to qPCR for EREG. The percent positivity correlates well to qPCR for EREG.

FIG. 3B shows percent of IHC-positive tumor cells compared to qPCR for AREG. The percent positivity correlates well to qPCR for AREG.

FIG. 4A-4H show correlations of multiple parameters with qPCR values. FIG. 4A shows percent of IHC-positive cells in Parameter 1 compared to qPCR of EREG. FIG. 4B shows percent of IHC-positive cells in Parameter 1 compared to qPCR of AREG. FIG. 4C shows percent of IHC-positive cells in Parameter 2 compared to qPCR of EREG. FIG. 4D shows percent of IHC-positive cells in Parameter 2 compared to qPCR of AREG. FIG. 4E shows percent of IHC-positive cells in Parameter 3 compared to qPCR of EREG. FIG. 4F shows percent of IHC-positive cells in Parameter 3 compared to qPCR of AREG. FIG. 4G shows percent of IHC-positive cells in Parameter 4 compared to qPCR of EREG. FIG. 4H shows percent of IHC-positive cells in Parameter 4 compared to qPCR of AREG.

FIG. 5A shows membrane stain intensity compared to qPCR of EREG.

FIG. 5B shows membrane stain intensity compared to qPCR of AREG.

FIG. 5C shows cytoplasmic stain intensity compared to qPCR of EREG.

FIG. 5D shows cytoplasmic stain intensity compared to qPCR of AREG.

FIG. 5E shows granular/punctate stain intensity compared to qPCR of EREG.

FIG. 5F shows granular/punctate stain intensity compared to qPCR of AREG.

FIG. 6A, FIG. 6B, and FIG. 6C show an example of a field of view of a stained tissue section. The methods of the present invention may identify every tumor cell and classify it as being marker-negative (displayed in green and blue) or marker-positive (displayed in yellow, orange, red, and magenta). The number of tumor cells on the whole slide may be reported separate for marker-negative and marker-positive cells.

FIG. 7 shows staining of two colorectal cases using a multiplex IHC assay targeting EGFR, Epiregulin (EREG), and Amphiregulin (AREG). In this example, EGFR is stained with DISCOVERY Yellow, EREG is stained with DISCOVERY Teal, and AREG is stained with DISCOVERY Purple.

FIG. 8 shows analysis of the multiplex stained samples using digital pathology. The first row shows that the multiplex matches the signals of the corresponding DAB simplex assays. The second row shows that the multiplex assay is capable of being deconstructed into its constituent stains using digital image analysis. The third row shows deconstructed channels can be recombined and re-colored in order to create a pseudo-DAB image.

### DETAILED DESCRIPTION OF THE INVENTION

This disclosure relates generally to methods, systems, and compositions for the histochemical staining and evaluation of colorectal tumor samples for EGFR and EGFR ligand expression. The disclosed methods, systems, and compositions, are useful for, among other things, stratifying colorectal cancer patients according to a likelihood that their tumor will respond to an EGFR-directed therapy.

### I. TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise.

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, the disclosures of which are incorporated in their entirety herein by reference.

All publications, patent applications, patents, and other references mentioned herein are incorporated by reference in their entirety for all purposes. In case of conflict, the present specification, including explanations of terms, will control.

In order to facilitate review of the various embodiments of the disclosure, the following explanations of specific terms are provided:

Administration: To provide or give a subject an agent, for example, a composition, drug, etc., by any effective route. Exemplary routes of administration include, but are not limited to, oral, injection (such as subcutaneous, intramuscular, intradermal, intraperitoneal, and intravenous), sublingual, rectal, transdermal (e.g., topical), intranasal, vaginal and inhalation routes.

Antibody: A peptide (e.g., polypeptide) that includes at least a light chain or heavy chain immunoglobulin variable region and specifically binds an epitope of an antigen. Antibodies include monoclonal antibodies, polyclonal antibodies, or fragments of antibodies.

Antibody fragment: A molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

Biomarker: As used herein, the term “biomarker” shall refer to any molecule or group of molecules found in a sample that can be used to characterize the sample or a subject from which the sample is obtained. For example, a biomarker may be a molecule or group of molecules whose presence, absence, or relative abundance is: characteristic of a particular disease state; indicative of the severity of a disease or the likelihood or disease progression or regression; and/or predictive that a pathological condition will respond to a particular treatment.

Biomarker-specific reagent: A specific binding agent that is capable of specifically binding directly to one or more biomarkers in the cellular sample or tissue sample. The phrase “[TARGET] biomarker-specific reagent” shall refer to a biomarker-specific reagent that is capable of specifically binding to the recited target biomarker.

Counterstaining: The staining of tissue sections with dyes that allow one to see the entire “landscape” of the tissue section and serve as a reference for the main color used for the detection of tissue targets. Such dyes can stain cell nuclei, the cell membrane, or the entire cell. Examples of dyes include DAPI, which binds to nuclear DNA and emits strong blue light; Hoechst blue stain, which binds to nuclear DNA and emits strong blue light; and Propidium iodide, which binds to nuclear DNA and emits strong red light. Counterstaining of the intracellular cytoskeletal network can be done using phalloidin conjugated to fluorescent dyes. Phalloidin is a toxin that tightly binds to actin filaments in a cell's cytoplasm, which then become clearly visible under the microscope.

Detectable moiety: A molecule or material that can produce a detectable signal (such as a visual, electrical, or other signal) that indicates the presence and/or concentration of the detectable moiety or label deposited on the sample. The detectable signal can be generated by any known or yet to be discovered mechanism including absorption, emission and/or scattering of a photon (including radio frequency, microwave frequency, infrared frequency, visible frequency and ultra-violet frequency photons). Exemplary detectable moieties include (but are not limited to) chromogenic, fluorescent, phosphorescent, and luminescent molecules and materials, catalysts (such as enzymes) that convert one substance into another substance to provide a detectable difference (such as by converting a colorless substance into a colored substance or vice versa, or by producing a precipitate or increasing sample turbidity). In some examples, the detectable moiety is a fluorophore, which belongs to several common chemical classes including coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, Oreg., ThermoFisher Scientific, 11th Edition. In other embodiments, the detectable moiety is a molecule detectable via brightfield microscopy, such as dyes including diaminobenzidine (DAB), 4-(dimethylamino) azobenzene-4′-sulfonamide (DABSYL), tetramethylrhodamine (DISCOVERY Purple), N,N′-biscarboxypentyl-5,5′-disulfonato-indo-dicarbocyanine (Cy5), and Rhodamine 110 (Rhodamine).

Detection reagent: Any reagent used to deposit a detectable moiety in proximity to a biomarker-specific reagent bound to a biomarker in a cellular sample to thereby stain the sample. Non-limiting examples include secondary detection reagents (such as secondary antibodies capable of binding to a primary antibody, anything that specifically binds biotin or avidin), tertiary detection reagents (such as tertiary antibodies capable of binding to secondary antibodies), enzymes directly or indirectly associated with the specific binding agent, chemicals reactive with such enzymes to effect deposition of a fluorescent or chromogenic stain, wash reagents used between staining steps, and the like.

Monoclonal antibody: An antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to a polyclonal antibody, each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method.

Multiplex, -ed, -ing: Staining a single cellular sample with more than one specific binding agent in a manner that the different specific binding agents are differentially detectable.

Polyclonal antibody: An antibody preparation that typically includes different antibodies directed against different determinants (epitopes).

Sample: Any material obtained for a diagnostic purpose from a subject and processed in a manner compatible with testing for the presence or absence and/or the amount of a biomarker in the material using a specific binding agent. Examples of diagnostic purposes include: diagnosing or prognosing disease in the subject, and/or predicting response of a disease to a particular therapeutic regimen, and/or monitoring a subject's response to a therapeutic regimen, and/or monitoring for progression or recurrence of disease.
(a) Cellular sample: A sample containing intact cells, such as cell cultures, blood or other body fluid samples containing cells, cell smears (such as Pap smears and cervical monolayers), fine needle aspirates (FNA), liquid based cytology samples, and surgical specimens taken for pathological, histological, or cytological interpretation.
(b) Tissue sample: A cellular sample that preserves the cross-sectional spatial relationship between the cells as they existed within the subject from which the sample was obtained. “Tissue sample” shall encompass both primary tissue samples (i.e. cells and tissues produced by the subject) and xenografts (i.e. foreign cellular samples implanted into a subject).

Section: When used as a noun, a thin slice of a tissue sample suitable for microscopic analysis, typically cut using a microtome. When used as a verb, making a section of a tissue sample, typically using a microtome.

Serial Section: Any one of a series of sections cut in sequence from a tissue sample. For two sections to be considered “serial sections” of one another, they do not necessarily need to be consecutive sections from the tissue, but they should generally contain the same tissue structures in the same cross-sectional relationship, such that the structures can be matched to one another after histological staining.

Specific Binding: As used herein, the phrase “specific binding,” “specifically binds to,” or “specific for” refers to measurable and reproducible interactions such as binding between a target and a specific binding agent, which is determinative of the presence of the target in the presence of a heterogeneous population of molecules including biological molecules. For example, a binding entity that specifically binds to a target may be an antibody that binds the target with greater affinity, avidity, more readily, and/or with greater duration than it binds to other targets.

Specific binding agent: Any composition of matter that is capable of specifically binding to a target chemical structure associated with a cellular sample or tissue sample (such as a biomarker expressed by the sample or a biomarker-specific reagent bound to the sample). Examples include but are not limited to nucleic acid probes specific for particular nucleotide sequences; antibodies and antigen binding fragments thereof; and engineered specific binding structures, including ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, Calif.), dAbs (scaffold based on VH or VL antibody domain; GlaxoSmithKline PLC, Cambridge, UK), DARPins (scaffold based on Ankyrin repeat proteins; Molecular Partners AG, Zurich, CH), ANTICALINs (scaffold based on lipocalins; Pieris AG, Freising, DE), NANOBODYs (scaffold based on VHH (camelid Ig); Ablynx N/V, Ghent, BE), TRANS-BODYs (scaffold based on Transferrin; Pfizer Inc., New York, N.Y.), SMIPs (Emergent Biosolutions, Inc., Rockville, Md.), and TETRANECTINs (scaffold based on C-type lectin domain (CTLD), tetranectin; Borean Pharma A/S, Aarhus, DK). Descriptions of such engineered specific binding structures are reviewed by Wurch et al., Development of Novel Protein Scaffolds as Alternatives to Whole Antibodies for Imaging and Therapy: Status on DISCOVERY Research and Clinical Validation, Current Pharmaceutical Biotechnology, Vol. 9, pp. 502-509 (2008), the content of which is incorporated by reference.

Stain: When used as a noun, the term “stain” shall refer to any substance that can be used to visualize specific molecules or structures in a cellular sample for microscopic analysis, including brightfield microscopy, fluorescent microscopy, electron microscopy, and the like. When used as a verb, the term “stain” shall refer to any process that results in deposition of a stain on a cellular sample.

Subject: A mammal from which a sample has been obtained or derived. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject is a human.

### II. HISTOCHEMICAL METHODS FOR LABELING COLORECTAL SAMPLES FOR EGFR AND EGFR LIGANDS

The present methods, systems, and compositions are based on staining colorectal tumor samples for EGFR protein and one or more of EREG and AREG.

In an embodiment, staining of the colorectal tumor samples is performed by a simplex method. A simplex histochemical stain is a staining method in which a single biomarker-specific reagents (or group of biomarker-specific reagents) is applied to a single section and stained with a single color stain. Simplex methods allow the user to avoid complicated multiplex staining processes and analytical methods. Where a spatial relationship between the different biomarkers is important, digital analysis comprising registration of the stained images to one another may be used.

In an embodiment, a simplex histochemical staining method is provided, wherein the simplex method results in at least the following set of stained colorectal tumor samples: (a) a first sample derived from a colorectal tumor, wherein the first sample is histochemically stained for human EGFR protein; and (b) a second sample derived from the same colorectal tumor as the first sample, wherein the second sample is histochemically stained for one or more of human EREG protein and human AREG protein. In an embodiment, the set of stained colorectal tumor samples comprises: (a) a first sample derived from a colorectal tumor, wherein the first sample is histochemically stained for human EGFR protein; and (b) a second sample derived from the same colorectal tumor as the first sample, wherein the second sample is histochemically stained for human EREG protein. In an embodiment, the set of stained colorectal tumor samples comprises: (a) a first sample derived from a colorectal tumor, wherein the first sample is histochemically stained for human EGFR protein; and (b) a second sample derived from the same colorectal tumor as the first sample, wherein the second sample is histochemically stained for human AREG protein. In an embodiment, the set of stained colorectal tumor samples comprises: (a) a first sample derived from a colorectal tumor, wherein the first sample is histochemically stained for human EGFR protein; (b) a second sample derived from the same colorectal tumor as the first sample, wherein the second sample is histochemically stained for human EREG protein; and (c) a third sample derived from the same colorectal tumor as the first sample, wherein the third sample is histochemically stained for human AREG protein. In an embodiment, the set of stained colorectal tumor samples comprises: (a) a first sample derived from a colorectal tumor, wherein the first sample is histochemically stained for human EGFR protein; and (b) a second sample derived from the same colorectal tumor as the first sample, wherein the second sample is histochemically stained for human AREG protein and for human EREG protein. In an embodiment, the first, second, and/or third samples are tissue sections from the same fixed tissue sample. In an embodiment, the sections are made from a formalin-fixed, paraffin-embedded (FFPE) tissue sample. In an embodiment, the first, second, and/or third samples are serial sections from the same FFPE tissue sample. In an embodiment, a set of stained serial sections are provided, the set of stained serial sections comprising: (a) the first, second, and/or third serial sections. In another embodiment, the set of stained serial sections may further comprise: (b) an additional serial section stained with a morphological stain (such as hematoxylin and eosin (H&E)).

In an embodiment, staining of the colorectal tumor samples is performed by a multiplex method. A multiplex histochemical stain is a staining method in which multiple biomarker-specific reagents are applied to a single section and stained with dyes that are distinguishable from one another. In multiplex staining methods, the biomarker-specific reagents and detection reagents are applied in a manner that allows the different biomarkers to be differentially labeled. Multiplex methods allow the user to observe spatial relationships between the different biomarkers without having to resort to registration of separate histochemically-stained slides to one another.

In an embodiment, a multiplex histochemical staining method is provided, wherein the multiplex method results in stained colorectal tumor samples derived from a colorectal tumor, wherein the stained colorectal sample is histochemically stained for human EGFR protein and one or more of human EREG protein and human AREG protein, wherein the histochemical stain for human EREG protein is distinguishable from the histochemical stain for the one or more of human EREG protein and human AREG protein. In an embodiment, the stained colorectal sample is histochemically stained for human EGFR protein and human EREG protein. In an embodiment, the stained colorectal sample is histochemically stained for human EGFR protein and human EREG protein, wherein the histochemical stain for EGFR is distinguishable from the histochemical stain for EREG. In an embodiment, the stained colorectal sample is histochemically stained for human EGFR protein and human AREG protein, wherein the histochemical stain for EGFR is distinguishable from the histochemical stain for AREG. In an embodiment, the stained colorectal sample is histochemically stained for human EGFR protein, human EREG protein, and human AREG protein, wherein the histochemical stain for EGFR is distinguishable from the histochemical stain for EREG, and wherein the histochemical stain for AREG is distinguishable from the histochemical stain for EGFR and the histochemical stain for EREG. In an embodiment, the stained colorectal sample is histochemically stained for human EGFR protein, human EREG protein, and human AREG protein, wherein the histochemical stain for EGFR is distinguishable from the histochemical stain for EREG, and wherein the histochemical stain for AREG is not distinguishable from the histochemical stain for EREG.

A. Samples and Sample Preparation

The present methods are performed on tissue samples of colorectal tissue obtained from subjects suspected of having a colorectal tumor, including, for example, tumor biopsies samples and resection samples.

In an embodiment, the tissue sample is a fixed tissue sample. Fixing a tissue sample preserves cells and tissue constituents in as close to a life-like state as possible and allows them to undergo preparative procedures without significant change. Autolysis and bacterial decomposition processes that begin upon cell death are arrested, and the cellular and tissue constituents of the sample are stabilized so that they withstand the subsequent stages of tissue processing. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum. The most commonly used fixative in preparing samples is formaldehyde, generally in the form of a formalin solution (formaldehyde in an aqueous (and typically buffered) solution). In an embodiment, the samples used in the present methods are fixed by a method comprising fixation in a formalin-based fixative. In one example, the fixative is 10% neutral buffered formalin. Notwithstanding these examples, the tissues can be fixed by process using any fixation medium that is compatible with the biomarker-specific reagents and specific detection reagents used.

In some examples, the fixed tissue sample is embedded in an embedding medium. An embedding medium is an inert material in which tissues and/or cells are embedded to help preserve them for future analysis. Embedding also enables tissue samples to be sliced into thin sections. Embedding media include paraffin, celloidin, OCT™ compound, agar, plastics, or acrylics. In an embodiment, the sample is fixed in formalin and embedded in paraffin to form a formalin-fixed, paraffin-embedded (FFPE) block. In a typical embedding process (such as used for FFPE blocks), after the sample is fixed it is subjected to a series of alcohol immersions, typically using increasing alcohol concentrations ranging from about 70% to about 100%, to dehydrate the sample. The alcohol generally is an alkanol, particularly methanol and/or ethanol. Particular working embodiments have used 70%, 95% and 100% ethanol for these serial dehydration steps. After the last alcohol treatment step the sample is then immersed into another organic solvent, commonly referred to as a clearing solution. The clearing solution (1) removes residual alcohol, and (2) renders the sample more hydrophobic for a subsequent waxing step. The clearing solvent typically is an aromatic organic solvent, such as xylene. Blocks are formed by applying the embedding material to the cleared sample, from which tissue sections can be cut (such as by using a microtome).

Notwithstanding these examples, no specific processing step is required by the present disclosure, so long as the tissue sample obtained is compatible with histochemical staining of the sample for the biomarkers of interest and the reagents used for that staining and subsequent microscopic evaluation or digital imaging.

B. Sample Selection

In an embodiment, the tumor from which the sample is derived is staged prior to being stained for EGFR protein and EREG and/or AREG protein(s). Stage 0 colorectal cancers are cancers that have not grown beyond the inner lining of the colon. Stage I colorectal cancers are cancers that have not spread outside of the colon wall itself or into nearby lymph nodes. Stage II colorectal cancers are cancers that have grown through the wall of the colon, and possibly into nearby tissue, but have not yet spread to the lymph nodes. Stage III colorectal cancers are cancers that have spread to nearby lymph nodes, but have not yet spread to other parts of the body. Stage IV colorectal cancers are cancers that have spread from the colon to distant organs and tissues. In an embodiment, the sample is selected for staining if it is a stage III or a stage IV colorectal cancer. In another embodiment, the sample is selected for staining if it is a stage IV colorectal cancer.

C. Histochemical Staining, Generally

Labeling of a target biomarker may be accomplished by contacting a tissue section with a biomarker-specific reagent under conditions that facilitate specific binding between the target biomarker and the biomarker-specific reagent. The sample is then contacted with a set of detection reagents that interact with the biomarker-specific reagent to facilitate deposition a detectable moiety in close proximity the target biomarker on the sample, thereby generating a detectable signal localized to the target biomarker. Biomarker-stained sections may optionally be additionally stained with a contrast agent (such as a hematoxylin stain) to visualize macromolecular structures. Additionally, a serial section of the biomarker-stained section or the biomarker-stained section may be stained with a morphological stain, which can help with identification of regions of interest for subsequent digital analysis.

The labeling methods herein may be performed on an automated staining machine (or other slide processing machine), manually, or feature a combination of automated steps and manual steps.

C1. Biomarker-Specific Reagents

The histochemical staining methods disclosed herein comprise contacting a tissue section of a colorectal tumor with one or more under biomarker-specific reagents for human EGFR protein, human EREG protein, and/or human AREG protein under conditions that support specific binding between biomarker-specific reagents and the biomarkers expressed by the sample. EREG and AREG—like all EGFR ligands—are expressed first as a pro-peptide, which is cleaved at the cell surface to release an active signaling domain. Canonical amino acid sequences for full length human EGFR, and human EREG and AREG (and pro-peptides thereof) are set forth in Table 1. As would be understood by a person of ordinary skill in the art, the precise amino acid sequences may vary slightly from subject-to-subject.

TABLE 1

| BIOMARKER | UNIPROT ID | SEQ ID NO |
|---|---|---|
| Epidermal Growth Factor Receptor | P00533-1 | 1 |
| Proepiregulin (including signal peptide) | O14944-1 | 2 |
| Mature Epiregulin | O14944-1 | 2 (aa 63-108) |
| Amphiregulin Pro-peptide | P15514-1 | 3 |
| Mature Amphiregulin | P15514-1 | 3 (aa 101-187) |

In an embodiment, the biomarker-specific reagent to human EGFR protein is a biomarker-specific reagent capable of specifically binding to a polypeptide comprising SEQ ID NO: 1. In an embodiment, the biomarker-specific reagent to human EREG protein is a biomarker-specific reagent capable of specifically binding to a polypeptide comprising SEQ ID NO: 2. In an embodiment, the biomarker-specific reagent to human AREG protein is a biomarker-specific reagent capable of specifically binding to a polypeptide comprising SEQ ID NO: 3.

In an embodiment, the EGFR biomarker-specific reagent is an antibody. In another embodiment, the antibody is a monoclonal antibody. Non-limiting examples of an EGFR-specific monoclonal antibodies are set forth in Table 2:

TABLE 2

| Clone | Species | Epitope/Immunogen | Reference | Manufacturer |
|---|---|---|---|---|
| 5B7 | Rabbit | Intracellular epitope located in | Mascaux et al., Clin. | Ventana |
| | | the suppressor of cytokine | Cancer Res., vol. 17, | Medical |
| | | signaling 3 (SOCS3) protein | issue 24, pp. 7796- | Systems, Inc. |
| | | binding site | 7807 (December 2011) | |
| EGFR.25 | Mouse | 200 amino acids of the | Shinojima et al., | Leica |
| | | intracellular domain of the | Cancer Res., Vol. 63, | Biosystems |
| | | EGFR molecule, excluding | Issue 20 (October 2003). | Newcastle Ltd. |
| | | the conserved tyrosine kinase | | |
| | | domain | | |
| 2-18C9 | Mouse | Extracellular epitope located | Spaulding & | Dako |
| | | near ligand binding site and | Spaulding, Seminars | |
| | | conserved in EGFRvIII | in Oncology, Vol. 29, | |
| | | truncated mutants | Issue 5, Supplement | |
| | | | 14, pp. 45-54 (October | |
| | | | 2002) | |
| EGFR.113 | Mouse | Immunogen comprising | Shinojima et al., | Leica |
| | | extracellular domain | Cancer Res., Vol. 63, | Biosystems |
| | | | Issue 20 (October 2003). | Newcastle Ltd. |
| H11 | Mouse | Immunogen is HC2 20 d2 | Anagnostou et al., | Thermo-Fisher |
| | | cells; recognizes epitope | Cancer epidemiology, | Biocare |
| | | located in extracellular | biomarkers & | |
| | | domain and conserved in | prevention, Vol. 19, | |
| | | EGFRvIII truncated mutants | Issue 4, pp. 982-991 | |
| | | | (2010) | |
| 15F8 | Rabbit | Produced by immunizing | Anagnostou et al., | Cell Signaling |
| | | animals with a synthetic | Cancer epidemiology, | Technology, |
| | | peptide corresponding to | biomarkers & | Inc. |
| | | residues near the carboxy | prevention, Vol. 19, | |
| | | terminus of human EGF | Issue 4, pp. 982-991 | |
| | | receptor. | (2010) | |
| | | | Cell Signaling Product | |
| | | | Data Sheet | |

In an embodiment, the EGFR biomarker-specific reagent is a monoclonal antibody directed against an intracellular domain of EGFR. In another embodiment, the EGFR biomarker-specific reagent is a monoclonal antibody directed against an extracellular domain of EGFR. In another embodiment, the EGFR biomarker-specific reagent is a monoclonal antibody that recognizes both full length EGFR and EGFRvIII mutant.

In an embodiment, the EREG biomarker-specific reagent is an antibody. Non-limiting examples of an EREG-specific antibodies are set forth in Table 3:

TABLE 3

| Clone | Species | Epitope/Immunogen | Reference | Manufacturer |
|---|---|---|---|---|
| J5H1L1 | Rabbit | Immunogen comprising amino acid | WO 2017-001350 A1 | Not |
| | Monoclonal | residues 148-169 of SEQ ID NO: | (incorporated by reference) | commercially |
| | | 2. Does not recognize mature | | available |
| | | EREG. | | |
| J89H12L3 | Rabbit | Immunogen comprising amino acid | WO 2017-001350 A1 | Not |
| | Monoclonal | residues 156-169 of SEQ ID NO: | (incorporated by reference) | commercially |
| | | 2. Does not recognize mature | | available |
| | | EREG. | | |
| J89H12L8 | Rabbit | Immunogen comprising amino acid | WO 2017-001350 A1 | Not |
| | Monoclonal | residues 156-169 of SEQ ID NO: | (incorporated by reference) | commercially |
| | | 2. Does not recognize mature | | available |
| | | EREG. | | |
| D4O5I | Rabbit | Raised against residues surrounding | CST product insert | Cell Signaling |
| | Monoclonal | Glu155. Recognizes endogenous | | Technology, |
| | | levels of proepiregulin and the C- | | Inc. |
| | | terminal propeptide of the EREG | | |
| | | protein. It does not recognize the | | |
| | | mature form of EREG. | | |

In an embodiment, the EREG biomarker-specific reagent is a monoclonal antibody selected from Table 3.

In an embodiment, the AREG biomarker-specific reagent is an antibody. Non-limiting examples of an AREG-specific antibodies are set forth in Table 4:

TABLE 4

| Clone | Species | Epitope/Immunogen | Reference | Manufacturer |
|---|---|---|---|---|
| J111H1L10 | Rabbit | Immunogen comprising amino acid | WO 2017-001350 A1 | Not |
| | Monoclonal | residues 238-252 of SEQ ID NO: | (incorporated by reference) | commercially |
| | | 3. Does not recognize mature | | available |
| | | AREG. | | |
| G-4 | Mouse | Raised against amino acids 1-155 | Santa Cruz Datasheet | Santa Cruz |
| | monoclonal | of amphiregulin of human origin | | Biotechnology, |
| | | | | Inc. |
| AF262 | Goat | Val107-Lys184 of UNIPROT | R&D Systems Datasheet | R&D Systems |
| | polyclonal | Accession No. P15514 | | |

In an embodiment, the AREG biomarker-specific reagent is selected from Table 4.

C2. Antigen Retrieval

Fixation chemically alters the substituents of the samples. This sometimes alters the ability of the biomarker-specific reagent to specifically bind to its biomarker. In some cases, the effects of fixation can be overcome by treating the sample prior to contacting it with the biomarker-specific reagent, a process commonly referred to as antigen retrieval. Antigen retrieval can be achieved by physical approaches, chemical approaches, or a combination of both. Examples of methods of antigen retrieval are discussed in Shi et al. (J Histochemistry & Cytochemistry, 2011, 59:13-32), D'Amico et al. (J Immunological Methods, 2009, 341:1-18), and McNicoll and Richmond (Histopathology, 1998, 32:97-103), as well we U.S. Pat. Nos. 9,506,928 and 6,544,798. In an example, antigen retrieval may be achieved by treating the sample with proteases (e.g., trypsin, DNase, proteinase K, pepsin, pronase, ficin, etc.) (termed protease-induced epitope retrieval (PIER)). In another example, fixed samples are heated while in contact with buffered solutions (termed heat induced epitope retrieval (HIER)). HIER techniques may be optimized by varying the temperature (for example, up to ˜100° C.), time (typically up to 30 minutes), and/or pH (for example in a range of pH ˜6 to pH ˜10). Exemplary HIER solutions include citrate buffered solutions (for example at pH ˜6), ethylenediaminetetraacetic acid (EDTA) solutions (for example at pH ˜8), tris(hydroxymethyl)aminomethane (Tris)—EDTA buffer (for example at pH ˜9), Tris buffer (for example, pH ˜10), glycine-HCl buffer, periodic acid, urea, lead thiocyanate solutions, etc.

In an embodiment, a simplex method is provided, wherein an antigen retrieval condition is selected and optimized for each set of biomarker-specific reagents applied to each individual tissue section. In another embodiment, a multiplex method is provided, wherein a set of biomarker-specific reagent comprising an EGFR biomarker-specific reagent and one or more of an EREG biomarker-specific reagent and an AREG biomarker-specific reagent, where an antigen retrieval condition for the tissue section to be stained is selected that is compatible with each biomarker-specific reagent of the set.

Notwithstanding these examples, no specific antigen retrieval step is required by the present disclosure, so long as the tissue sample obtained is compatible with histochemical staining of the sample for the biomarkers of interest and the reagents used for that staining and subsequent microscopic evaluation or digital imaging of the stained sample.

C3. Detection Schemes

In the present histochemical methods, the biomarker-specific reagent facilitates detection of the biomarker by mediating deposition of a detectable moiety on the sample in close proximity to the biomarker to which the biomarker-specific reagent is bound.

In an embodiment, the detectable moiety is directly conjugated to the biomarker specific reagent, and thus is deposited on the sample upon binding of the biomarker-specific reagent to its target. Such a detection scheme is referred to as a “direct detection method.”

In other embodiments, deposition of the detectable moiety is effected by contacting a sample to which the biomarker-specific reagent is bound with one or more detection reagents, wherein the detection reagents interact with the biomarker-specific reagent and each other such that a detectable moiety is deposited on the sample near where the biomarker-specific reagent is bound, but not at points distant from where the biomarker-specific reagent is bound. Such a detection scheme is referred to as an “indirect detection method.”

In an embodiment, an indirect detection method is used, wherein the detectable moiety is deposited via an enzymatic reaction localized to the biomarker-specific reagent. Suitable enzymes for such reactions are well known and include, but are not limited to, oxidoreductases, hydrolases, phosphatases, and peroxidases. Specific enzymes explicitly included are horseradish peroxidase (HRP), alkaline phosphatase (AP), acid phosphatase, glucose oxidase, β-galactosidase, β-glucuronidase, and β-lactamase. The enzyme may be directly conjugated to the biomarker-specific reagent, or may be indirectly associated with the biomarker-specific reagent via a labeling conjugate. As used herein, a “labeling conjugate” comprises: (a) a specific binding agent; and (b) an enzyme conjugated to the specific binding agent, wherein the enzyme is reactive with a chromogenic substrate, signaling conjugate, or enzyme-reactive dye under appropriate reaction conditions to effect in situ generation of the dye and/or deposition of the dye on the tissue sample.

In non-limiting examples, the specific binding agent of the labeling conjugate may be a secondary detection reagent (such as a species-specific secondary antibody bound to a primary antibody, an anti-hapten antibody bound to a hapten-conjugated primary antibody, or a biotin-binding protein bound to a biotinylated primary antibody), a tertiary detection reagent (such as a species-specific tertiary antibody bound to a secondary antibody, an anti-hapten antibody bound to a hapten-conjugated secondary antibody, or a biotin-binding protein bound to a biotinylated secondary antibody), or other such arrangements.

A hapten is a molecule, typically a small molecule, which can combine or bind specifically with an antibody, but typically is substantially incapable of being immunogenic except in combination with a carrier molecule. Many haptens are known and frequently used for analytical procedures, such as dinitrophenyl (DNP), biotin, digoxigenin (DIG), fluorescein, rhodamine, or those disclosed in U.S. Pat. No. 7,695,929, the disclosure of which is incorporated in its entirety herein by reference. Other haptens have been specifically developed by Ventana Medical Systems, Inc., assignee of the present application, including haptens selected from oxazoles, pyrazoles, thiazoles, nitroaryls, benzofurans, triterpenes, ureas, thioureas, rotenoids, coumarins, cyclolignans, and combinations thereof, with particular hapten examples of haptens including benzofurazan, nitrophenyl, 4-(2-hydroxyphenyl)-1H-benzo[b][1,4]diazepine-2(3H)-one, and 3-hydroxy-2-quinoxalinecarbamide. Plural different haptens may be coupled to a polymeric carrier. Moreover, compounds, such as haptens, can be coupled to another molecule using a linker, such as an NHS-PEG linker.

An enzyme thus localized to the sample-bound biomarker-specific reagent can then be used in a number of schemes to deposit a detectable moiety.

In some embodiments, the enzyme reacts with a chromogenic compound/substrate. Particular non-limiting examples of chromogenic compounds/substrates include 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue, or tetrazolium violet.

In some embodiments, the enzyme can be used in a metallographic detection scheme. Metallographic detection methods include using an enzyme such as alkaline phosphatase (AP) in combination with a water-soluble metal ion and a redox-inactive substrate of the enzyme. In some embodiments, the substrate is converted to a redox-active agent by the enzyme, and the redox-active agent reduces the metal ion, causing it to form a detectable precipitate (see, for example, U.S. patent application Ser. No. 11/015,646, filed Dec. 20, 2004, PCT Publication No. 2005/003777 and U.S. Patent Application Publication No. 2004/0265922; each of which is incorporated by reference herein in its entirety). Metallographic detection methods may also include using an oxido-reductase enzyme (such as horseradish peroxidase) along with a water soluble metal ion, an oxidizing agent and a reducing agent, to form a detectable precipitate (see, for example, U.S. Pat. No. 6,670,113, which is incorporated by reference herein in its entirety).

In some embodiments, the enzymatic reaction occurs between the enzyme and the dye itself, wherein the reaction converts the dye from a non-binding species to a species deposited on the sample. For example, reaction of DAB with a peroxidase (such as horseradish peroxidase) oxidizes the DAB, causing it to precipitate.

In yet other embodiments, the detectable moiety is deposited via a signaling conjugate comprising a latent reactive moiety configured to react with the enzyme to form a reactive species that can bind to the sample or to other detection components. These reactive species are capable of reacting with the sample proximal to their generation, i.e. near the enzyme, but rapidly convert to a non-reactive species so that the signaling conjugate is not deposited at sites distal from the site at which the enzyme is deposited. Examples of latent reactive moieties include: quinone methide (QM) analogs, such as those described at WO2015124703A1, and tyramide conjugates, such as those described at, WO2012003476A2, each of which is hereby incorporated by reference herein in its entirety. In some examples, the latent reactive moiety is directly conjugated to a dye, such as N,N′-biscarboxypentyl-5,5′-disulfonato-indo-dicarbocyanine (Cy5), 4-(dimethylamino) azobenzene-4′-sulfonamide (DABSYL), tetramethylrhodamine (DISCOVERY Purple, Ventana, Tucson, Ariz.), or Rhodamine 110 (Rhodamine). In other examples, the latent reactive moiety is conjugated to one member of a specific binding pair, and the dye is linked to the other member of the specific binding pair. In other examples, the latent reactive moiety is linked to one member of a specific binding pair, and an enzyme is linked to the other member of the specific binding pair, wherein the enzyme is (a) reactive with a chromogenic substrate to effect generation of the dye, or (b) reactive with a dye to effect deposition of the dye (such as DAB). Examples of specific binding pairs include: (1) a biotin or a biotin derivative (such as desthiobiotin) linked to the latent reactive moiety, and a biotin-binding entity (such as avidin, streptavidin, deglycosylated avidin (such as NEUTRAVIDIN), or a biotin binding protein having a nitrated tyrosine at its biotin binding site (such as CAPTAVIDIN)) linked to a dye or to an enzyme reactive with a chromogenic substrate or reactive with a dye (for example, a peroxidase linked to the biotin-binding protein when the dye is DAB); and (2) a hapten linked to the latent reactive moiety, and an anti-hapten antibody linked to a dye or to an enzyme reactive with a chromogenic substrate or reactive with a dye (for example, a peroxidase linked to the anti-hapten antibody when the dye is DAB).

Non-limiting examples of biomarker-specific reagent and detection reagent combinations set forth in Table 5 are specifically included.

TABLE 5
A. Biomarker-specific reagent linked directly to detectable moiety
Biomarker-specific reagent-Dye conjugate
B. Biomarker-specific reagent linked to enzyme reacting with detectable moiety
Biomarker-specific reagent-Enzyme conjugate + DAB
Biomarker-specific reagent-Enzyme conjugate + Chromogen
C. Biomarker-specific reagent linked to Enzyme reacting with detectable moiety

| | |
|---|---|
| C1. Signaling | Biomarker-specific reagent-Enzyme conjugate + QM-Dye conjugate |
| conjugate comprises | Biomarker-specific reagent-Enzyme conjugate + Tyramide-Dye conjugate |
| detectable moiety | |
| C2. Signaling | Biomarker-specific reagent-Enzyme conjugate + QM-Enzyme conjugate + |
| conjugate comprises | DAB |
| enzyme that reacts | Biomarker-specific reagent-Enzyme conjugate + QM-Enzyme conjugate + |
| directly with | Chromogen |
| detectable moiety | Biomarker-specific reagent-Enzyme conjugate + Tyramide-Enzyme |
| | conjugate + DAB |
| | Biomarker-specific reagent-Enzyme conjugate + Tyramide-Enzyme |
| | conjugate + Chromogen |
| C3. Signaling | Biomarker-specific reagent-Enzyme conjugate + QM-Enzyme conjugate + |
| conjugate comprises | QM-Dye conjugate |
| enzyme that reacts | Biomarker-specific reagent-Enzyme conjugate + QM-Enzyme conjugate + |
| with second signaling | Tyramide-Dye conjugate |
| conjugate comprising | Biomarker-specific reagent-Enzyme conjugate + Tyramide-Enzyme |
| detectable moiety | conjugate + QM-Dye conjugate |
| | Biomarker-specific reagent-Enzyme conjugate + Tyramide-Enzyme |
| | conjugate + Tyramide-Dye conjugate |
| C4. Signaling | Biomarker-specific reagent-Enzyme conjugate + Tyramide-(biotin/hapten) |
| conjugate comprises | conjugate + Dye-(avidin/anti-hapten Biomarker-specific reagent) conjugate |
| member of a specific | Biomarker-specific reagent-Enzyme conjugate + QM-(biotin/hapten) |
| binding pair and other | conjugate + Dye-(avidin/anti-hapten Biomarker-specific reagent) conjugate |
| member of binding | |
| pair is linked to | |
| detectable moiety | |
| C5. Signaling | Biomarker-specific reagent-Enzyme conjugate + QM-(biotin/hapten) |
| conjugate comprises | conjugate + Enzyme-(avidin/anti-hapten Biomarker-specific reagent) |
| member of a specific | conjugate + DAB |
| binding pair and other | Biomarker-specific reagent-Enzyme conjugate + QM-(biotin/hapten) |
| member of binding | conjugate + Enzyme-(avidin/anti-hapten Biomarker-specific reagent) |
| pair is linked to | conjugate + Chromogen |
| enzyme reactive with | Biomarker-specific reagent-Enzyme conjugate + Tyramide-(biotin/hapten) |
| detectable moiety | conjugate + Enzyme-(avidin/anti-hapten Biomarker-specific reagent) |
| | conjugate + DAB |
| | Biomarker-specific reagent-Enzyme conjugate + Tyramide-(biotin/hapten) |
| | conjugate + Enzyme-(avidin/anti-hapten Biomarker-specific reagent) |
| | conjugate + Chromogen |
| C6. Signaling | Biomarker-specific reagent-Enzyme conjugate + QM-(biotin/hapten) |
| conjugate comprises | conjugate + Enzyme-(avidin/anti-hapten Biomarker-specific reagent) |
| member of a specific | conjugate + Tyramide-Dye conjugate |
| binding pair and other | Biomarker-specific reagent-Enzyme conjugate + QM-(biotin/hapten) |
| member of binding | conjugate + Enzyme-(avidin/anti-hapten Biomarker-specific reagent) |
| pair is linked to | conjugate + QM-Dye conjugate |
| enzyme reactive with | Biomarker-specific reagent-Enzyme conjugate + Tyramide-(biotin/hapten) |
| second detectable | conjugate + Enzyme-(avidin/anti-hapten Biomarker-specific reagent) |
| moiety linked to a | conjugate + Tyramide-Dye conjugate |
| detectable moiety | Biomarker-specific reagent-Enzyme conjugate + Tyramide-(biotin/hapten) |
| | conjugate + Enzyme-(avidin/anti-hapten Biomarker-specific reagent) |
| | conjugate + QM-Dye conjugate |

D. Biomarker-specific reagent linked to member of specific binding pair

| | |
|---|---|
| D1. Dye linked to | Biomarker-specific reagent-(biotin/hapten) conjugate + Dye-(avidin/anti- |
| other member of | hapten Biomarker-specific reagent) conjugate |
| specific binding pair | |
| D2. Enzyme linked to | Biomarker-specific reagent-(biotin/hapten) conjugate + Enzyme- |
| other member of | (avidin/anti-hapten Biomarker-specific reagent) conjugate + DAB |
| specific binding pair, | Biomarker-specific reagent-(biotin/hapten) conjugate + Enzyme- |
| wherein the enzyme is | (avidin/anti-hapten Biomarker-specific reagent) conjugate + Chromogen |
| reactive with | Biomarker-specific reagent-(biotin/hapten) conjugate + Enzyme- |
| detectable moiety | (avidin/anti-hapten Biomarker-specific reagent) conjugate + QM-Dye |
| | conjugate |
| | Biomarker-specific reagent-(biotin/hapten) conjugate + Enzyme- |
| | (avidin/anti-hapten Biomarker-specific reagent) conjugate + Tyramide-Dye |
| | conjugate |

E. Secondary detection reagent linked directly to detectable moiety
Biomarker-specific reagent + 2° Biomarker-specific reagent-Dye conjugate
F. Secondary detection reagent linked to Enzyme reacting with detectable moiety
Biomarker-specific reagent + 2° Biomarker-specific reagent-Enzyme conjugate + DAB
Biomarker-specific reagent + 2° Biomarker-specific reagent-Enzyme conjugate + Chromogen
G. Secondary detection reagent linked to Enzyme reacting with detectable moiety

| | |
|---|---|
| G1. Signaling | Biomarker-specific reagent + 2° Biomarker-specific reagent-Enzyme |
| conjugate comprises | conjugate + QM-Dye conjugate |
| detectable moiety | Biomarker-specific reagent + 2° Biomarker-specific reagent-Enzyme |
| | conjugate + Tyramide-Dye conjugate |
| G2. Signaling | Biomarker-specific reagent + 2° Biomarker-specific reagent-Enzyme |
| conjugate comprises | conjugate + QM-Enzyme conjugate + DAB |
| enzyme that reacts | Biomarker-specific reagent + 2° Biomarker-specific reagent-Enzyme |
| directly with | conjugate + QM-Enzyme conjugate + Chromogen |
| detectable moiety | Biomarker-specific reagent + 2° Biomarker-specific reagent-Enzyme |
| | conjugate + Tyramide-Enzyme conjugate + DAB |
| | Biomarker-specific reagent + 2° Biomarker-specific reagent-Enzyme |
| | conjugate + Tyramide-Enzyme conjugate + Chromogen |
| G3. Signaling | Biomarker-specific reagent + 2° Biomarker-specific reagent-Enzyme |
| conjugate comprises | conjugate + QM-Enzyme conjugate + QM-Dye conjugate |
| enzyme that reacts | Biomarker-specific reagent + 2° Biomarker-specific reagent-Enzyme |
| with second signaling | conjugate + QM-Enzyme conjugate + Tyramide-Dye conjugate |
| conjugate comprising | Biomarker-specific reagent + 2° Biomarker-specific reagent-Enzyme |
| detectable moiety | conjugate + Tyramide-Enzyme conjugate + QM-Dye conjugate |
| | Biomarker-specific reagent + 2° Biomarker-specific reagent-Enzyme |
| | conjugate + Tyramide-Enzyme conjugate + Tyramide-Dye conjugate |
| G4. Signaling | Biomarker-specific reagent + 2° Biomarker-specific reagent-Enzyme |
| conjugate comprises | conjugate + Tyramide-(biotin/hapten) conjugate + Dye-(avidin/anti-hapten |
| member of a specific | Biomarker-specific reagent) conjugate |
| binding pair and other | Biomarker-specific reagent + 2° Biomarker-specific reagent-Enzyme |
| member of binding | conjugate + QM-(biotin/hapten) conjugate + Dye-(avidin/anti-hapten |
| pair is linked to | Biomarker-specific reagent) conjugate |
| detectable moiety | |
| G5. Signaling | Biomarker-specific reagent + 2° Biomarker-specific reagent-Enzyme |
| conjugate comprises | conjugate + QM-(biotin/hapten) conjugate + Enzyme-(avidin/anti-hapten |
| member of a specific | Biomarker-specific reagent) conjugate + DAB |
| binding pair and other | Biomarker-specific reagent + 2° Biomarker-specific reagent-Enzyme |
| member of binding | conjugate + QM-(biotin/hapten) conjugate + Enzyme-(avidin/anti-hapten |
| pair is linked to | Biomarker-specific reagent) conjugate + Chromogen |
| enzyme reactive with | Biomarker-specific reagent + 2° Biomarker-specific reagent-Enzyme |
| detectable moiety | conjugate + Tyramide-(biotin/hapten) conjugate + Enzyme-(avidin/anti- |
| | hapten Biomarker-specific reagent) conjugate + DAB |
| | Biomarker-specific reagent + 2° Biomarker-specific reagent-Enzyme |
| | conjugate + Tyramide-(biotin/hapten) conjugate + Enzyme-(avidin/anti- |
| | hapten Biomarker-specific reagent) conjugate + Chromogen |
| G6. Signaling | Biomarker-specific reagent + 2° Biomarker-specific reagent-Enzyme |
| conjugate comprises | conjugate + QM-(biotin/hapten) conjugate + Enzyme-(avidin/anti-hapten |
| member of a specific | Biomarker-specific reagent) conjugate + Tyramide-Dye conjugate |
| binding pair and other | Biomarker-specific reagent + 2° Biomarker-specific reagent-Enzyme |
| member of binding | conjugate + QM-(biotin/hapten) conjugate + Enzyme-(avidin/anti-hapten |
| pair is linked to | Biomarker-specific reagent) conjugate + QM-Dye conjugate |
| enzyme reactive with | Biomarker-specific reagent + 2° Biomarker-specific reagent-Enzyme |
| second detectable | conjugate + Tyramide-(biotin/hapten) conjugate + Enzyme-(avidin/anti- |
| moiety linked to a | hapten Biomarker-specific reagent) conjugate + Tyramide-Dye conjugate |
| detectable moiety | Biomarker-specific reagent + 2° Biomarker-specific reagent-Enzyme |
| | conjugate + Tyramide-(biotin/hapten) conjugate + Enzyme-(avidin/anti- |
| | hapten Biomarker-specific reagent) conjugate + QM-Dye conjugate |

H. Secondary detection reagent linked to member of specific binding pair

| | |
|---|---|
| H1. Dye linked to | Biomarker-specific reagent + 2° Biomarker-specific reagent-(biotin/hapten) |
| other member of | conjugate + Dye-(avidin/anti-hapten Biomarker-specific reagent) conjugate |
| specific binding pair | |
| H2. Enzyme linked | Biomarker-specific reagent + 2° Biomarker-specific reagent-(biotin/hapten) |
| to other member of | conjugate + Enzyme-(avidin/anti-hapten Biomarker-specific reagent) |
| specific binding pair, | conjugate + DAB |
| wherein the enzyme is | Biomarker-specific reagent + 2° Biomarker-specific reagent-(biotin/hapten) |
| reactive with | conjugate + Enzyme-(avidin/anti-hapten Biomarker-specific reagent) |
| detectable moiety | conjugate + Chromogen |
| | Biomarker-specific reagent + 2° Biomarker-specific reagent-(biotin/hapten) |
| | conjugate + Enzyme-(avidin/anti-hapten Biomarker-specific reagent) |
| | conjugate + QM-Dye conjugate |
| | Biomarker-specific reagent + 2° Biomarker-specific reagent-(biotin/hapten) |
| | conjugate + Enzyme-(avidin/anti-hapten Biomarker-specific reagent) |
| | conjugate + Tyramide-Dye conjugate |

I. Tertiary Biomarker-specific reagent linked directly to detectable moiety
Biomarker-specific reagent + 2° Biomarker-specific reagent + 3° Biomarker-specific reagent-Dye
conjugate
J. Tertiary Biomarker-specific reagent linked to Enzyme reacting with detectable moiety
Biomarker-specific reagent + 2° Biomarker-specific reagent + 3° Biomarker-specific reagent-
Enzyme conjugate + DAB
Biomarker-specific reagent + 2° Biomarker-specific reagent + 3° Biomarker-specific reagent-
Enzyme conjugate + Chromogen
K. Tertiary Biomarker-specific reagent linked to Enzyme reacting with detectable moiety

| | |
|---|---|
| K1. Signaling | Biomarker-specific reagent + 2° Biomarker-specific reagent + 3° |
| conjugate comprises | Biomarker-specific reagent-Enzyme conjugate + QM-Dye conjugate |
| detectable moiety | Biomarker-specific reagent + 2° Biomarker-specific reagent + 3° |
| | Biomarker-specific reagent-Enzyme conjugate + Tyramide-Dye conjugate |
| K2. Signaling | Biomarker-specific reagent + 2° Biomarker-specific reagent + 3° |
| conjugate comprises | Biomarker-specific reagent-Enzyme conjugate + QM-Enzyme conjugate + |
| enzyme that reacts | DAB |
| directly with | Biomarker-specific reagent + 2° Biomarker-specific reagent + 3° |
| detectable moiety | Biomarker-specific reagent-Enzyme conjugate + QM-Enzyme conjugate + |
| | Chromogen |
| | Biomarker-specific reagent + 2° Biomarker-specific reagent + 3° |
| | Biomarker-specific reagent-Enzyme conjugate + Tyramide-Enzyme |
| | conjugate + DAB |
| | Biomarker-specific reagent + 2° Biomarker-specific reagent + 3° |
| | Biomarker-specific reagent-Enzyme conjugate + Tyramide-Enzyme |
| | conjugate + Chromogen |
| K3. Signaling | Biomarker-specific reagent + 2° Biomarker-specific reagent + 3° |
| conjugate comprises | Biomarker-specific reagent-Enzyme conjugate + QM-Enzyme conjugate + |
| enzyme that reacts | QM-Dye conjugate |
| with second signaling | Biomarker-specific reagent + 2° Biomarker-specific reagent + 3° |
| conjugate comprising | Biomarker-specific reagent-Enzyme conjugate + QM-Enzyme conjugate + |
| detectable moiety | Tyramide-Dye conjugate |
| | Biomarker-specific reagent + 2° Biomarker-specific reagent + 3° |
| | Biomarker-specific reagent-Enzyme conjugate + Tyramide-Enzyme |
| | conjugate + QM-Dye conjugate |
| | Biomarker-specific reagent + 2° Biomarker-specific reagent + 3° |
| | Biomarker-specific reagent-Enzyme conjugate + Tyramide-Enzyme |
| | conjugate + Tyramide-Dye conjugate |
| K4. Signaling | Biomarker-specific reagent + 2° Biomarker-specific reagent + 3° |
| conjugate comprises | Biomarker-specific reagent-Enzyme conjugate + Tyramide-(biotin/hapten) |
| member of a specific | conjugate + Dye-(avidin/anti-hapten Biomarker-specific reagent) conjugate |
| binding pair and other | Biomarker-specific reagent + 2° Biomarker-specific reagent + 3° |
| member of binding | Biomarker-specific reagent-Enzyme conjugate + QM-(biotin/hapten) |
| pair is linked to | conjugate + Dye-(avidin/anti-hapten Biomarker-specific reagent) conjugate |
| detectable moiety | |
| K5. Signaling | Biomarker-specific reagent + 2° Biomarker-specific reagent + 3° |
| conjugate comprises | Biomarker-specific reagent-Enzyme conjugate + QM-(biotin/hapten) |
| member of a specific | conjugate + Enzyme-(avidin/anti-hapten Biomarker-specific reagent) |
| binding pair and other | conjugate + DAB |
| member of binding | Biomarker-specific reagent + 2° Biomarker-specific reagent + 3° |
| pair is linked to | Biomarker-specific reagent-Enzyme conjugate + QM-(biotin/hapten) |
| enzyme reactive with | conjugate + Enzyme-(avidin/anti-hapten Biomarker-specific reagent) |
| detectable moiety | conjugate + Chromogen |
| | Biomarker-specific reagent + 2° Biomarker-specific reagent + 3° |
| | Biomarker-specific reagent-Enzyme conjugate + Tyramide-(biotin/hapten) |
| | conjugate + Enzyme-(avidin/anti-hapten Biomarker-specific reagent) |
| | conjugate + DAB |
| | Biomarker-specific reagent + 2° Biomarker-specific reagent + 3° |
| | Biomarker-specific reagent-Enzyme conjugate + Tyramide-(biotin/hapten) |
| | conjugate + Enzyme-(avidin/anti-hapten Biomarker-specific reagent) |
| | conjugate + Chromogen |
| K6. Signaling | Biomarker-specific reagent + 2° Biomarker-specific reagent + 3° |
| conjugate comprises | Biomarker-specific reagent-Enzyme conjugate + QM-(biotin/hapten) |
| member of a specific | conjugate + Enzyme-(avidin/anti-hapten Biomarker-specific reagent) |
| binding pair and other | conjugate + Tyramide-Dye conjugate |
| member of binding | Biomarker-specific reagent + 2° Biomarker-specific reagent + 3° |
| pair is linked to | Biomarker-specific reagent-Enzyme conjugate + QM-(biotin/hapten) |
| enzyme reactive with | conjugate + Enzyme-(avidin/anti-hapten Biomarker-specific reagent) |
| second detectable | conjugate + QM-Dye conjugate |
| moiety linked to a | Biomarker-specific reagent + 2° Biomarker-specific reagent + 3° |
| detectable moiety | Biomarker-specific reagent-Enzyme conjugate + Tyramide-(biotin/hapten) |
| | conjugate + Enzyme-(avidin/anti-hapten Biomarker-specific reagent) |
| | conjugate + Tyramide-Dye conjugate |
| | Biomarker-specific reagent + 2° Biomarker-specific reagent + 3° |
| | Biomarker-specific reagent-Enzyme conjugate + Tyramide-(biotin/hapten) |
| | conjugate + Enzyme-(avidin/anti-hapten Biomarker-specific reagent) |
| | conjugate + QM-Dye conjugate |

L. Tertiary Biomarker-specific reagent linked to member of specific binding pair

| | |
|---|---|
| L1. Dye linked to | Biomarker-specific reagent + 2° Biomarker-specific reagent + 3° |
| other member of | Biomarker-specific reagent-(biotin/hapten) conjugate + Dye-(avidin/anti- |
| specific binding pair | hapten Biomarker-specific reagent) conjugate |
| L2. Enzyme linked | Biomarker-specific reagent + 2° Biomarker-specific reagent + 3° |
| to other member of | Biomarker-specific reagent-(biotin/hapten) conjugate + Enzyme- |
| specific binding pair, | (avidin/anti-hapten Biomarker-specific reagent) conjugate + DAB |
| wherein the enzyme is | Biomarker-specific reagent + 2° Biomarker-specific reagent + 3° |
| reactive with | Biomarker-specific reagent-(biotin/hapten) conjugate + Enzyme- |
| detectable moiety | (avidin/anti-hapten Biomarker-specific reagent) conjugate + Chromogen |
| | Biomarker-specific reagent + 2° Biomarker-specific reagent + 3° |
| | Biomarker-specific reagent-(biotin/hapten) conjugate + Enzyme- |
| | (avidin/anti-hapten Biomarker-specific reagent) conjugate + QM-Dye |
| | conjugate |
| | Biomarker-specific reagent + 2° Biomarker-specific reagent + 3° |
| | Biomarker-specific reagent-(biotin/hapten) conjugate + Enzyme- |
| | (avidin/anti-hapten Biomarker-specific reagent) conjugate + Tyramide-Dye |
| | conjugate |

Non-limiting examples of commercially available detection reagents or kits comprising detection reagents include: VENTANA ultraView detection systems (secondary antibodies conjugated to enzymes, including HRP and AP); VENTANA iVIEW detection systems (biotinylated anti-species secondary antibodies and streptavidin-conjugated enzymes); VENTANA OptiView detection systems (OptiView) (anti-species secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OptiView Amplification system (Anti-species secondary antibody conjugated to a hapten, an anti-hapten tertiary antibody conjugated to an enzyme multimer, and a tyramide conjugated to the same hapten); VENTANA DISCOVERY (e.g. DISCOVERY Yellow Kit, DISCOVERY Purple Kit, DISCOVERY Silver kit, DISCOVERY Red Kit, DISCOVERY Rhodamine Kit, etc.) DISCOVERY OmniMap, DISCOVERY UltraMap anti-hapten antibody, secondary antibody, chromogen, fluorophore, and dye kits, each of which are available from Ventana Medical Systems, Inc. (Tucson, Ariz.); PowerVision and PowerVision+IHC Detection Systems (secondary antibodies directly polymerized with HRP or AP into compact polymers bearing a high ratio of enzymes to antibodies); and DAKO EnVision™+ System (enzyme labeled polymer that is conjugated to secondary antibodies).

C4. Automated Systems

In an embodiment, the histochemical staining methods described herein are performed on an automated IHC staining device. Specific examples of automated IHC staining devices include: intelliPATH (Biocare Medical), WAVE (Celerus Diagnostics), DAKO OMNIS and DAKO AUTOSTAINER LINK 48 (Agilent Technologies), BENCHMARK XT (Ventana Medical Systems, Inc.), BENCHMARK Special Stains (Ventana Medical Systems, Inc.), BENCHMARK ULTRA (Ventana Medical Systems, Inc.), BENCHMARK GX (Ventana Medical Systems, Inc.), DISCOVERY XT (Ventana Medical Systems, Inc.), DISCOVERY ULTRA (Ventana Medical Systems, Inc.), Leica BOND, and Lab Vision Autostainer (Thermo Scientific). Automated IHC staining device are also described by Prichard, Overview of Automated Immunohistochemistry, Arch Pathol Lab Med., Vol. 138, pp. 1578-1582 (2014), incorporated herein by reference in its entirety. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published Patent Application Nos. 20030211630 and 20040052685, each of which is incorporated herein by reference in its entirety. The methods of the present invention may be adapted to be performed on any appropriate automated IHC staining device.

Automated IHC staining device typically implement staining protocols via a stainer unit that dispenses reagent onto a slide containing the sample to be stained. Commercially-available staining units typically operate on one of the following principles: (1) open individual slide staining, in which slides are positioned horizontally and reagents are dispensed as a puddle on the surface of the slide containing a tissue sample (such as implemented on the DAKO AUTOSTAINER Link 48 (Agilent Technologies) and intelliPATH (Biocare Medical) stainers); (2) liquid overlay technology, in which reagents are either covered with or dispensed through an inert fluid layer deposited over the sample (such as implemented on VENTANA BenchMark and DISCOVERY stainers); (3) capillary gap staining, in which the slide surface is placed in proximity parallel to another surface (which may be another slide or a coverplate) to create a narrow gap, through which capillary forces draw up and keep liquid reagents in contact with the samples (such as the staining principles used by DAKO TECHMATE, Leica BOND, and DAKO OMNIS stainers). Some iterations of capillary gap staining do not mix the fluids in the gap (such as on the DAKO TECHMATE and the Leica BOND). In some variations of capillary gap staining, the reagents are mixed in the gap, such as translating gap technology, in which a gap is created between the slide and a curved surface and movement of the surfaces relative to one another effects mixing (see U.S. Pat. No. 7,820,381); and dynamic gap staining, which uses capillary forces similar to capillary gap staining to apply sample to the slide, and then translates the parallel surfaces relative to one another to agitate the reagents during incubation to effect reagent mixing (such as the staining principles implemented on DAKO OMNIS slide stainers (Agilent)). It has recently been proposed to use inkjet technology to deposit reagents on slides. See WO 2016-170008 A1. This list of staining principles is not intended to be exhaustive, and the present methods and systems are intended to include any staining technology (both known and to be developed in the future) that can be used to apply the appropriate reagents to the sample.

The present invention is not limited to the use of automated systems. In some embodiments, the histochemical labeling methods described herein are applied manually. Or, particular steps may be performed manually while other steps are performed in an automated system.

C5. Counterstaining and Morphological Staining

If desired, the biomarker-stained slides may be counterstained to assist in identifying morphologically relevant areas and/or for identifying regions of interest (ROIs). Examples of counterstains include chromogenic nuclear counterstains, such as hematoxylin (stains from blue to violet), Methylene blue (stains blue), toluidine blue (stains nuclei deep blue and polysaccharides pink to red), nuclear fast red (also called Kernechtrot dye, stains red), and methyl green (stains green); non-nuclear chromogenic stains, such as eosin (stains pink); fluorescent nuclear stains, including 4′, 6-diamino-2-pheylindole (DAPI, stains blue), propidium iodide (stains red), Hoechst stain (stains blue), nuclear green DCS1 (stains green), nuclear yellow (Hoechst 5769121, stains yellow under neutral pH and stains blue under acidic pH), DRAQ5 (stains red), DRAQ7 (stains red); fluorescent non-nuclear stains, such as fluorophore-labeled phalloidin, (stains filamentous actin, color depends on conjugated fluorophore).

In certain embodiments, a serial section of the biomarker-stained section (or the biomarker-stained section itself) may be morphologically stained. Basic morphological staining techniques often rely on staining nuclear structures with a first dye, and staining cytoplasmic structures with a second stain. Many morphological stains are known, including but not limited to, hematoxylin and eosin (H&E) stain and Lee's Stain (Methylene Blue and Basic Fuchsin). Examples of commercially available H&E stainers include the VENTANA SYMPHONY (individual slide stainer) and VENTANA HE 600 (individual slide stainer) H&E stainers from Roche; the Dako CoverStainer (batch stainer) from Agilent Technologies; the Leica ST4020 Small Linear Stainer (batch stainer), Leica ST5020 Multistainer (batch stainer), and the Leica ST5010 Autostainer XL series (batch stainer) H&E stainers from Leica Biosystems Nussloch GmbH.

D. Multiplex Staining Method

As noted above, in an embodiment, the colorectal samples are stained by a multiplex method. Multiplex methods involve differential staining of different biomarkers in a single tissue section.

One way to accomplish differential staining of different biomarkers is to select combinations of biomarker-specific reagents and detection reagents that will not result in off-target cross-reactivity between different antibodies or detection reagents (termed “combination staining”). In such an example, all biomarker-specific reagents are bound to the sample before any of the detection reagents are applied. In these examples, the biomarker-specific reagents and the detection reagents must be selected such that a first set of detection reagents will react only with a first biomarker specific reagent and a second set of detection reagents will react only with a second biomarker-specific reagent, regardless of whether both biomarker-specific reagents are present. Thus, for example, where the biomarker specific reagents are antibodies, the EGFR antibody may be selected from a first species (such as a mouse anti-human EGFR monoclonal antibody, rat anti-human EGFR monoclonal antibody, or rabbit anti-human EGFR monoclonal antibody), the EREG antibody may be selected from a second species (such as a mouse anti-human EREG monoclonal antibody, rat anti-human EREG monoclonal antibody, or rabbit anti-human EREG monoclonal antibody, with the proviso that the second species is different from the first species of antibody), and the AREG antibody may be selected from a third species (such as a mouse anti-human AREG monoclonal antibody, rat anti-human AREG monoclonal antibody, or rabbit anti-human AREG monoclonal antibody, with the proviso that the third species is different from the first and second species). In such an embodiment, secondary antibodies may be provided having different species specificities to allow for differential staining of the different targets. In another embodiment, tagged biomarker-specific reagents may be used (for example, bearing hapten tags, epitope tags, etc.). In such a case, different tags on the different biomarker-specific reagents facilitate binding of different sets of detection reagents to the sample. Thus, for example, where the biomarker specific reagents are antibodies, they could be coupled to different hapten or epitope tags, and the secondary antibodies are selected to specifically bind to the hapten or epitope tag. Additionally, each set of detection reagents should be adapted to deposit a different detectable entity on the section, such as by depositing a different enzyme in proximity to each specific binding agent. Such arrangements have the potential advantage of being able to have each set of biomarker-specific reagents and associated detection reagents present on the sample at the same time and/or to perform staining with cocktails of biomarker-specific reagents and/or detection reagents, thereby reducing the number of staining steps. However, such arrangements may not always be feasible, as reagents may cross-react with different enzymes, and the various antibodies may cross-react with one another, leading to aberrant staining.

Another way to accomplish differential labeling of different biomarkers is to sequentially stain the sample for each biomarker. In such an embodiment, a first biomarker-specific reagent is reacted with the section, followed by a secondary detection reagent to the first biomarker-specific reagent and other detection reagents resulting in deposition of a first detectable moiety. The section is then treated to remove the biomarker-specific reagent and associated detection reagents from the section while leaving the deposited stain in place. The process is repeated for subsequent biomarker-specific reagent. Examples of methods for removing the biomarker-specific reagent and associated detection reagents include heating the sample in the presence of a buffer that elutes the antibodies from the sample (termed a “heat-kill method”), such as those disclosed by Stack et al., Multiplexed immunohistochemistry, imaging, and quantitation: A review, with an assessment of Tyramide signal amplification, multispectral imaging and multiplex analysis, Methods, Vol. 70, Issue 1, pp. 46-58 (November 2014), and PCT/EP2016/057955, the contents of which are incorporated by reference.

As will be appreciated by the skilled artisan, combination staining and sequential staining methods may be combined. For example, where only a subset of the biomarker-specific reagents are compatible with combination staining, the sequential staining method can be modified, wherein the biomarker-specific reagents compatible with combination staining are applied to the sample using a combination staining method, and the remaining antibodies are applied using a sequential staining method.

In an embodiment, a multiplex method is provided comprising contacting a single tissue section of an FFPE colorectal tumor sample with:
a human EGFR protein biomarker-specific reagent and detection reagents sufficient to deposit a first chromogen in proximity to the EGFR protein biomarker-specific reagent bound to the tissue section; and
a human AREG protein biomarker-specific reagent and detection reagents sufficient to deposit a second chromogen in proximity to the human AREG protein biomarker-specific reagent bound to the tissue section.
   In an embodiment, a multiplex method is provided comprising contacting a single tissue section of an FFPE colorectal tumor sample with:
a human EGFR protein biomarker-specific reagent and detection reagents sufficient to deposit a first chromogen in proximity to the EGFR protein biomarker-specific reagent bound to the tissue section; and
a human EREG protein biomarker-specific reagent and detection reagents sufficient to deposit a second chromogen in proximity to the EREG protein biomarker-specific reagent bound to the tissue section.
   In an embodiment, a multiplex method is provided comprising contacting a single tissue section of an FFPE colorectal tumor sample with:
a human EGFR protein biomarker-specific reagent and detection reagents sufficient to deposit a first chromogen in proximity to the EGFR protein biomarker-specific reagent bound to the tissue section; and
a human EREG protein biomarker-specific reagent, a human AREG protein biomarker-specific reagent and detection reagents sufficient to deposit a second chromogen in proximity to the human EREG protein biomarker-specific reagent and the a human AREG protein biomarker-specific reagent bound to the tissue section.
   In an embodiment, a multiplex method is provided comprising contacting a single tissue section of an FFPE colorectal tumor sample with:
a human EGFR protein biomarker-specific reagent and detection reagents sufficient to deposit a first chromogen in proximity to the EGFR protein biomarker-specific reagent bound to the tissue section; and
a human EREG protein biomarker-specific reagent and detection reagents sufficient to deposit a second chromogen in proximity to the human EREG protein biomarker-specific reagent bound to the tissue section; and
a human AREG protein biomarker-specific reagent and detection reagents sufficient to deposit a third chromogen in proximity to the human AREG protein biomarker-specific reagent bound to the tissue section.
   In these exemplary embodiments, the biomarkers may be labeled in a particular order as desired. For example, EGFR may be labeled first prior to the one or more EGFR ligands. Or, one or both EGFR ligands may be labeled before EGFR is labeled. Or, one EGFR ligand may be labeled before EGFR, and one EGFR ligand may be labeled after EGFR is labeled. The ease of detection of the detectable moieties (e.g., chromogens) may influence the order in which they are used. For example, the detectable moiety (e.g., chromogen) that is easiest to detect may be selected for the biomarker that is the least prevalent. Likewise, the detectable moiety (e.g., chromogen) that is the hardest to detect may be selected for the biomarker that is the most prevalent.

The detectable moiety (e.g., chromogen) used to detect EGFR may be different from the detectable moiety (e.g., chromogen) used to detect AREG and/or the detectable moiety (e.g., chromogen) used to detect EREG. In some embodiments, the detectable moiety (e.g., chromogen) used to detect EGFR is the same as the detectable moiety (e.g., chromogen) used to detect AREG. In some embodiments, the detectable moiety (e.g., chromogen) used to detect EGFR is the same as the detectable moiety (e.g., chromogen) used to detect EREG. In some cases, the extent of the ligand expression (regardless of identity) may be predictive. In some embodiments, the detectable moiety (e.g., chromogen) used to detect AREG is the same as the detectable moiety (e.g., chromogen) used to detect EREG.

### III. IMAGE PROCESSING AND ANALYSIS

In an embodiment, digital images of the stained tissue sections obtained according to the above methods may be obtained. Following staining of the tissue section(s), the samples may undergo image acquisition, as well as image processing and analysis. The digital images may be useful, for example, for long-term archiving of test results and/or for digital analysis of staining patterns. In another embodiment, the digital images may be used in a digital analysis of a cohort of tumors from patients with known outcomes to develop a scoring algorithm for evaluating EGFR and EGFR ligand expression. In another embodiment, the digital images may be fed into a diagnostic analysis system trained to aid in the evaluation for stained samples for prediction of response to EGFR-directed therapies.

A. Image Acquisition

The tissue section(s) are transported to an imaging apparatus or image acquisition system for obtaining digital images of the tissue section(s). The image acquisition system may comprise a scanning platform such as a slide scanner that can scan the stained slides at 20×, 40×, or other magnifications to produce high resolution whole-slide digital images, including for example slide scanners. At a basic level, the typical slide scanner includes at least: (1) a microscope with lens objectives, (2) a light source (such as halogen, light emitting diode, white light, and/or multispectral light sources, depending on the dye), (3) robotics to move glass slides around (or to move the optics around the slide), (4) one or more digital cameras for image capture, (5) a computer and associated software to control the robotics and to manipulate, manage, and view digital slides. Digital data at a number of different X-Y locations (and in some cases, at multiple Z planes) on the slide are captured by the camera's charge-coupled device (CCD), and the images are joined together to form a composite image of the entire scanned surface. Common methods to accomplish this include: (1) Tile based scanning, in which the slide stage or the optics are moved in very small increments to capture square image frames, which overlap adjacent squares to a slight degree. The captured squares are then automatically matched to one another to build the composite image; and (2) Line-based scanning, in which the slide stage moves in a single axis during acquisition to capture a number of composite image “strips.” The image strips can then be matched with one another to form the larger composite image.

A detailed overview of various scanners (both fluorescent and brightfield) can be found at Farahani et al., Whole slide imaging in pathology: advantages, limitations, and emerging perspectives, Pathology and Laboratory Medicine Intl, Vol. 7, p. 23-33 (June 2015), the content of which is incorporated by reference in its entirety. Examples of commercially available slide scanners include: 3DHistech PANNORAMIC SCAN II; DigiPath PATHSCOPE; Hamamatsu NANOZOOMER RS, HT, and XR; Huron TISSUESCOPE 4000, 4000XT, and HS; Leica SCANSCOPE AT, AT2, CS, FL, and SCN400; Mikroscan D2; Olympus VS120-SL; Omnyx VL4, and VL120; PerkinElmer LAMINA; Philips ULTRA-FAST SCANNER; Sakura Finetek VISIONTEK; Unic PRECICE 500, and PRECICE 600x; VENTANA ISCAN COREO and ISCAN HT; and Zeiss AXIO SCAN.Z1. Other exemplary systems and features can be found in, for example, WO2011-049608) or in U.S. Patent Application No. 61/533,114, filed on Sep. 9, 2011, entitled IMAGING SYSTEMS, CASSETTES, AND METHODS OF USING THE SAME the content of which is incorporated by reference in its entirety.

Images generated by the scanning platform may be transferred to an image analysis system, to a server or database accessible by an image analysis system, or to a non-transitory digital storage medium. In some embodiments, the images may be transferred automatically via one or more local-area networks and/or wide-area networks. In some embodiments, the image analysis system may be integrated with or included in the scanning platform and/or other modules of the image acquisition system, in which case the image may be transferred to the image analysis system. In some embodiments, the image acquisition system may not be communicatively coupled to the image analysis system, in which case the images may be stored on a non-volatile storage medium of any type (e.g., a flash drive) and downloaded from the medium to the image analysis system or to a server or database communicatively coupled thereto.

B. Image Analysis

In an embodiment, the digital image is analyzed by an image analysis system. In such an embodiment, the image(s) acquired as described above are processed by an image analysis system, including at least a processor and a memory coupled to the processor, the memory to store computer-executable instructions that, when executed by the processor, cause the processor to perform operations.

The image analysis system may feature one or more computing devices such as desktop computers, laptop computers, tablets, smartphones, servers, application-specific computing devices, or any other type(s) of electronic device(s) capable of performing the techniques and operations described herein. In some embodiments, the image analysis system may be implemented as a single device. In other embodiments, the image analysis system may be implemented as a combination of two or more devices together. For example, an image analysis system may include one or more server computers and a one or more client computers communicatively coupled to each other via one or more local-area networks and/or wide-area networks such as the Internet.

The image analysis system may include a memory, a processor, and a display. The memory may include any combination of any type of volatile or non-volatile memories, such as random-access memories (RAMs), read-only memories such as an Electrically-Erasable Programmable Read-Only Memory (EEPROM), flash memories, hard drives, solid state drives, optical discs, and the like. The processor may include one or more processors of any type, such as central processing units (CPUs), graphics processing units (GPUs), special-purpose signal or image processors, field-programmable gate arrays (FPGAs), tensor processing units (TPUs), and so forth.

The display may be implemented using any suitable technology, such as LCD, LED, OLED, TFT, Plasma, etc. In some implementations, display may be a touch-sensitive display (a touchscreen).

Images generated by the scanning platform may be transferred to an image analysis system or to a server or database accessible by the image analysis system. In some embodiments, the images may be transferred automatically via one or more local-area networks and/or wide-area networks. In some embodiments, the image analysis system may be integrated with or included in the scanning platform and/or other modules of the image acquisition system, in which case the image may be transferred to the image analysis system. In some embodiments, the image acquisition system may not be communicatively coupled to the image analysis system, in which case the images may be stored on a non-volatile storage medium of any type (e.g., a flash drive, a hard drive, etc.) and downloaded from the medium to the image analysis system or to a server or database communicatively coupled thereto.

The skilled artisan will appreciate that the biological image analysis device described herein may be included within systems comprising additional components, e.g. analyzers, scanners, etc. For example, the biological image analyzer may be communicatively coupled to a computer-readable storage medium containing a digital copy of the image of the biological sample. Alternatively, the biological image analysis device may be communicatively coupled to an imaging apparatus.

The skilled artisan will also appreciate that additional modules or databases may be incorporated into the workflow. For example, an image processing module may be run to apply certain filters to the acquired images or to identify certain histological and/or morphological structures within the tissue samples. In addition, a region of interest (ROI) selection module may be utilized to select a particular portion of an image for analysis. Likewise, an unmixing module may be run to provide image channel images corresponding to a particular stain or biomarker.

The image analysis system may also include an object identifier, an ROI generator, a user-interface module, and/or a scoring engine. It can be appreciated by persons having ordinary skill in the art that each module may be implemented as a number of sub-modules, and that any two or more modules can be combined into a single module. Furthermore, in some embodiments, the system may include additional engines and modules (e.g., input devices, networking and communication modules, etc.). Exemplary commercially-available software packages useful in implementing modules as disclosed herein include VENTANA VIRTUOSO software suite (Ventana Medical Systems, Inc.); TISSUE STUDIO, DEVELOPER XD, and IMAGE MINER software suites (Definiens); BIOTOPIX, ONCOTOPIX, and STEREOTOPIX software suites (Visiopharm); and the HALO platform (Indica Labs, Inc.).

For biomarkers that are scored on the basis of the biomarker's association with a particular type of object (such as membranes, etc.), the features extracted by the object identifier may include features or feature vectors sufficient to categorize the objects in the sample as biomarker-positive objects of interest or biomarker-negative markers of interest and/or by level or intensity of biomarker staining of the object. In cases where the biomarker may be weighted differently depending on the object type that is expressing it, the features extracted by the object identifier may include features relevant to determining the type of objects associated with biomarker-positive pixels. Thus, the objects may then be categorized at least on the basis of biomarker expression (for example, biomarker-positive or biomarker-negative cells) and, if relevant, a sub-type of the object (e.g. tumor cell, etc.). In cases where extent of biomarker-expression is scored regardless of association with objects, the features extracted by the object identifier may include, for example, location and/or intensity of biomarker-positive pixels. The precise features extracted from the image will depend on the type of classification function being applied, and would be well known to a person of ordinary skill in the art.

The image analysis system may also pass the image to an ROI generator. The ROI generator may be used to identify the ROI or ROIs of the image from which the score will be calculated. There may be cases where the object identifier is not applied to the whole image and the ROI or ROIs generated by the ROI generator are used to define a subset of the image on which the object identifier is executed.

The object identifier and the ROI generator may be implemented in any order. For example, the object identifier may be applied to the entire image first. The positions and features of the identified objects may then be stored and recalled when the ROI generator is implemented. Alternatively, the ROI generator can be implemented first. In this case, the object identifier may be implemented only on the ROI, or it may still be implemented on the whole image. It may also be possible to implement the object identifier and the ROI generator simultaneously.

In an embodiment, the memory of the image analysis system instructs the processor to perform a set of functions comprising: (a) unmixing a digital image of a stained slide as described herein to obtain a deconvoluted image for each chromogen used to stain the slide (and optionally a counterstain used to stain the slide); and (b) identifying one or more object(s) of interest in the deconvoluted image and extracting one or more object metric(s) from the object(s) of interest. In an embodiment, the set of objects and associated object metrics may be used, for example, for developing a predictive scoring algorithm for identifying patients responsive to an EGFR-directed therapy. In another embodiment, the image analysis system may further comprise a scoring engine, wherein the scoring engine applies a predictive scoring function to a feature vector comprising a set of object metrics for human EGFR protein for a colorectal tumor of a subject and a set of object metrics for either or both of human AREG protein and human EREG protein for a colorectal tumor of a subject, wherein the output of the predictive scoring function is a score indicative of whether the colorectal tumor is likely to respond to an EGFR-directed therapy. In another embodiment, the memory of the image analysis system instructs the processor to perform a set of functions comprising executing a scoring guide on the image, wherein the scoring guide comprises a plurality of classifiable subsets, wherein the classifiable subsets based application of a clustering function to a plurality of extracted features of a plurality of objects of interest.

B1. Unmixing

Unmixing is a procedure by which the measured spectrum of a mixed pixel is decomposed into a collection of constituent spectra and a set of corresponding fractions that indicate the proportion of each constituent spectrum present in the pixel. Specifically, the unmixing process can extract stain-specific channels to determine local concentrations of individual stains using reference spectra that are well known for standard types of tissue and stain combinations. The unmixing may use reference spectra retrieved from a control image or estimated from the image under observation. Unmixing the component signals of each input pixel enables retrieval and analysis of stain-specific channels, such as hematoxylin channel and eosin channel in H&E images, or a diaminobenzidine (DAB) channel and a counterstain (e.g., hematoxylin) channel in IHC images. The terms “unmixing” and “color deconvolution” (or “deconvolution”) or the like (e.g. “deconvolving,” “unmixed”) are used interchangeably in the art. Several techniques have been proposed to decompose each pixel of the RGB image into a collection of constituent stains and the fractions of the contributions from each of them, including (but not limited to) processes described by Ruifrok et al., (Anal. Quant. Cytol. Histol., 2001, 23:291-299), Chen and Srinivas (Comput Med Imaging Graph, 2015, 46(1):30-39), Kesheva (Lincoln Laboratory Journal, 2003, 14:55-78), Greer (IEEE Trans Image Proc., 2012, 221:219-228), and Yang et al. (IEEE Trans. Image Proc., 2011, 20:1112-1125).

In an embodiment, the digital image(s) obtained as described above are deconvoluted into separate deconvoluted image(s) for each chromogen. Thus, for example, a multiplex stained slide may be provided, the slide stained with a first chromogen for EGFR and at least a second chromogen for one or more of EREG and AREG, and a digital image of the stained slide may be deconvoluted on the basis of a channel for each of the chromogens.

B2. Object Identification

In an embodiment, objects are identified in the deconvoluted images. The “objects” are structures or staining patterns within the tumor sample that are used to evaluate and quantitate biomarker staining. Examples include biomarker-positive cells (e.g., EGFR positive cells, AREG positive cells, EREG positive cells, and/or EGFR ligand-positive cells); biomarker-positive membrane (e.g., EGFR positive membrane, AREG positive membrane, EREG positive membrane, and/or EGFR ligand-positive membrane); biomarker-positive punctate membrane staining patterns (e.g., EGFR positive punctate staining, AREG positive punctate staining, EREG positive punctate staining, and/or EGFR ligand-positive punctate staining); biomarker-positive cytoplasm (e.g., EGFR positive cytoplasm, AREG positive cytoplasm, EREG positive cytoplasm, and/or EGFR ligand-positive cytoplasm); biomarker-positive cell clusters (e.g., regions exceeding a predefined area having a density of biomarker-positive cells that exceeds a predefined threshold, e.g., EGFR positive cell clusters, AREG positive cell clusters, EREG positive cell clusters, and/or EGFR ligand-positive cell clusters); biomarker-positive tumor cells (e.g., EGFR positive tumor cells, AREG positive tumor cells, EREG positive tumor cells, and/or EGFR ligand-positive tumor cells); biomarker-positive membrane associated with tumor cells (e.g., EGFR positive membrane associated with tumor cells, AREG positive membrane associated with tumor cells, EREG positive membrane associated with tumor cells, and/or EGFR ligand-positive membrane associated with tumor cells); biomarker-positive cytoplasm associated with tumor cells (e.g., EGFR positive cytoplasm associated with tumor cells, AREG positive cytoplasm associated with tumor cells, EREG positive cytoplasm associated with tumor cells, and/or EGFR ligand-positive cytoplasm associated with tumor cells); etc.

In an embodiment, the image analysis system executes an object identifier function on one or more of the deconvuluted images to identify and mark relevant objects and other features within the image that may later be used for scoring. The object identifier may extract from (or generate for) each image a plurality of image features characterizing the various objects in the image as a well as pixels representing expression of the biomarker(s). The values of the plurality of image features may be combined into a high-dimensional vector, hereinafter referred to as the “feature vector” characterizing the expression of the biomarker.

For biomarkers that are scored on the basis of the biomarker's association with a particular type of object (such as membranes, etc.), the features extracted by the object identifier may include features or feature vectors sufficient to categorize the objects in the sample as biomarker-positive objects of interest or biomarker-negative objects of interest and/or by level or intensity of biomarker staining of the object. In cases where the biomarker may be weighted differently depending on the object type that is expressing it, the features extracted by the object identifier may include features relevant to determining the type of objects associated with biomarker-positive pixels. Thus, the objects may then be categorized at least on the basis of biomarker expression (for example, biomarker-positive or biomarker-negative cells) and, if relevant, a sub-type of the object (e.g. tumor cell, etc.). In cases where extent of biomarker-expression is scored regardless of association with objects, the features extracted by the object identifier may include, for example, location and/or intensity of biomarker-positive pixels. The precise features extracted from the image will depend on the type of classification function being applied, and would be well known to a person of ordinary skill in the art.

In some embodiments, it may be desirable to limit image analysis to certain regions of interest (ROIs) that define biologically significant region(s) in which the biomarkers are detected and/or quantitated. General examples of morphological regions of a tumor-containing tissue section that may be considered a ROI include: a whole tumor (WT) region, an invasive margin (IM) region, a tumor core (TC) region; and a peri-tumoral (PT) region. In some embodiments, the ROI is identified in a whole slide image in order to detect all tissue regions in the ROI while limiting the amount of background non-tissue area that is analyzed. In some embodiments, the ROI is identified in a digital image of a first serial section of the test sample, being stained with a morphological stain (such as an H&E-stained image), and the ROI is automatically registered to a digital image of at least a second serial section of the test sample, being stained with another stain. In some embodiments, the ROI is identified in a digital image of a first serial section of the test sample, being stained with H&E, and the ROI is automatically registered to a digital image of at least a second serial section, a third serial section of the test sample, and a fourth serial section of the test sample.

The ROI may be limited to the morphological region, may be expanded to include regions outside of the morphological region (i.e. by extending the margin of the ROI a defined distance outside of the morphological region), or may be restricted to a sub-region of the morphological region (for example, by shrinking the ROI a defined distance inside of the circumference of the morphological region or by identifying regions within the ROI having certain characteristics (such as a baseline density of certain cell types)). Where the morphological region is an edge region, the ROI may be defined as, for example, all points within a defined distance of any point of the edge, all points on one side of the edge within a defined distance of any point of the edge, a minimal geometric region (such as a circle, oval, square, rectangle, etc.) encompassing the entire edge region, all points within a circle having a defined radius centered on a center point of the edge region, etc.

Related to the presently disclosed biomarkers, ROIs may also include biomarker-positive cell clusters or points within a defined distance of a biomarker-positive cell cluster (such as an EGFR-positive tumor region). In some embodiments, the same ROI may be used for all sections and biomarkers. For example, a morphologically defined ROI may be identified in an H&E-stained section of the sample and used for all biomarker-stained sections. In other embodiments, different ROIs may be used for different biomarkers (for example, EGFR may be identified in a whole tumor region, while EGFR ligand analysis is confined only to EGFR-rich regions).

In some embodiments, a ROI identification module may be used to select a portion of the biological sample for which an image or for which image data should be acquired, e.g. a region of interest having a large concentration of fibroblast cells. In some embodiments, the ROI is identified by a user of a system of the present disclosure, or another system communicatively coupled to a system of the present disclosure. Alternatively, and in other embodiments, the region selection module retrieves a location or identification of a region or interest from a storage/memory. In some embodiments, the ROI identification module automatically generates a ROI, for example, via methods described in PCT/EP2015/062015, the disclosure of which is hereby incorporated by reference herein in its entirety. In some embodiments, the ROI is automatically determined by the system based on some predetermined criteria or characteristics that are in or of the image (e.g. for a biological sample stained with more than two stains, identifying an area of the image that comprises just two stains). The region selection module then outputs the ROI. In certain embodiments, the ROI identification module generates a graphic user interface comprising the digital image, and a trained expert (such as a pathologist) manually delineates one or more morphological region(s) in the digital image as the ROI. In other embodiments, a computer-implemented system may assist the user in annotating the ROI (termed, “semi-automated ROI annotation”). For example, the user may delineate one or more regions on the digital image, which the system then automatically transforms into a complete ROI. For example, if the desired ROI is an WT region, a user can delineate (e.g., by outlining, tracing) a WT region, and the system applies a pattern recognition function that uses computer vision and machine learning to identify regions having similar morphological characteristics to an WT region. Many other arrangements could be used as well. In cases in which ROI generation is semi-automated, the user may be given an option to modify the ROI annotated by the computer system, such as by expanding the ROI, annotating regions of the ROI or objects within the ROI to be excluded from analysis, etc. In some embodiments, a pathologist annotates the tumor, and a software system is used for identifying object metrics. In some embodiments, an image is obtained (of a tumor), the image is scanned, the pathologist annotates the tumor/image, and then an output is generated. In other embodiments, the computer system may automatically suggest an ROI without any direct input from the user (termed an “automated ROI annotation”). For example, a previously-trained tissue segmentation function or other pattern recognition function may be applied to an unannotated image to identify the desired morphological region to use as an ROI. The user may be given an option to modify the ROI annotated by the computer system, such as by expanding the ROI, annotating regions of the ROI or objects within the ROI to be excluded from analysis, etc.

In an embodiment, the ROI is annotated directly in the digital image of the sample stained for the biomarkers, in which case the ROI is carried over into the deconvoluted image. In other embodiments, the ROI is annotated in a digital image of a serial section of the biomarker-stained sample, and the annotated ROI is registered to the digital image(s) of the biomarker-stained samples. In such an embodiment, the image analysis system may execute a registration function that transfers annotations onto adjacent slides, taking position, orientation, and local deformations of the tissue section into account. The registration function may further include functions that allow a user to edit the annotations, for example, by allowing shifting annotations, rotating annotations, locally modifying their outlines, delineating staining artifacts, etc. Exemplary registration functions are disclosed at, for example, US 2016/0321495 A1, the content of which is incorporated herein by reference. In an embodiment, a set of images generated from a simplex staining methodology is provided, wherein a serial section of each simplex stained sample is provided, the serial section being stained with a morphological stain (such as H&E), and wherein the ROI(s) is/are annotated on a digital image of the morphological stained sample and registered to the biomarker-stained serial section(s) (or deconvoluted images thereof). In another embodiment, a set of images generated from a multiplex staining methodology is provided, wherein a serial section of the multiplex-stained sample is provided, the serial section being stained with a morphological stain (such as H&E), and wherein the ROI(s) is/are annotated on a digital image of the morphological stained sample and registered to the biomarker-stained serial section (or deconvoluted images thereof).

In an embodiment, an object metric is calculated by applying a metric of the ROI to the raw object counts. Examples of ROI metrics that could be used for object metric calculation include: area of the ROI; total number of cells within the ROI; total number of specific cell types within the ROI (such as tumor cells, immune cells, stromal cells, cells positive for a first biomarker, etc.), length of an edge defining the ROI (such as circumference of the ROI, or a length of a centerline bisecting the ROI), number of cells defining the edge of the ROI, etc. Examples of object metrics related to select objects are set forth in Table 6:

TABLE 6

| Objects | Object Metrics |
|---|---|
| Marker positive cells and | Area density (# marker positive cells over area of ROI) |
| tumor cells | Tumor density (# marker positive cells over area of tumor cells in ROI) |
| | Total cell ratio (# of marker positive cells to number of a total cells in ROI) |
| | Tumor cell ratio (# of marker-positive cells to number of tumor cells in ROI) |
| | Tumor fraction (#/area of marker-positive tumor cells to number/area of total |
| | tumor cells in ROI) |
| | Marker-marker ratio (# of marker 1-positive cells to # of marker2-positive |
| | cells) |
| | Marker-marker tumor ratio (# of marker1-positive tumor cells to # of |
| | marker2-positive tumor cells) |
| | Marker2 cell to Marker2 tumor cells (# of marker1-positive cells to # of |
| | marker2-positive tumor cells) |
| Marker-positive | Area fraction/percentage (Area of marker-positive staining pattern over area of |
| membrane, punctate, or | ROI) |
| cytoplasm staining | Membrane fraction/percentage (Amount/area of marker-positive staining |
| patterns | pattern over amount/area of total membrane/cytoplasm) |
| | Membrane to Tumor membrane fraction/percentage (Amount/area of marker- |
| | positive staining pattern over amount/area of tumor cell membrane/cytoplasm) |
| | Tumor fraction/percentage (Amount/area of marker-positive staining pattern |
| | in tumor membrane/cytoplasm over amount/area of tumor cell |
| | membrane/cytoplasm) |
| | Marker-marker ratio (Area markerl-positive staining pattern to area of |
| | marker2-staining pattern) |
| | Area density (#cells with marker-positive staining pattern over area of ROI) |
| | Tumor density (#cells with marker-positive staining pattern over area of tumor |
| | cells in ROI) |
| | Total cell ratio (#cells with marker-positive staining pattern to number of a |
| | total cells in ROI) |
| | Tumor cell ratio (#cells with marker-positive staining pattern to number of |
| | tumor cells in ROI) |
| | Tumor fraction (#/area of tumor cells with marker-positive staining pattern to |
| | number/area of total tumor cells in ROI) |
| | Marker-marker ratio (# of cells with marker1-positive staining pattern to # of |
| | cells with marker2-positive staining pattern) |
| | Marker-marker tumor ratio (# of tumor cells with marker1-positive staining |
| | pattern to # of tumor cells with marker2-positive staining pattern) |
| | Marker1 cell to Marker2 tumor cells (# of cells with marker1-positive staining |
| | pattern to # of tumor cells with marker2-positive staining pattern) |
| | * “Marker1” and “Marker2: may each be EGFR, EREG, AREG, or EREG + |
| | AREG, for example. |
| Marker-positive cell | Area fraction/percentage (Area of marker-positive cell cluster(s) over area of |
| clusters (e.g. EGFR- | ROI) |
| positive cell clusters) | Tumor area ratio (Area marker-positive cell cluster(s) over area of tumor cells |
| | in ROI) |
| | Total cell ratio (# of cells in marker-positive cell cluster(s) to number of a total |
| | cells in ROI) |
| | Tumor cell ratio (# of cells in marker-positive cell cluster(s) to number of |
| | tumor cells in ROI) |
| | Tumor fraction (#/area of marker-positive tumor cells in marker-positive cell |
| | cluster(s) to number/area of total tumor cells in ROI) |
| | Marker-marker ratio (# of marker1-positive cells in markerl-positive cell |
| | cluster(s) to # of marker2-positive cells in marker1-positive cell cluster(s)) |
| | Marker-marker tumor ratio (# of marker1-positive tumor cells in marker1- |
| | positive cell cluster(s) to # of marker2-positive tumor cells in marker1- |
| | positive cell cluster(s)) |
| | Marker1 tumor cell to Marker2 cells (# of marker1-positive tumor cells in |
| | marker1-positive cell cluster(s) to # of marker2-positive tumor cells in |
| | marker1-positive cell cluster(s)) |
| | * “Marker1” and “Marker2” may each be EGFR, EREG, AREG, or EREG + |
| | AREG, for example. |

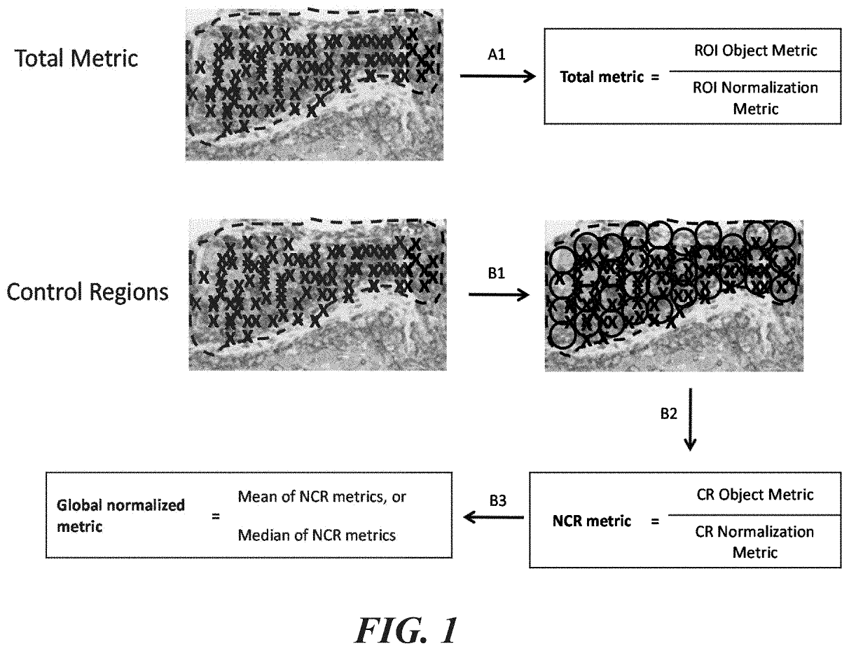

The object metric may be based directly on the raw counts in the ROI (referred to hereafter as a “Total metric”), or based on a mean or median object metric of a plurality of control regions within the ROI (hereafter referred to as a “global metric”). These two approaches are illustrated at FIG. 1. In both cases, an image of an slide is provided having an ROI annotated (denoted as the region within the dashed line) and objects of interest identified. For the total metric approach, the feature metric is calculated by quantitating the relevant metric of all the marked features within the ROI (“ROI object metric”) and dividing the ROI object metric (such as total marked objects or total area of marked biomarker expression, etc.) by the ROI metric (such as the area of the ROI, number of total cells within ROI, etc.) (step A1). For the global metric approach, a plurality of control regions (illustrated by the open circles) is overlaid on the ROI (step B1). A control region metric (“CR metric”) is calculated by quantitating the relevant metric of the control region (“CR Object Metric”) (such as total marked objects within the control region or total area of marked biomarker expression within the control region, etc.) and dividing it by a control region ROI metric (“CR ROI Metric”) (such as the area of the control region, number of total cells within the control region, etc.) (step B2). A separate CR metric is calculated for each control region. The global metric is obtained by calculating the mean or the median of all CR metrics (Step B3).

Where control regions are used, any method of overlaying control regions for metric processing may be used. In a specific embodiment, the ROI may be divided into a plurality of grid spaces (which may be equal sized, randomly sized, or some combination of varying sizes), each grid space constituting a control region. Alternatively, a plurality of control regions having known sizes (which may be the same or different) may be placed adjacent to each other or overlapping one another to cover substantially the entire ROI. Other methods and arrangements may also be used, so long as the output is an object metric for the ROI that can be compared across different samples. Specific examples of ROI, object, and object metric combinations useful in evaluating the images of the stained samples disclosed herein include (but are not necessarily limited to) those set forth below in Table 7. In each case, the “object metric(s)” in Table 7 may refer to a total metric, to a control region metric, or to a global metric.

TABLE 7

| ROI | Objects | Object Metric(s) |
|---|---|---|
| Whole tumor | 1. EGFR-positive cells | Area density (number of positive cells of each cell type |
| | 2. AREG-Positive cells | over area of ROI) |
| | 3. EREG-Positive cells | Cell ratio (number of positive cells of each cell type |
| | | over total cells in ROI) |
| | | Ligand-receptor ratio (number/density of ligand- |
| | | positive cells (individually or in total) to EGFR- |
| | | positive cell number/density in ROI) |
| Whole tumor | 1. EGFR-positive cells | Area density (number of positive cells of each cell type |
| | 2. AREG-Positive cells | over area of ROI) |
| | within defined distance of at | Cell ratio (number of positive cells of each cell type |
| | least one EGFR-positive | over total cells in ROI) |
| | cell | Ligand-receptor ratio (number/density of ligand- |
| | 3. EREG-Positive cells | positive cells (individually or in total) to EGFR- |
| | within defined distance of at | positive cell number/density in ROI) |
| | least one EGFR-positive | |
| | cell | |
| Whole tumor | 1. Cells with EGFR-positive | Area density (number of positive cells of each cell type |
| | membrane staining | over area of ROI) |
| | 2. AREG-Positive cells | Cell ratio (number of positive cells of each cell type |
| | 3. EREG-Positive cells | over total cells in ROI) |
| | | Ligand-receptor ratio (number/density of ligand- |
| | | positive cells (individually or in total) to |
| | | number/density of Cells with EGFR-positive |
| | | membrane staining in ROI) |
| Whole tumor | 1. Cells with EGFR-positive | Area density (number of positive cells of each cell type |
| | membrane staining | over area of ROI) |
| | 2. AREG-Positive cells | Cell ratio (number of positive cells of each cell type |
| | within defined distance of at | over total cells in ROI) |
| | least one EGFR-positive | Ligand-receptor ratio (number/density of ligand- |
| | cell | positive cells (individually or in total) to |
| | 3. EREG-Positive cells | number/density Cells with EGFR-positive membrane |
| | within defined distance of at | staining in ROI) |
| | least one EGFR-positive | |
| | cell | |
| Whole tumor | 1. Cells with EGFR-positive | Area density (number of positive cells of each cell type |
| | membrane staining | over area of ROI) |
| | 2. Cells with AREG- | Cell ratio (number of positive cells of each cell type |
| | positive membrane staining | over total cells in ROI) |
| | 3. Cells with EREG- | Ligand-receptor ratio (number/density of ligand- |
| | positive membrane staining | positive cells (individually or in total) to |
| | | number/density Cells with EGFR-positive membrane |
| | | staining in ROI) |
| Whole tumor | 1. Cells with EGFR-positive | Area density (number of positive cells of each cell type |
| | membrane staining | over area of ROI) |
| | 2. Cells with AREG- | Cell ratio (number of positive cells of each cell type |
| | positive membrane within | over total cells in ROI) |
| | defined distance of at least | Ligand-receptor ratio (number/density of ligand- |
| | one EGFR-positive cell | positive cells (individually or in total) to EGFR- |
| | 3. Cells with AREG- | positive cell number/density in ROI) |
| | positive membrane within | |
| | defined distance of at least | |
| | one EGFR-positive cell | |
| Whole tumor | 1. EGFR-positive tumor | Area density (number of positive cells of each cell type |
| | cells | over area of ROI) |
| | 2. AREG-Positive cells | Cell ratio (number of positive cells of each cell type |
| | 3. EREG-Positive cells | over total cells in ROI) |
| | | Ligand-receptor ratio (number/density of ligand- |
| | | positive cells (individually or in total) to EGFR- |
| | | positive tumor cell number/density in ROI) |
| Whole tumor | 1. EGFR-positive tumor | Area density (number of positive cells of each cell type |
| | cells | over area of ROI) |
| | 2. AREG-Positive cells | Cell ratio (number of positive cells of each cell type |
| | within defined distance of at | over total cells in ROI) |
| | least one EGFR-positive | Ligand-receptor ratio (number/density of ligand- |
| | cell | positive cells (individually or in total) to EGFR- |
| | 3. EREG-Positive cells | positive tumor cell number/density in ROI) |
| | within defined distance of at | |
| | least one EGFR-positive | |
| | cell | |
| Whole tumor | 1. Tumor cells with EGFR- | Area density (number of positive cells of each cell type |
| | positive membrane staining | over area of ROI) |
| | 2. AREG-Positive cells | Cell ratio (number of positive cells of each cell type |
| | 3. EREG-Positive cells | over total cells in ROI) |
| | | Ligand-receptor ratio (number/density of ligand- |
| | | positive cells (individually or in total) to |
| | | number/density Tumor cells with EGFR-positive |
| | | membrane staining in ROI) |
| Whole tumor | 1. Tumor cells with EGFR- | Area density (number of positive cells of each cell type |
| | positive membrane staining | over area of ROI) |
| | 2. AREG-Positive cells | Cell ratio (number of positive cells of each cell type |
| | within defined distance of at | over total cells in ROI) |
| | least one EGFR-positive | Ligand-receptor ratio (number/density of ligand- |
| | cell | positive cells (individually or in total) to |
| | 3. EREG-Positive cells | number/density Tumor cells with EGFR-positive |
| | within defined distance of at | membrane staining in ROI) |
| | least one EGFR-positive | |
| | cell | |
| Whole tumor | 1. Tumor cells with EGFR- | Area density (number of positive cells of each cell type |
| | positive membrane staining | over area of ROI) |
| | 2. Cells with AREG- | Cell ratio (number of positive cells of each cell type |
| | positive membrane staining | over total cells in ROI) |
| | 3. Cells with EREG- | Ligand-receptor ratio (number/density of ligand- |
| | positive membrane staining | positive cells (individually or in total) to |
| | | number/density Tumor cells with EGFR-positive |
| | | membrane staining in ROI) |
| Whole tumor | 1. Tumor cells with EGFR- | Area density (number of positive cells of each cell type |
| | positive membrane staining | over area of ROI) |
| | 2. Cells with AREG- | Cell ratio (number of positive cells of each cell type |
| | positive membrane within | over total cells in ROI) |
| | defined distance of at least | Ligand-receptor ratio (number/density of ligand- |
| | one EGFR-positive cell | positive cells (individually or in total) to |
| | 3. Cells with AREG- | number/density Tumor cells with EGFR-positive |
| | positive membrane within | membrane staining in ROI) |
| | defined distance of at least | |
| | one tumor cell with EGFR- | |
| | positive membrane staining | |
| Whole tumor | 1. EGFR-positive tumor | Area density (number of positive cells of each cell type |
| | cells | over area of ROI) |
| | 2. AREG-Positive tumor | Cell ratio (number of positive cells of each cell type |
| | cells | over total cells in ROI) |
| | 3. EREG-Positive tumor | Ligand-receptor ratio (number/density of ligand- |
| | cells | positive cells (individually or in total) to EGFR- |
| | | positive tumor cell number/density in ROI) |
| Whole tumor | 1. EGFR-positive tumor | Area density (number of positive cells of each cell type |
| | cells | over area of ROI) |
| | 2. AREG-Positive tumor | Cell ratio (number of positive cells of each cell type |
| | cells within defined distance | over total cells in ROI) |
| | of at least one EGFR- | Ligand-receptor ratio (number/density of ligand- |
| | positive cell | positive cells (individually or in total) to EGFR- |
| | 3. EREG-Positive tumor | positive tumor cell number/density in ROI) |
| | cells within defined distance | |
| | of at least one EGFR- | |
| | positive tumor cell | |
| Whole tumor | 1. Tumor cells with EGFR- | Area density (number of positive cells of each cell type |
| | positive membrane staining | over area of ROI) |
| | 2. AREG-Positive tumor | Cell ratio (number of positive cells of each cell type |
| | cells | over total cells in ROI) |
| | 3. EREG-Positive tumor | Ligand-receptor ratio (number/density of ligand- |
| | cells | positive cells (individually or in total) to |
| | | number/density Tumor cells with EGFR-positive |
| | | membrane staining in ROI) |
| Whole tumor | 1. Tumor cells with EGFR- | Area density (number of positive cells of each cell type |
| | positive membrane staining | over area of ROI) |
| | 2. AREG-Positive tumor | Cell ratio (number of positive cells of each cell type |
| | cells within defined distance | over total cells in ROI) |
| | of at least one EGFR- | Ligand-receptor ratio (number/density of ligand- |
| | positive cell | positive cells (individually or in total) to |
| | 3. EREG-Positive tumor | number/density Tumor cells with EGFR-positive |
| | cells within defined distance | membrane staining in ROI) |
| | of at least one EGFR- | |
| | positive cell | |
| Whole tumor | 1. Tumor cells with EGFR- | Area density (number of positive cells of each cell type |
| | positive membrane staining | over area of ROI) |
| | 2. tumor Cells with AREG- | Cell ratio (number of positive cells of each cell type |
| | positive membrane staining | over total cells in ROI) |
| | 3. tumor Cells with EREG- | Ligand-receptor ratio (number/density of ligand- |
| | positive membrane staining | positive cells (individually or in total) to |
| | | number/density Tumor cells with EGFR-positive |
| | | membrane staining in ROI) |
| Whole tumor | 1. Tumor cells with EGFR- | Area density (number of positive cells of each cell type |
| | positive membrane staining | over area of ROI) |
| | 2. tumor Cells with AREG- | Cell ratio (number of positive cells of each cell type |
| | positive membrane within | over total cells in ROI) |
| | defined distance of at least | Ligand-receptor ratio (number/density of ligand- |
| | one EGFR-positive cell | positive cells (individually or in total) to |
| | 3. tumor Cells with AREG- | number/density tumor cells with EGFR-positive |
| | positive membrane within | membrane staining in ROI) |
| | defined distance of at least | |
| | one EGFR-positive cell | |
| Whole tumor | 1. EGFR-positive | Membrane ratio (ratio of amount/area of each |
| | membrane | membrane type over amount/area of total membrane) |
| | 2. AREG-Positive | Tumor membrane ratio (ratio of amount/area of each |
| | membrane | membrane type over amount/area of total tumor |
| | 3. EREG-Positive | membrane) |
| | membrane | Ligand-receptor membrane ratio (amount/area of |
| | | ligand-positive membrane (individually or in total) to |
| | | amount/area of EGFR-positive membrane) |
| Whole tumor | 1. EGFR-positive | Membrane ratio (ratio of amount/area of each |
| | membrane | membrane type over amount/area of total membrane) |
| | 2. AREG-Positive | Tumor membrane ratio (ratio of amount/area of each |
| | membrane within defined | membrane type over amount/area of total tumor |
| | distance of EGFR-positive | membrane) |
| | tumor membrane | Ligand-receptor membrane ratio (amount/area of |
| | 3. EREG-Positive | ligand-positive membrane (individually or in total) to |
| | membrane within defined | amount/area of EGFR-positive tumor membrane) |
| | distance of EGFR-positive | |
| | tumor membrane | |
| Whole tumor | 1. EGFR-positive tumor | Membrane ratio (ratio of amount/area of each |
| | membrane | membrane type over amount/area of total membrane) |
| | 2. AREG-Positive | Tumor membrane ratio (ratio of amount/area of each |
| | membrane | membrane type over amount/area of total tumor |
| | 3. EREG-Positive | membrane) |
| | membrane | Ligand-receptor membrane ratio (amount/area of |
| | | ligand-positive membrane (individually or in total) to |
| | | amount/area of EGFR-positive tumor membrane) |
| Whole tumor | 1. EGFR-positive tumor | Membrane ratio (ratio of amount/area of each |
| | membrane | membrane type over amount/area of total membrane) |
| | 2. AREG-Positive | Tumor membrane ratio (ratio of amount/area of each |
| | membrane within defined | membrane type over amount/area of total tumor |
| | distance of EGFR-positive | membrane) |
| | tumor membrane | Ligand-receptor membrane ratio (amount/area of |
| | 3. EREG-Positive | ligand-positive membrane (individually or in total) to |
| | membrane within defined | amount/area of EGFR-positive tumor membrane) |
| | distance of EGFR-positive | |
| | tumor membrane | |
| Whole tumor | 1. EGFR-positive tumor | Membrane ratio (ratio of amount/area of each |
| | membrane | membrane type over amount/area of total membrane) |
| | 2. AREG-Positive tumor | Tumor membrane ratio (ratio of amount/area of each |
| | membrane | membrane type over amount/area of total tumor |
| | 3. EREG-Positive tumor | membrane) |
| | membrane | Ligand-receptor membrane ratio (amount/area of |
| | | ligand-positive tumor membrane (individually or in |
| | | total) to amount/area of EGFR-positive tumor |
| | | membrane) |
| Whole tumor | 1. EGFR-positive tumor | Membrane ratio (ratio of amount/area of each |
| | membrane | membrane type over amount/area of total membrane) |
| | 2. AREG-Positive tumor | Tumor membrane ratio (ratio of amount/area of each |
| | membrane within defined | membrane type over amount/area of total tumor |
| | distance of EGFR-positive | membrane) |
| | tumor membrane | Ligand-receptor membrane ratio (amount/area of |
| | 3. EREG-Positive tumor | ligand-positive tumor membrane (individually or in |
| | membrane within defined | total) to amount/area of EGFR-positive tumor |
| | distance of EGFR-positive | membrane) |
| | tumor membrane | |
| EGFR-positive | 1. EGFR-positive cells | Area density (number of positive cells of each cell type |
| cell cluster | 2. AREG-Positive cells | over area of ROI) |
| | 3. EREG-Positive cells | Cell ratio (number of positive cells of each cell type |
| | | over total cells in ROI) |
| | | Ligand-receptor ratio (number/density of ligand- |
| | | positive cells (individually or in total) to EGFR- |
| | | positive cell number/density in ROI) |
| EGFR-positive | 1. EGFR-positive cells | Area density (number of positive cells of each cell type |
| cell cluster | 2. AREG-Positive cells | over area of ROI) |
| | within defined distance of at | Cell ratio (number of positive cells of each cell type |
| | least one EGFR-positive | over total cells in ROI) |
| | cell | Ligand-receptor ratio (number/density of ligand- |
| | 3. EREG-Positive cells | positive cells (individually or in total) to EGFR- |
| | within defined distance of at | positive cell number/density in ROI) |
| | least one EGFR-positive | |
| | cell | |
| EGFR-positive | 1. Cells with EGFR-positive | Area density (number of positive cells of each cell type |
| cell cluster | membrane staining | over area of ROI) |
| | 2. AREG-Positive cells | Cell ratio (number of positive cells of each cell type |
| | 3. EREG-Positive cells | over total cells in ROI) |
| | | Ligand-receptor ratio (number/density of ligand- |
| | | positive cells (individually or in total) to |
| | | number/density of Cells with EGFR-positive |
| | | membrane staining in ROI) |
| EGFR-positive | 1. Cells with EGFR-positive | Area density (number of positive cells of each cell type |
| cell cluster | membrane staining | over area of ROI) |
| | 2. AREG-Positive cells | Cell ratio (number of positive cells of each cell type |
| | within defined distance of at | over total cells in ROI) |
| | least one EGFR-positive | Ligand-receptor ratio (number/density of ligand- |
| | cell | positive cells (individually or in total) to |
| | 3. EREG-Positive cells | number/density Cells with EGFR-positive membrane |
| | within defined distance of at | staining in ROI) |
| | least one EGFR-positive | |
| | cell | |
| EGFR-positive | 1. Cells with EGFR-positive | Area density (number of positive cells of each cell type |
| cell cluster | membrane staining | over area of ROI) |
| | 2. Cells with AREG- | Cell ratio (number of positive cells of each cell type |
| | positive membrane staining | over total cells in ROI) |
| | 3. Cells with EREG- | Ligand-receptor ratio (number/density of ligand- |
| | positive membrane staining | positive cells (individually or in total) to |
| | | number/density Cells with EGFR-positive membrane |
| | | staining in ROI) |
| EGFR-positive | 1. Cells with EGFR-positive | Area density (number of positive cells of each cell type |
| cell cluster | membrane staining | over area of ROI) |
| | 2. Cells with AREG- | Cell ratio (number of positive cells of each cell type |
| | positive membrane within | over total cells in ROI) |
| | defined distance of at least | Ligand-receptor ratio (number/density of ligand- |
| | one EGFR-positive cell | positive cells (individually or in total) to EGFR- |
| | 3. Cells with AREG- | positive cell number/density in ROI) |
| | positive membrane within | |
| | defined distance of at least | |
| | one EGFR-positive cell | |
| EGFR-positive | 1. EGFR-positive tumor | Area density (number of positive cells of each cell type |
| cell cluster | cells | over area of ROI) |
| | 2. AREG-Positive cells | Cell ratio (number of positive cells of each cell type |
| | 3. EREG-Positive cells | over total cells in ROI) |
| | | Ligand-receptor ratio (number/density of ligand- |
| | | positive cells (individually or in total) to EGFR- |
| | | positive tumor cell number/density in ROI) |
| EGFR-positive | 1. EGFR-positive tumor | Area density (number of positive cells of each cell type |
| cell cluster | cells | over area of ROI) |
| | 2. AREG-Positive cells | Cell ratio (number of positive cells of each cell type |
| | within defined distance of at | over total cells in ROI) |
| | least one EGFR-positive | Ligand-receptor ratio (number/density of ligand- |
| | cell | positive cells (individually or in total) to EGFR- |
| | 3. EREG-Positive cells | positive tumor cell number/density in ROI) |
| | within defined distance of at | |
| | least one EGFR-positive | |
| | cell | |
| EGFR-positive | 1. Tumor cells with EGFR- | Area density (number of positive cells of each cell type |
| cell cluster | positive membrane staining | over area of ROI) |
| | 2. AREG-Positive cells | Cell ratio (number of positive cells of each cell type |
| | 3. EREG-Positive cells | over total cells in ROI) |
| | | Ligand-receptor ratio (number/density of ligand- |
| | | positive cells (individually or in total) to |
| | | number/density Tumor cells with EGFR-positive |
| | | membrane staining in ROI) |
| EGFR-positive | 1. Tumor cells with EGFR- | Area density (number of positive cells of each cell type |
| cell cluster | positive membrane staining | over area of ROI) |
| | 2. AREG-Positive cells | Cell ratio (number of positive cells of each cell type |
| | within defined distance of at | over total cells in ROI) |
| | least one EGFR-positive | Ligand-receptor ratio (number/density of ligand- |
| | cell | positive cells (individually or in total) to |
| | 3. EREG-Positive cells | number/density Tumor cells with EGFR-positive |
| | within defined distance of at | membrane staining in ROI) |
| | least one EGFR-positive | |
| | cell | |
| EGFR-positive | 1. Tumor cells with EGFR- | Area density (number of positive cells of each cell type |
| cell cluster | positive membrane staining | over area of ROI) |
| | 2. Cells with AREG- | Cell ratio (number of positive cells of each cell type |
| | positive membrane staining | over total cells in ROI) |
| | 3. Cells with EREG- | Ligand-receptor ratio (number/density of ligand- |
| | positive membrane staining | positive cells (individually or in total) to |
| | | number/density Tumor cells with EGFR-positive |
| | | membrane staining in ROI) |
| EGFR-positive | 1. Tumor cells with EGFR- | Area density (number of positive cells of each cell type |
| cell cluster | positive membrane staining | over area of ROI) |
| | 2. Cells with AREG- | Cell ratio (number of positive cells of each cell type |
| | positive membrane within | over total cells in ROI) |
| | defined distance of at least | Ligand-receptor ratio (number/density of ligand- |
| | one EGFR-positive cell | positive cells (individually or in total) to |
| | 3. Cells with AREG- | number/density Tumor cells with EGFR-positive |
| | positive membrane within | membrane staining in ROI) |
| | defined distance of at least | |
| | one tumor cell with EGFR- | |
| | positive membrane staining | |
| EGFR-positive | 1. EGFR-positive tumor | Area density (number of positive cells of each cell type |
| cell cluster | cells | over area of ROI) |
| | 2. AREG-Positive tumor | Cell ratio (number of positive cells of each cell type |
| | cells | over total cells in ROI) |
| | 3. EREG-Positive tumor | Ligand-receptor ratio (number/density of ligand- |
| | cells | positive cells (individually or in total) to EGFR- |
| | | positive tumor cell number/density in ROI) |
| EGFR-positive | 1. EGFR-positive tumor | Area density (number of positive cells of each cell type |
| cell cluster | cells | over area of ROI) |
| | 2. AREG-Positive tumor | Cell ratio (number of positive cells of each cell type |
| | cells within defined distance | over total cells in ROI) |
| | of at least one EGFR- | Ligand-receptor ratio (number/density of ligand- |
| | positive cell | positive cells (individually or in total) to EGFR- |
| | 3. EREG-Positive tumor | positive tumor cell number/density in ROI) |
| | cells within defined distance | |
| | of at least one EGFR- | |
| | positive tumor cell | |
| EGFR-positive | 1. Tumor cells with EGFR- | Area density (number of positive cells of each cell type |
| cell cluster | positive membrane staining | over area of ROI) |
| | 2. AREG-Positive tumor | Cell ratio (number of positive cells of each cell type |
| | cells | over total cells in ROI) |
| | 3. EREG-Positive tumor | Ligand-receptor ratio (number/density of ligand- |
| | cells | positive cells (individually or in total) to |
| | | number/density Tumor cells with EGFR-positive |
| | | membrane staining in ROI) |
| EGFR-positive | 1. Tumor cells with EGFR- | Area density (number of positive cells of each cell type |
| cell cluster | positive membrane staining | over area of ROI) |
| | 2. AREG-Positive tumor | Cell ratio (number of positive cells of each cell type |
| | cells within defined distance | over total cells in ROI) |
| | of at least one EGFR- | Ligand-receptor ratio (number/density of ligand- |
| | positive cell | positive cells (individually or in total) to |
| | 3. EREG-Positive tumor | number/density Tumor cells with EGFR-positive |
| | cells within defined distance | membrane staining in ROI) |
| | of at least one EGFR- | |
| | positive cell | |
| EGFR-positive | 1. Tumor cells with EGFR- | Area density (number of positive cells of each cell type |
| cell cluster | positive membrane staining | over area of ROI) |
| | 2. tumor Cells with AREG- | Cell ratio (number of positive cells of each cell type |
| | positive membrane staining | over total cells in ROI) |
| | 3. tumor Cells with EREG- | Ligand-receptor ratio (number/density of ligand- |
| | positive membrane staining | positive cells (individually or in total) to |
| | | number/density Tumor cells with EGFR-positive |
| | | membrane staining in ROI) |
| EGFR-positive | 1. Tumor cells with EGFR- | Area density (number of positive cells of each cell type |
| cell cluster | positive membrane staining | over area of ROI) |
| | 2. tumor Cells with AREG- | Cell ratio (number of positive cells of each cell type |
| | positive membrane within | over total cells in ROI) |
| | defined distance of at least | Ligand-receptor ratio (number/density of ligand- |
| | one EGFR-positive cell | positive cells (individually or in total) to |
| | 3. tumor Cells with AREG- | number/density tumor cells with EGFR-positive |
| | positive membrane within | membrane staining in ROI) |
| | defined distance of at least | |
| | one EGFR-positive cell | |
| EGFR-positive | 1. EGFR-positive | Membrane ratio (ratio of amount/area of each |
| cell cluster | membrane | membrane type over amount/area of total membrane) |
| | 2. AREG-Positive | Tumor membrane ratio (ratio of amount/area of each |
| | membrane | membrane type over amount/area of total tumor |
| | 3. EREG-Positive | membrane) |
| | membrane | Ligand-receptor membrane ratio (amount/area of |
| | | ligand-positive membrane (individually or in total) to |
| | | amount/area of EGFR-positive membrane) |
| EGFR-positive | 1. EGFR-positive | Membrane ratio (ratio of amount/area of each |
| cell cluster | membrane | membrane type over amount/area of total membrane) |
| | 2. AREG-Positive | Tumor membrane ratio (ratio of amount/area of each |
| | membrane within defined | membrane type over amount/area of total tumor |
| | distance of EGFR-positive | membrane) |
| | tumor membrane | Ligand-receptor membrane ratio (amount/area of |
| | 3. EREG-Positive | ligand-positive membrane (individually or in total) to |
| | membrane within defined | amount/area of EGFR-positive tumor membrane) |
| | distance of EGFR-positive | |
| | tumor membrane | |
| EGFR-positive | 1. EGFR-positive tumor | Membrane ratio (ratio of amount/area of each |
| cell cluster | membrane | membrane type over amount/area of total membrane) |
| | 2. AREG-Positive | Tumor membrane ratio (ratio of amount/area of each |
| | membrane | membrane type over amount/area of total tumor |
| | 3. EREG-Positive | membrane) |
| | membrane | Ligand-receptor membrane ratio (amount/area of |
| | | ligand-positive membrane (individually or in total) to |
| | | amount/area of EGFR-positive tumor membrane) |
| EGFR-positive | 1. EGFR-positive tumor | Membrane ratio (ratio of amount/area of each |
| cell cluster | membrane | membrane type over amount/area of total membrane) |
| | 2. AREG-Positive | Tumor membrane ratio (ratio of amount/area of each |
| | membrane within defined | membrane type over amount/area of total tumor |
| | distance of EGFR-positive | membrane) |
| | tumor membrane | Ligand-receptor membrane ratio (amount/area of |
| | 3. EREG-Positive | ligand-positive membrane (individually or in total) to |
| | membrane within defined | amount/area of EGFR-positive tumor membrane) |
| | distance of EGFR-positive | |
| | tumor membrane | |
| EGFR-positive | 1. EGFR-positive tumor | Membrane ratio (ratio of amount/area of each |
| cell cluster | membrane | membrane type over amount/area of total membrane) |
| | 2. AREG-Positive tumor | Tumor membrane ratio (ratio of amount/area of each |
| | membrane | membrane type over amount/area of total tumor |
| | 3. EREG-Positive tumor | membrane) |
| | membrane | Ligand-receptor membrane ratio (amount/area of |
| | | ligand-positive tumor membrane (individually or in |
| | | total) to amount/area of EGFR-positive tumor |
| | | membrane) |
| EGFR-positive | 1. EGFR-positive tumor | Membrane ratio (ratio of amount/area of each |
| cell cluster | membrane | membrane type over amount/area of total membrane) |
| | 2. AREG-Positive tumor | Tumor membrane ratio (ratio of amount/area of each |
| | membrane within defined | membrane type over amount/area of total tumor |
| | distance of EGFR-positive | membrane) |
| | tumor membrane | Ligand-receptor membrane ratio (amount/area of |
| | 3. EREG-Positive tumor | ligand-positive tumor membrane (individually or in |
| | membrane within defined | total) to amount/area of EGFR-positive tumor |
| | distance of EGFR-positive | membrane) |
| | tumor membrane | |
| Points within | 1. EGFR-positive cells | Area density (number of positive cells of each cell type |
| defined distance | 2. AREG-Positive cells | over area of ROI) |
| of EGFR-positive | 3. EREG-Positive cells | Cell ratio (number of positive cells of each cell type |
| cell cluster | | over total cells in ROI) |
| | | Ligand-receptor ratio (number/density of ligand- |
| | | positive cells (individually or in total) to EGFR- |
| | | positive cell number/density in ROI) |
| Points within | 1. EGFR-positive cells | Area density (number of positive cells of each cell type |
| defined distance | 2. AREG-Positive cells | over area of ROI) |
| of EGFR-positive | within defined distance of at | Cell ratio (number of positive cells of each cell type |
| cell cluster | least one EGFR-positive | over total cells in ROI) |
| | cell | Ligand-receptor ratio (number/density of ligand- |
| | 3. EREG-Positive cells | positive cells (individually or in total) to EGFR- |
| | within defined distance of at | positive cell number/density in ROI) |
| | least one EGFR-positive | |
| | cell | |
| Points within | 1. Cells with EGFR-positive | Area density (number of positive cells of each cell type |
| defined distance | membrane staining | over area of ROI) |
| of EGFR-positive | 2. AREG-Positive cells | Cell ratio (number of positive cells of each cell type |
| cell cluster | 3. EREG-Positive cells | over total cells in ROI) |
| | | Ligand-receptor ratio (number/density of ligand- |
| | | positive cells (individually or in total) to |
| | | number/density of Cells with EGFR-positive |
| | | membrane staining in ROI) |
| Points within | 1. Cells with EGFR-positive | Area density (number of positive cells of each cell type |
| defined distance | membrane staining | over area of ROI) |
| of EGFR-positive | 2. AREG-Positive cells | Cell ratio (number of positive cells of each cell type |
| cell cluster | within defined distance of at | over total cells in ROI) |
| | least one EGFR-positive | Ligand-receptor ratio (number/density of ligand- |
| | cell | positive cells (individually or in total) to |
| | 3. EREG-Positive cells | number/density Cells with EGFR-positive membrane |
| | within defined distance of at | staining in ROI) |
| | least one EGFR-positive | |
| | cell | |
| Points within | 1. Cells with EGFR-positive | Area density (number of positive cells of each cell type |
| defined distance | membrane staining | over area of ROI) |
| of EGFR-positive | 2. Cells with AREG- | Cell ratio (number of positive cells of each cell type |
| cell cluster | positive membrane staining | over total cells in ROI) |
| | 3. Cells with EREG- | Ligand-receptor ratio (number/density of ligand- |
| | positive membrane staining | positive cells (individually or in total) to |
| | | number/density Cells with EGFR-positive membrane |
| | | staining in ROI) |
| Points within | 1. Cells with EGFR-positive | Area density (number of positive cells of each cell type |
| defined distance | membrane staining | over area of ROI) |
| of EGFR-positive | 2. Cells with AREG- | Cell ratio (number of positive cells of each cell type |
| cell cluster | positive membrane within | over total cells in ROI) |
| | defined distance of at least | Ligand-receptor ratio (number/density of ligand- |
| | one EGFR-positive cell | positive cells (individually or in total) to EGFR- |
| | 3. Cells with AREG- | positive cell number/density in ROI) |
| | positive membrane within | |
| | defined distance of at least | |
| | one EGFR-positive cell | |
| Points within | 1. EGFR-positive tumor | Area density (number of positive cells of each cell type |
| defined distance | cells | over area of ROI) |
| of EGFR-positive | 2. AREG-Positive cells | Cell ratio (number of positive cells of each cell type |
| cell cluster | 3. EREG-Positive cells | over total cells in ROI) |
| | | Ligand-receptor ratio (number/density of ligand- |
| | | positive cells (individually or in total) to EGFR- |
| | | positive tumor cell number/density in ROI) |
| Points within | 1. EGFR-positive tumor | Area density (number of positive cells of each cell type |
| defined distance | cells | over area of ROI) |
| of EGFR-positive | 2. AREG-Positive cells | Cell ratio (number of positive cells of each cell type |
| cell cluster | within defined distance of at | over total cells in ROI) |
| | least one EGFR-positive | Ligand-receptor ratio (number/density of ligand- |
| | cell | positive cells (individually or in total) to EGFR- |
| | 3. EREG-Positive cells | positive tumor cell number/density in ROI) |
| | within defined distance of at | |
| | least one EGFR-positive | |
| | cell | |
| Points within | 1. Tumor cells with EGFR- | Area density (number of positive cells of each cell type |
| defined distance | positive membrane staining | over area of ROI) |
| of EGFR-positive | 2. AREG-Positive cells | Cell ratio (number of positive cells of each cell type |
| cell cluster | 3. EREG-Positive cells | over total cells in ROI) |
| | | Ligand-receptor ratio (number/density of ligand- |
| | | positive cells (individually or in total) to |
| | | number/density Tumor cells with EGFR-positive |
| | | membrane staining in ROI) |
| Points within | 1. Tumor cells with EGFR- | Area density (number of positive cells of each cell type |
| defined distance | positive membrane staining | over area of ROI) |
| of EGFR-positive | 2. AREG-Positive cells | Cell ratio (number of positive cells of each cell type |
| cell cluster | within defined distance of at | over total cells in ROI) |
| | least one EGFR-positive | Ligand-receptor ratio (number/density of ligand- |
| | cell | positive cells (individually or in total) to |
| | 3. EREG-Positive cells | number/density Tumor cells with EGFR-positive |
| | within defined distance of at | membrane staining in ROI) |
| | least one EGFR-positive | |
| | cell | |
| Points within | 1. Tumor cells with EGFR- | Area density (number of positive cells of each cell type |
| defined distance | positive membrane staining | over area of ROI) |
| of EGFR-positive | 2. Cells with AREG- | Cell ratio (number of positive cells of each cell type |
| cell cluster | positive membrane staining | over total cells in ROI) |
| | 3. Cells with EREG- | Ligand-receptor ratio (number/density of ligand- |
| | positive membrane staining | positive cells (individually or in total) to |
| | | number/density Tumor cells with EGFR-positive |
| | | membrane staining in ROI) |
| Points within | 1. Tumor cells with EGFR- | Area density (number of positive cells of each cell type |
| defined distance | positive membrane staining | over area of ROI) |
| of EGFR-positive | 2. Cells with AREG- | Cell ratio (number of positive cells of each cell type |
| cell cluster | positive membrane within | over total cells in ROI) |
| | defined distance of at least | Ligand-receptor ratio (number/density of ligand- |
| | one EGFR-positive cell | positive cells (individually or in total) to |
| | 3. Cells with AREG- | number/density Tumor cells with EGFR-positive |
| | positive membrane within | membrane staining in ROI) |
| | defined distance of at least | |
| | one tumor cell with EGFR- | |
| | positive membrane staining | |
| Points within | 1. EGFR-positive tumor | Area density (number of positive cells of each cell type |
| defined distance | cells | over area of ROI) |
| of EGFR-positive | 2. AREG-Positive tumor | Cell ratio (number of positive cells of each cell type |
| cell cluster | cells | over total cells in ROI) |
| | 3. EREG-Positive tumor | Ligand-receptor ratio (number/density of ligand- |
| | cells | positive cells (individually or in total) to EGFR- |
| | | positive tumor cell number/density in ROI) |
| Points within | 1. EGFR-positive tumor | Area density (number of positive cells of each cell type |
| defined distance | cells | over area of ROI) |
| of EGFR-positive | 2. AREG-Positive tumor | Cell ratio (number of positive cells of each cell type |
| cell cluster | cells within defined distance | over total cells in ROI) |
| | of at least one EGFR- | Ligand-receptor ratio (number/density of ligand- |
| | positive cell | positive cells (individually or in total) to EGFR- |
| | 3. EREG-Positive tumor | positive tumor cell number/density in ROI) |
| | cells within defined distance | |
| | of at least one EGFR- | |
| | positive tumor cell | |
| Points within | 1. Tumor cells with EGFR- | Area density (number of positive cells of each cell type |
| defined distance | positive membrane staining | over area of ROI) |
| of EGFR-positive | 2. AREG-Positive tumor | Cell ratio (number of positive cells of each cell type |
| cell cluster | cells | over total cells in ROI) |
| | 3. EREG-Positive tumor | Ligand-receptor ratio (number/density of ligand- |
| | cells | positive cells (individually or in total) to |
| | | number/density Tumor cells with EGFR-positive |
| | | membrane staining in ROI) |
| Points within | 1. Tumor cells with EGFR- | Area density (number of positive cells of each cell type |
| defined distance | positive membrane staining | over area of ROI) |
| of EGFR-positive | 2. AREG-Positive tumor | Cell ratio (number of positive cells of each cell type |
| cell cluster | cells within defined distance | over total cells in ROI) |
| | of at least one EGFR- | Ligand-receptor ratio (number/density of ligand- |
| | positive cell | positive cells (individually or in total) to |
| | 3. EREG-Positive tumor | number/density Tumor cells with EGFR-positive |
| | cells within defined distance | membrane staining in ROI) |
| | of at least one EGFR- | |
| | positive cell | |
| Points within | 1. Tumor cells with EGFR- | Area density (number of positive cells of each cell type |
| defined distance | positive membrane staining | over area of ROI) |
| of EGFR-positive | 2. tumor Cells with AREG- | Cell ratio (number of positive cells of each cell type |
| cell cluster | positive membrane staining | over total cells in ROI) |
| | 3. tumor Cells with EREG- | Ligand-receptor ratio (number/density of ligand- |
| | positive membrane staining | positive cells (individually or in total) to |
| | | number/density Tumor cells with EGFR-positive |
| | | membrane staining in ROI) |
| Points within | 1. Tumor cells with EGFR- | Area density (number of positive cells of each cell type |
| defined distance | positive membrane staining | over area of ROI) |
| of EGFR-positive | 2. tumor Cells with AREG- | Cell ratio (number of positive cells of each cell type |
| cell cluster | positive membrane within | over total cells in ROI) |
| | defined distance of at least | Ligand-receptor ratio (number/density of ligand- |
| | one EGFR-positive cell | positive cells (individually or in total) to |
| | 3. tumor Cells with AREG- | number/density tumor cells with EGFR-positive |
| | positive membrane within | membrane staining in ROI) |
| | defined distance of at least | |
| | one EGFR-positive cell | |
| Points within | 1. EGFR-positive | Membrane ratio (ratio of amount/area of each |
| defined distance | membrane | membrane type over amount/area of total membrane) |
| of EGFR-positive | 2. AREG-Positive | Tumor membrane ratio (ratio of amount/area of each |
| cell cluster | membrane | membrane type over amount/area of total tumor |
| | 3. EREG-Positive | membrane) |
| | membrane | Ligand-receptor membrane ratio (amount/area of |
| | | ligand-positive membrane (individually or in total) to |
| | | amount/area of EGFR-positive membrane) |
| Points within | 1. EGFR-positive | Membrane ratio (ratio of amount/area of each |
| defined distance | membrane | membrane type over amount/area of total membrane) |
| of EGFR-positive | 2. AREG-Positive | Tumor membrane ratio (ratio of amount/area of each |
| cell cluster | membrane within defined | membrane type over amount/area of total tumor |
| | distance of EGFR-positive | membrane) |
| | tumor membrane | Ligand-receptor membrane ratio (amount/area of |
| | 3. EREG-Positive | ligand-positive membrane (individually or in total) to |
| | membrane within defined | amount/area of EGFR-positive tumor membrane) |
| | distance of EGFR-positive | |
| | tumor membrane | |
| Points within | 1. EGFR-positive tumor | Membrane ratio (ratio of amount/area of each |
| defined distance | membrane | membrane type over amount/area of total membrane) |
| of EGFR-positive | 2. AREG-Positive | Tumor membrane ratio (ratio of amount/area of each |
| cell cluster | membrane | membrane type over amount/area of total tumor |
| | 3. EREG-Positive | membrane) |
| | membrane | Ligand-receptor membrane ratio (amount/area of |
| | | ligand-positive membrane (individually or in total) to |
| | | amount/area of EGFR-positive tumor membrane) |
| Points within | 1. EGFR-positive tumor | Membrane ratio (ratio of amount/area of each |
| defined distance | membrane | membrane type over amount/area of total membrane) |
| of EGFR-positive | 2. AREG-Positive | Tumor membrane ratio (ratio of amount/area of each |
| cell cluster | membrane within defined | membrane type over amount/area of total tumor |
| | distance of EGFR-positive | membrane) |
| | tumor membrane | Ligand-receptor membrane ratio (amount/area of |
| | 3. EREG-Positive | ligand-positive membrane (individually or in total) to |
| | membrane within defined | amount/area of EGFR-positive tumor membrane) |
| | distance of EGFR-positive | |
| | tumor membrane | |
| Points within | 1. EGFR-positive tumor | Membrane ratio (ratio of amount/area of each |
| defined distance | membrane | membrane type over amount/area of total membrane) |
| of EGFR-positive | 2. AREG-Positive tumor | Tumor membrane ratio (ratio of amount/area of each |
| cell cluster | membrane | membrane type over amount/area of total tumor |
| | 3. EREG-Positive tumor | membrane) |
| | membrane | Ligand-receptor membrane ratio (amount/area of |
| | | ligand-positive tumor membrane (individually or in |
| | | total) to amount/area of EGFR-positive tumor |
| | | membrane) |
| Points within | 1. EGFR-positive tumor | Membrane ratio (ratio of amount/area of each |
| defined distance | membrane | membrane type over amount/area of total membrane) |
| of EGFR-positive | 2. AREG-Positive tumor | Tumor membrane ratio (ratio of amount/area of each |
| cell cluster | membrane within defined | membrane type over amount/area of total tumor |
| | distance of EGFR-positive | membrane) |
| | tumor membrane | Ligand-receptor membrane ratio (amount/area of |
| | 3. EREG-Positive tumor | ligand-positive tumor membrane (individually or in |
| | membrane within defined | total) to amount/area of EGFR-positive tumor |
| | distance of EGFR-positive | membrane) |
| | tumor membrane | |

If desired, the calculated object metrics optionally may be converted to a normalized feature vector. In the typical example, the object metrics calculated for the samples of the cohort are plotted, and the distribution is evaluated to identify any rightward or leftward skew. Biologically meaningful cutoffs (maximum cutoffs for right-skewed distributions, and/or minimum cutoffs for left-skewed distributions) are identified, and each sample having a value beyond the cutoff (above in the case of a right-skewed distribution, or below in the case of left-skewed distribution) is assigned an object metric equal to the cutoff value. The cutoff value (hereafter referred to as the “normalization factor”) is then applied to each object metric. In the case of a right-skewed distribution, the object metric is divided by the normalization factor to obtain the normalized object metric, in which case the object metric is expressed on a maximum scale (i.e. the value of the normalized metric will not exceed a pre-determined maximum, such as 1, 10, 100, etc.). Similarly, in the case of a left-skewed distribution, the object metric is divided by the normalization factor to obtain the normalized object metric, in which case the object metric is expressed on a minimum scale (i.e. the value of the normalized metric will not fall below a pre-determined minimum, such as 1, 10, 100, etc.). If desired, the normalized object metric may also be multiplied by or divided by a pre-determined constant value to obtain the desired scale (for example, for right skewed distributions, multiplied by 100 to obtain a percentage of the normalization factor instead of a fraction of the normalization factor). Normalized object metrics may be calculated for test samples by applying the normalization factor and/or maximum and/or minimum cutoffs identified for modeling to the object metric calculated for the test sample.

In another embodiment, the objects are clustered into one of a plurality groups on the basis of various extracted features, such as, for example, cell size, shape, staining intensity, texture, staining response, etc. In an exemplary embodiment, an unsupervised clustering function is applied to the image, such as the function described in U.S. 62/441,068, filed Dec. 30, 2016

C. Scoring Function

In an embodiment in which a prediction of response to EGFR therapy is desired, a scoring engine may be implemented. The scoring engine applies a scoring function to a feature vector comprising the object metrics for each of the biomarkers being evaluated and calculates a score. The scoring engine may then generate a report including the score.

In order to identify the scoring function, object metrics of a cohort of patients with known outcomes are modeled for their ability to predict the relative tumor prognosis, risk of progression, and/or likelihood of responding to a particular treatment course.

In an embodiment, the scoring function is derived by modeling various combinations of object metrics for their correlation with various outcome events. The object metrics for the samples may be modeled against the outcomes using a one or more of a variety of models, including “time-to-event” models (such as Cox proportional hazard models for overall survival, progression-free survival, or recurrence-free survival) and binary event models (such as logistic regression models). In an embodiment, a “time-to-event” model is used. These models test each variable for the ability to predict the relative risk of a defined event occurring at any given time point. The “event” in such a case is typically overall survival, recurrence-free survival, and/or progression-free survival. In one example, the “time-to event” model is a Cox proportional hazard model for overall survival, recurrence-free survival, or progression-free survival. The Cox proportional hazard model can be written as formula 1:

Score=exp(b₁X₁+b₂X₂+ . . . bₚXₚ) Formula 1

in each case, wherein X₁, X₂, . . . Xp are the values of the object metric(s) (which optionally may be subject to maximum and/or minimum cutoffs, and/or normalization), b₁, b₂ . . . bₚ are constants extrapolated from the model for each of the feature metric(s). For each patient sample of the test cohort, data is obtained regarding the outcome being tracked (time to death, time to recurrence, or time to progression) and the feature metric for each biomarker being analyzed. Candidate Cox proportional models are generated by entering the feature metric data and survival data for each individual of the cohort into a computerized statistical analysis software suite (such as The R Project for Statistical Computing (available at https://www.r-project.org/), SAS, MATLAB, among others). Each candidate model is tested for predictive ability using a concordance index, such as C-index. The model having the highest concordance score using the selected concordance index is selected as the continuous scoring function.

Additionally, one or more stratification cutoffs may be selected to separate the patients into “risk bins” according to relative risk (such as “high risk” and “low risk,” quartiles, deciles, etc.). In one example, stratification cutoffs are selected using receiver operator characteristic (ROC) curves. ROC curves allow users to balance the sensitivity of the model (i.e. prioritize capturing as many “positive” or “high risk” candidates as possible) with the specificity of the model (i.e. minimizing false-positives for “high risk candidates”). In an embodiment, a cutoff between high risk and low risk bins for overall survival, recurrence-free survival or progression-free survival is selected, the cutoff chosen having the sensitivity and specificity balanced.

After the scoring function has been modeled and optional stratification cutoffs have been selected, the scoring function may be applied to images of test samples to calculate a response score for the test sample. The test samples are typically similar to the sample types used for modeling the continuous scoring function, except that outcomes are not yet known. The test samples are stained for the biomarkers relevant to the scoring function and the relevant object metrics are calculated, and if they are being used, the normalization factor(s) and/or maximum and/or minimum cutoffs are applied to the feature metrics to obtain the normalized feature metrics. The response score is calculated by applying the scoring function to the feature metrics or the normalized feature metrics. The response score may then be integrated into diagnostic and/or treatment decisions by a clinician.

### IV. CLINICAL APPLICATIONS

In clinical practice, the score obtained from the histochemical staining as described above may be used to determine a course of treatment for a patient. The present disclosure also feature methods of treating patients with an anti-EGFR therapy wherein the patient is treated with the anti-EGFR therapy if he/she has a tumor that is scored or categorized (as above) as a “predicted positive response to an anti-EGFR therapy” or a “likely to respond to an anti-EGFR therapy.”

In an embodiment, the anti-EGFR therapy is an EGFR antibody-based therapy. These therapies typically rely on antibodies or antibody fragments that bind to an extracellular domain of EGFR and disrupt association between EGFR and its ligands (including EREG and AREG). In an embodiment, the EGFR antibody-based therapy comprises cetuximab and/or panitumumab. In an embodiment, an EGFR antibody-based therapy is administered if: (a) the expression pattern of EGFR and one or more of EREG and AREG indicates that the patient is likely to respond to the EGFR antibody-based therapy; and (b) the subject or sample is determined to be RAS wild-type. Ras proteins are small GTPases active as downstream components of the EGFR signaling network. Human Ras proteins are encoded by one of three RAS genes: HRAS (encoding h-Ras protein), KRAS (encoding k-Ras protein), and NRAS (encoding n-Ras protein). HRAS, KRAS, and NRAS genes are collectively referred to herein as “RAS.” H-Ras, k-Ras, and n-Ras proteins are collectively referred to herein as “Ras protein.” A canonical sequences for human h-Ras protein is provided at SEQ ID NO: 4 (Uniprot Accession No. P01112-1). A canonical sequence for human k-Ras protein is provided as SEQ ID NO: 5 (Uniprot Accession No. P01116-1). A canonical sequence for human n-Ras protein is provided at SEQ ID NO: 6 (Uniprot Accession No. P01111-1). Oncogenic mutations of RAS typically result in constitutively active forms of Ras protein. Thus, patients with activating mutations in at least one Ras protein are likely to be resistant to anti-EGFR therapies. Activating Ras mutations in colorectal cancer are reviewed by Prior et al., Cancer Res. Vol. 72, Issue 10, pp. 2457-67 (May 2012) (incorporated by reference), and Waring et al., Clin. Colorectal Cancer, Vol. 15, Issue 2, pp. 95-103 (June 2016) (incorporated by reference), among others. As used herein, a “wild-type RAS” shall mean that the sample or subject has tested negative in a RAS mutation screening assay for mutations within at least NRAS and KRAS that confer resistance to EGFR monoclonal antibody therapy (whether currently known or later discovered). In an embodiment, the RAS mutation screening assay comprises determining the presence or absence of activating mutations in at least codons 12 and 13 of NRAS and codons 12 and 13 of KRAS, wherein the sample is considered “RAS wild type” if the samples or subject is free of activating mutations of each of codons 12 and 13 of NRAS and codons 12 and 13 of KRAS. In another embodiment, the RAS mutation screening assay comprises determining the presence or absence of activating mutations in at least codons 12, 13, 59, 61, 117, and 146 of NRAS and codons 12, 13, 59, 61, 117, and 146 of KRAS, wherein the sample is considered “RAS wild type” if the samples or subject is free of activating mutations of each of codons 12, 13, 59, 61, 117, and 146 of NRAS and codons 12, 13, 59, 61, 117, and 146 of KRAS are determined to have wild-type RAS status. Screening for Ras mutation status may be performed on a variety of different types of samples from the subject, including tissue samples derived from the tumor and blood samples from the same subject from which the tissue sample has been obtained. Many different methods for screening for Ras mutational status are known, including methods based on sequencing, pyrosequencing, real-time PCR, allele-specific real-time PCR, Restriction fragment length polymorphism (RFLP) analysis with sequencing, amplification refractory mutation systems (ARMS), or COLD-PCR (coamplification at lower denaturation temperature PCR) with sequencing. Other specific exemplary methods of screening for Ras mutations include, but are not limited to: blood-based screening methods relying on circulating tumor DNA (ctDNA) (see, for example, Schmiegel et al., Mol. Oncol., Vol. 11, Issue 2, pp. 208-19 (February 2017) (screening for mutations by applying an emulsion digital PCR-based assay for exons 2, 3, and 4 of KRAS and NRAS to circulating cell-free DNA assay)) and tissue-based methods, such as screening for mutations in KRAS and NRAS exons 2, 3, and 4 in tumor tissue samples using Sanger sequencing, massively parallel sequencing (including sequencing methodologies based on pyrosequencing, cyclic reversible termination, semiconductor sequencing, or phospholinked fluorescent nucleotide technologies), or PCR-based assays (including quantitative PCR and digital PCR). The present invention is not limited to any particular method for screening for Ras mutation status. In some embodiments, the sample or subject has been determined to be RAS wild type before staining for EGFR and EGFR ligands is performed. In other embodiments, the sample is stained for EGFR and EGFR ligands regardless of RAS mutation status.

In an embodiment, the EGFR antibody-based therapy is incorporated into a treatment regime for a RAS wild-type subject having a stage III colorectal tumor. Surgical removal of the tumor or a partial colectomy (including removal of nearby lymph nodes) followed by adjuvant chemotherapy and/or radiation therapy is typically performed at this stage, although chemotherapy (optionally in combination with radiation therapy) may be used without surgery for certain patients. Common chemotherapies include fluoropyrimidine-based chemotherapies, optionally in combination with leucovorin and/or alkylating agents (such as oxaliplatin). Non-limiting combination therapies used at this stage include FOLFOX (5-FU, leucovorin, and oxaliplatin) or CapeOx (capecitabine and oxaliplatin). In one specific non-limiting embodiment, a method of treating a stage III colorectal cancer may comprise:
for subjects having (a) the expression pattern of EGFR and one or more of EREG and AREG indicates that the patient is likely to respond to the EGFR antibody-based therapy, and (b) a RAS wild-type status: administering the EGFR antibody-based therapy, optionally in combination fluoropyrimidine-based chemotherapy or a fluoropyrimidine-based combination chemotherapy (such as FOLFOX or CapeOx); or
for subjects wherein either (a) the expression pattern of EGFR and one or more of EREG and AREG indicates that the patient is not likely to respond to the EGFR antibody-based therapy; and/or (b) an activating RAS mutation is present, administering a therapy course that does not comprise the EGFR antibody-based.

In another embodiment, the EGFR antibody-based therapy is incorporated into a treatment regime for a RAS wild-type subject having a stage IV colorectal tumor. Therapeutic regimes for stage IV colorectal tumors typically include surgical removal of the tumor or a partial colectomy (including removal of nearby lymph nodes) and metastases (if possible) and adjuvant or neoadjuvant chemotherapy and/or radiation therapy. Surgical removal of the tumor or a partial colectomy (including removal of nearby lymph nodes) and metastases (if possible), as well as chemotherapy and/or radiation therapy is typically performed at this stage. Common chemotherapies include fluoropyrimidine-based chemotherapies, optionally in combination with leucovorin and/or other chemotherapies and/or targeted therapies. Non-limiting combination therapies used at this stage include:
FOLFOX: leucovorin, 5-FU, and oxaliplatin (ELOXATIN);
FOLFIRI: leucovorin, 5-FU, and irinotecan (CAMPTOSAR);
CapeOX: capecitabine (XELODA) and oxaliplatin;
FOLFOXIRI: leucovorin, 5-FU, oxaliplatin, and irinotecan;
One of the above combinations plus either a drug that targets VEGF (such as bevacizumab [AVASTIN], ziv-aflibercept [ZALTRAP], or ramucirumab [CYRAMZA]), or a drug that targets EGFR (such as cetuximab [Erbitux] or panitumumab [VECTIBIX]);
5-FU and leucovorin, with or without a targeted drug;
Capecitabine, with or without a targeted drug;
Irinotecan, with or without a targeted drug;
Cetuximab alone;
Panitumumab alone;
Regorafenib (STIVARGA) alone; and
Trifluridine and tipiracil (LONSURF),
   In one specific non-limiting embodiment, a method of treating a stage IV colorectal cancer may comprise:
for subjects having (a) the expression pattern of EGFR and one or more of EREG and AREG indicates that the patient is likely to respond to the EGFR antibody-based therapy; and (b) a RAS wild-type status, administering the EGFR antibody-based therapy, optionally in combination with one or more additional therapies selected from the group consisting of FOLFOX, FOLFIRI, CapeOX, FOLFOXIRI, 5-FU and leucovorin, capecitabine, irinotecan, and a drug that targets VEGF (such as bevacizumab, ziv-aflibercept, and ramucirumab); or
for subjects wherein either (a) the expression pattern of EGFR and one or more of EREG and AREG indicates that the patient is not likely to respond to the EGFR antibody-based therapy; and/or (b) an activating RAS mutation is present, administering a therapy course that does not comprise the EGFR antibody-based therapy (such as a drug that targets VEGF, FOLFOX (optionally in combination with a drug that targets VEGF), FOLFIRI (optionally in combination with a drug that targets VEGF), CapeOX (optionally in combination with a drug that targets VEGF), FOLFOXIRI (optionally in combination with a drug that targets VEGF), 5-FU and leucovorin (optionally in combination with a drug that targets VEGF), Capecitabine (optionally in combination with a drug that targets VEGF), Irinotecan (optionally in combination with a drug that targets VEGF), Regorafenib, or Trifluridine and tipiracil (optionally in combination with a drug that targets VEGF)).

### V. EXAMPLES

### Example 1: Colorectal Cancer Samples and Sample Processing

In a study of 57 colorectal cancer cases, eleven 4 μm cuts were obtained for each sample and they were stained in the following order (see Table 8).

TABLE 8

| Cut/Slide | Stain Performed |
|---|---|
| 1 | H & E |
| 2 | EREG/AREG/EGFR Multiplex IHC |
| 3 | AREG DAB IHC |
| 4 | AREG mRNA-ISH |
| 5 | EREG DAB IHC |
| 6 | EREG mRNA-ISH |
| 7 | EGFR DAB IHC |
| 8 | EGFR mRNA-ISH (probe set 1) |
| 9 | EGFR mRNA-ISH (probe set 2) |
| 10 | Actin mRNA-ISH |
| 11 | Sent for qPCR |

Slide 2 utilized a multiplex IHC method performed on a BenchMark ULTRA instrument. The antibodies used included: EGFR (5B7) rabbit antibody clone, EREG (L8) rabbit antibody clone, and AREG (L10) rabbit antibody clone. EGFR was stained with DISCOVERY Yellow, EREG was stained with DISCOVERY Teal, and AREG was stained with DISCOVERY Purple. Since the three primary antibodies were each rabbit antibodies, a sequential multiplex staining method was used, wherein cell conditioning buffer 2 (CC2) and heat were applied to the tissue sections after each round of staining to denature the antibodies and prevent cross reactivity. An example of a protocol for multiplex staining herein may be summarized as follows: Apply deparaffinization buffer; apply antigen retrieval buffer; apply anti-EREG antibody and detection reagents; apply a heat kill step; apply anti-EGFR antibody and detection reagents; apply a heat kill step; and apply anti-AREG antibody and detection reagents. A description of a protocol used for the multiplex staining method in a BenchMark ULTRA (Ventana Medical Systems, Inc.) for this example is shown in Table 9 below. The present invention is not limited to this protocol.

TABLE 9
Triplex Brightfield IHC (BenchMark ULTRA IHC/ISH Staining Module)

```
  1 Enable Mixers
  2 Disable Mixers
  3 [72° C. is the Standard Temperature]
  4 Warmup Slide to [72 Deg C.] from Medium Temperatures (Deparaffinization)
  5 Incubate for 4 Minutes
  6 Apply EZPrep Volume Adjust
  7 Rinse Slide With EZ Prep
  8 Apply EZPrep Volume Adjust
  9 Apply Coverslip
  10 Rinse Slide With EZ Prep
  11 Apply EZPrep Volume Adjust
  12 Apply Coverslip
  13 Enable Mixers
  14 Disable Slide Heater
  15 Pause Point (Landing Zone)
  16 Rinse Slide With EZ Prep
  17 Apply Long Cell Conditioner #1
  18 Apply CC Coverslip Long
  19 Warmup Slide to [100 Deg C.], and Incubate for 4 Minutes (Cell Conditioner #1)
  20 [100° C. is the Standard Temperature]
  21 Incubate for 4 Minutes
  22 Incubate for 8 Minutes
  23 Apply Cell Conditioner #1
  24 Apply CC Medium Coverslip No BB
  25 Incubate for 8 Minutes
  26 Incubate for 8 Minutes
  27 Apply Cell Conditioner #1
  28 Apply CC Medium Coverslip No BB
  29 Incubate for 8 Minutes
  30 Incubate for 8 Minutes
  31 Apply Cell Conditioner #1
  32 Apply CC Medium Coverslip No BB
  33 Incubate for 8 Minutes
  34 Apply Cell Conditioner #1
  35 Apply CC Medium Coverslip No BB
  36 Apply Cell Conditioner #1
  37 Apply CC Medium Coverslip No BB
  38 Apply Cell Conditioner #1
  39 Apply CC Medium Coverslip No BB
  40 Disable Slide Heater
  41 Apply Cell Conditioner #1
  42 Apply CC Medium Coverslip No BB
  43 Rinse Slide With Reaction Buffer
  44 Adjust Slide Volume With Reaction Buffer
  45 Apply Coverslip
  46 Pause Point (Landing Zone)
  47 Warmup Slide to 36 Deg C.
  48 Rinse Slide With Reaction Buffer
  49 Adjust Slide Volume With Reaction Buffer
  50 Apply One Drop of OV PEROX IHBTR, Apply Coverslip, and Incubate for [0 Hr 4 Min]
  51 Rinse Slide With Reaction Buffer
  52 Adjust Slide Volume With Reaction Buffer
  53 Apply Coverslip
  54 [Default Ab Incubation Temp 36 Deg C.]
  55 Warmup Slide to 36 Deg C., and Incubate for 4 Minutes
  56 Rinse Slide With Reaction Buffer
  57 Adjust Slide Volume With Reaction Buffer
  58 Apply One Drop of [PREP KIT 14] (Antibody), Apply Coverslip, and Incubate for [0 Hr 16 Min]
  59 Rinse Slide With Reaction Buffer
  60 Adjust Slide Volume With Reaction Buffer
  61 Apply Coverslip
  62 Rinse Slide With Reaction Buffer
  63 Adjust Slide Volume With Reaction Buffer
  64 Apply Coverslip
  65 Warmup Slide to 36 Deg C.
  66 Rinse Slide With Reaction Buffer
  67 Adjust Slide Volume With Reaction Buffer
  68 Apply Coverslip, One Drop of OV HQ UNIV LINKR, and Incubate for [8 Minutes]
  69 Rinse Slide With Reaction Buffer
  70 Apply 230 ul + VA Reaction Buffer
  71 Apply Coverslip
  72 Rinse Slide With Reaction Buffer
  73 Apply 230 ul + VA Reaction Buffer
  74 Apply Coverslip
  75 Rinse Slide With Reaction Buffer
  76 Adjust Slide Volume With Reaction Buffer
  77 Apply Coverslip, One Drop of OV HRP MULTIMER, and Incubate for [8 Minutes]
  78 [OptiView HQ Linker & OptiView HRP Multimer (Three-Layer)]
  79 Rinse Slide With Reaction Buffer
  80 Apply 230 ul + VA Reaction Buffer
  81 Apply Coverslip
  82 Rinse Slide With Reaction Buffer
  83 Apply 230 ul + VA Reaction Buffer
  84 Apply Coverslip
  85 [Apply TSA-Chromogen first followed by H2O2]
  86 Rinse Slide With Reaction Buffer
  87 Adjust Slide Volume With Reaction Buffer
  88 Apply Two Drops of [PREP KIT 8] (Antibody 3), Apply Coverslip, and Incubate
  for 4 Minutes
  89 Apply One Drop of [PREP KIT 7] (Antibody 4), and Incubate for [0 Hr 32 Min]
  90 Rinse Slide With Reaction Buffer
  91 Adjust Slide Volume With Reaction Buffer
  92 Apply Coverslip
  93 Rinse Slide With Reaction Buffer
  94 Adjust Slide Volume With Reaction Buffer
  95 Apply Coverslip
  96 [Heat Kill w/CC2-Nominal conditions 100 C., 8 min]
  97 Rinse Slide With EZ Prep
  98 Apply Long Cell Conditioner #2
  99 Apply CC Coverslip Long
100 Warmup Slide to 36 Deg C., and Incubate for 4 Minutes
101 Warmup Slide to [90 Deg C.] from All Temperatures (Antibody)
102 Incubate for [8 Minutes] (Antibody)
103 Apply Cell Conditioner #2
104 Apply CC Medium Coverslip No BB
105 Apply Cell Conditioner #2
106 Apply CC Medium Coverslip No BB
107 Warmup Slide to 36 Deg C.
108 Apply Cell Conditioner #2
109 Apply CC Medium Coverslip
110 Rinse Slide With Reaction Buffer
111 Apply 230 ul + VA Reaction Buffer
112 Apply Coverslip
113 Warmup Slide to 36 Deg C.
114 Rinse Slide With Reaction Buffer
115 Adjust Slide Volume With Reaction Buffer
116 Apply Coverslip
117 [Default Ab Incubation Temp 36 Deg C.]
118 Warmup Slide to 36 Deg C., and Incubate for 4 Minutes
119 Rinse Slide With Reaction Buffer
120 Adjust Slide Volume With Reaction Buffer
121 Apply One Drop of [anti-EGFR (5B7)] (Antibody 11), Apply Coverslip, and
Incubate for [0 Hr 32 Min]
122 Rinse Slide With Reaction Buffer
123 Adjust Slide Volume With Reaction Buffer
124 Apply Coverslip
125 Rinse Slide With Reaction Buffer
126 Adjust Slide Volume With Reaction Buffer
127 Apply Coverslip
128 Warmup Slide to 36 Deg C.
129 Rinse Slide With Reaction Buffer
130 Adjust Slide Volume With Reaction Buffer
131 Apply One Drop of [ANTIBODY 30] (Antibody 12), Apply Coverslip, and
Incubate for [0 Hr 8 Min]
132 [Two-Layer Antibody Stack]
133 [Non-OptiView Three-Layer Antibody Stack]
134 Rinse Slide With Reaction Buffer
135 Apply 230 ul + VA Reaction Buffer
136 Apply Coverslip
137 Rinse Slide With Reaction Buffer
138 Apply 230 ul + VA Reaction Buffer
139 Apply Coverslip
140 Rinse Slide With Reaction Buffer
141 Adjust Slide Volume With Reaction Buffer
142 Apply One Drop of [ANTIBODY 31] (Antibody 19), Apply Coverslip, and
Incubate for [8 Minutes]
143 Rinse Slide With Reaction Buffer
144 Apply 230 ul + VA Reaction Buffer
145 Apply Coverslip
146 Rinse Slide With Reaction Buffer
147 Apply 230 ul + VA Reaction Buffer
148 Apply Coverslip
149 [Alkaline phosphatase QM substrate-pH adjust followed by QM]
150 Rinse Slide With SSC
151 Adjust Slide Volume With SSC
152 Apply Coverslip
153 Rinse Slide With SSC
154 Adjust Slide Volume With SSC
155 Apply Coverslip
156 Rinse Slide With SSC
157 Adjust Slide Volume With SSC
158 Apply Two Drops of [PREP KIT 3] (Antibody 15), Apply Coverslip, and
Incubate for 4 Minutes
159 Apply One Drop of [PREP KIT 4] (Antibody 16), and Incubate for [0 Hr 32 Min]
160 Rinse Slide With SSC
161 Adjust Slide Volume With SSC
162 Apply Coverslip
163 Rinse Slide With Reaction Buffer
164 Adjust Slide Volume With Reaction Buffer
165 Apply Coverslip
166 [Heat Kill w/CC2-Nominal conditions 100 C., 8 min]
167 Rinse Slide With EZ Prep
168 Apply Long Cell Conditioner #2
169 Apply CC Coverslip Long
170 Warmup Slide to 36 Deg C., and Incubate for 4 Minutes
171 Warmup Slide to [100 Deg C.] from All Temperatures (Antibody 2)
172 Incubate for [8 Minutes] (Antibody 2)
173 Apply Cell Conditioner #2
174 Apply CC Medium Coverslip No BB
175 Apply Cell Conditioner #2
176 Apply CC Medium Coverslip No BB
177 Warmup Slide to 36 Deg C.
178 Apply Cell Conditioner #2
179 Apply CC Medium Coverslip
180 Rinse Slide With Reaction Buffer
181 Apply 230 ul + VA Reaction Buffer
182 Apply Coverslip
183 Warmup Slide to 36 Deg C.
184 Rinse Slide With Reaction Buffer
185 Adjust Slide Volume With Reaction Buffer
186 Apply Coverslip
187 [Default Ab Incubation Temp 36 Deg C.]
188 Warmup Slide to 36 Deg C., and Incubate for 4 Minutes
189 Rinse Slide With Reaction Buffer
190 Adjust Slide Volume With Reaction Buffer
191 Apply One Drop of [ANTIBODY 14] (Antibody 21), Apply Coverslip, and
Incubate for [0 Hr 16 Min]
192 Rinse Slide With Reaction Buffer
193 Adjust Slide Volume With Reaction Buffer
194 Apply Coverslip
195 Rinse Slide With Reaction Buffer
196 Adjust Slide Volume With Reaction Buffer
197 Apply Coverslip
198 Warmup Slide to 36 Deg C.
199 Rinse Slide With Reaction Buffer
200 Adjust Slide Volume With Reaction Buffer
201 Apply One Drop of [ANTIBODY 33] (Antibody 22), Apply Coverslip, and
Incubate for [0 Hr 8 Min]
202 [Two-Layer Antibody Stack]
203 [Non-OptiView Three-Layer Antibody Stack]
204 Rinse Slide With Reaction Buffer
205 Apply 230 ul + VA Reaction Buffer
206 Apply Coverslip
207 Rinse Slide With Reaction Buffer
208 Apply 230 ul + VA Reaction Buffer
209 Apply Coverslip
210 Adjust Slide Volume With Reaction Buffer
211 Rinse Slide With Reaction Buffer
212 Apply One Drop of [ANTIBODY 34] (Antibody 29), Apply Coverslip, and
Incubate for [8 Minutes]
213 Rinse Slide With Reaction Buffer
214 Apply 230 ul + VA Reaction Buffer
215 Apply Coverslip
216 Rinse Slide With Reaction Buffer
217 Apply 230 ul + VA Reaction Buffer
218 Apply Coverslip
219 [Apply TSA-Click first followed by H2O2]
220 Rinse Slide With Reaction Buffer
221 Adjust Slide Volume With Reaction Buffer
222 Apply One Drop of [DETECTION 18] (Detection #7), Apply Coverslip, and
Incubate for 4 Minutes
223 Apply One Drop of [DETECTION 19] (Detection #8), and Incubate for
[0 Hr 32 Min]
224 Rinse Slide With Reaction Buffer
225 Adjust Slide Volume With Reaction Buffer
226 Apply Coverslip
227 [Apply Click-Chromogen]
228 Rinse Slide With Reaction Buffer
229 Adjust Slide Volume With Reaction Buffer
230 Apply One Drop of [DETECTION 20] (Detection #9), Apply Coverslip, and
Incubate for 0 Hr 16 Min
231 Rinse Slide With Reaction Buffer
232 Adjust Slide Volume With Reaction Buffer
233 Apply Coverslip
234 Rinse Slide With Reaction Buffer
235 Adjust Slide Volume With Reaction Buffer
236 Apply Coverslip
237 Warmup Slide to 36 Deg C.
238 Rinse Slide With Reaction Buffer
239 Adjust Slide Volume With Reaction Buffer
240 Apply One Drop of [HEMATOXYLIN II] (Counterstain), Apply Coverslip, and
Incubate for [4 Minutes]
241 Rinse Slide With Reaction Buffer
242 Adjust Slide Volume With Reaction Buffer
243 Apply Coverslip
244 Rinse Slide With Reaction Buffer
245 Adjust Slide Volume With Reaction Buffer
246 Apply One Drop of [BLUING REAGENT] (Post Counterstain), Apply Coverslip,
and Incubate for [4 Minutes]
247 Rinse Slide With Reaction Buffer
248 Adjust Slide Volume With Reaction Buffer
249 Apply Coverslip
250 Disable Slide Heater
```

Slides 3, 5, and 7 utilized simplex IHC methods performed on a BenchMark XT instrument. Slide 3 featured the AREG (L10) rabbit antibody clone and an OptiView DAB detection kit. Slide 5 featured the EREG (L8) rabbit antibody and an OptiView DAB detection kit. Slide 7 featured the EGFR (5B7) rabbit antibody and an OptiView DAB detection kit.

For image acquisition and analysis, stained slides were scanned on a VENTANA iSCAN HT slide scanner at 20× magnification and HT focus approach. Read-outs combined overall number and density of IHC-positive and negative cells with descriptive statistics of cell-by-cell expression patterns, spatial patterns of positive cells, and co-location of cells between different markers determined after automated alignment of consecutive or close tissue sections.

### Example 2: Correlation of Simplex Assay to qPCR

Slide 11 from each case in Example 1 was sent for qPCR analysis. For statistical analysis, IHC status was correlated to qPCR. Correlation was measured using Spearman's rho. Subsequently LOESS and single segment linear regressions were plotted on the data. The top tertile for either AREG or EREG qPCR was plotted and the point at which it intersected the regression line was determined to be the associated cutoff point for the IHC parameter.

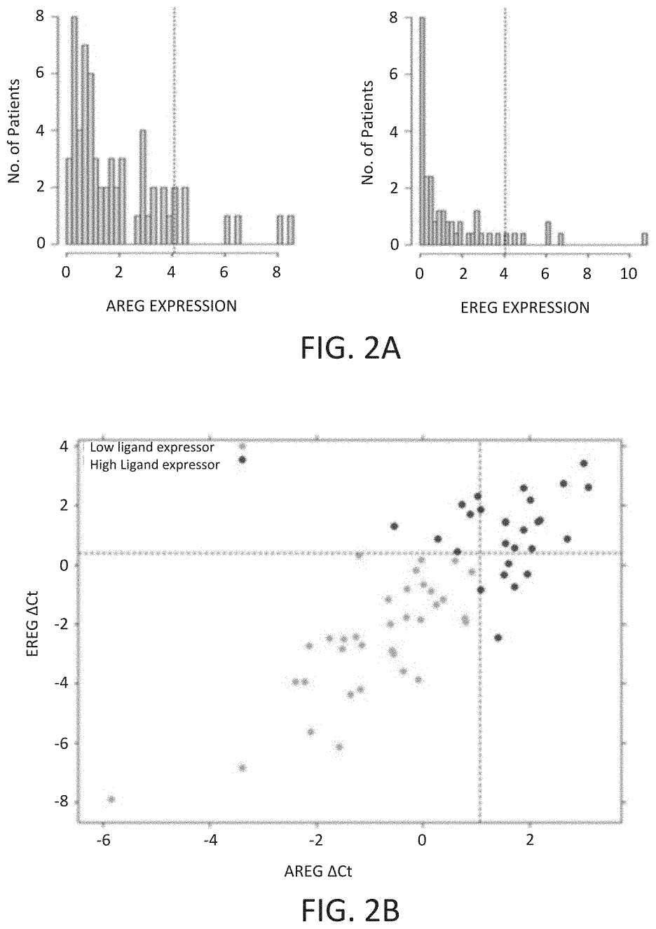

The qPCR results of the samples in Example 1 are comparable to published data. FIG. 2A shows that the distribution of qPCR values is similar to published values, and FIG. 2B shows and the expression of EREG mRNA is closely related to the expression of AREG mRNA.

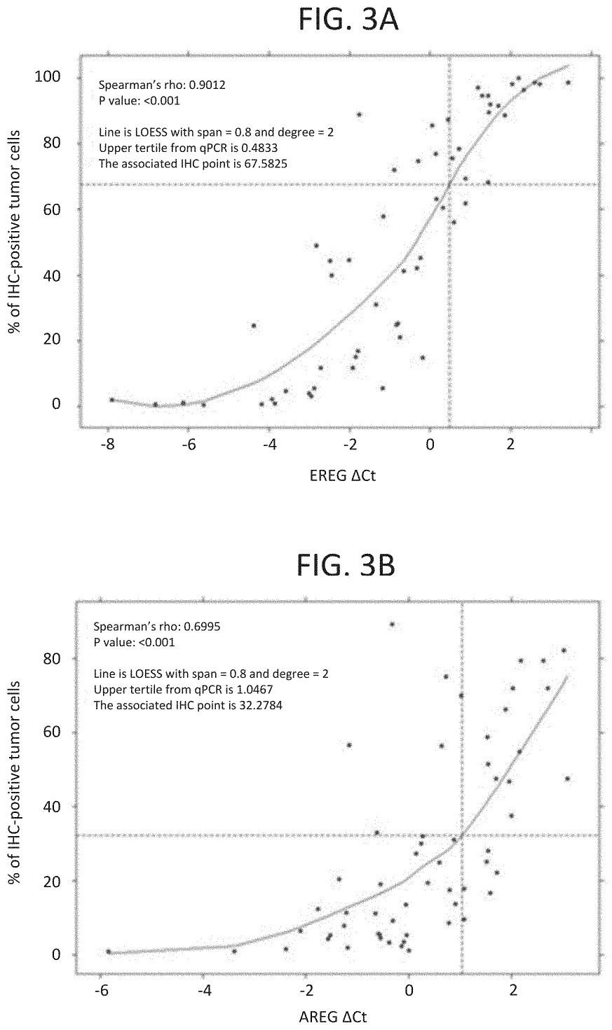

FIG. 3A and FIG. 3B show that percent positivity correlates well to qPCR for both EREG and AREG. FIG. 3A is a scatter plot between percent of tumor cells positive for human EREG protein stained for IHC with qPCR data for the same sample. The scatter plot demonstrates a Spearman's Rho: 0.9012 with a P-value of <0.001 and a LOESS curve having a span of 0.8 and a degree of 2. As can be seen from the LOESS curve, the upper tertile of qPCR expression for EREG has a ΔCT of ≥0.4833, which intersects with a percent positive tumor cells for EREG protein of 67.5825%.

The distribution of data for EREG appears to be a case of comparing two assays with varying dynamic ranges (FIG. 3A). qPCR shows a wider dynamic range, with signal generated below the IHC limit of detection and after IHC is saturated. Similar results were generated for Amphiregulin but the saturation point did not appear to be reached.

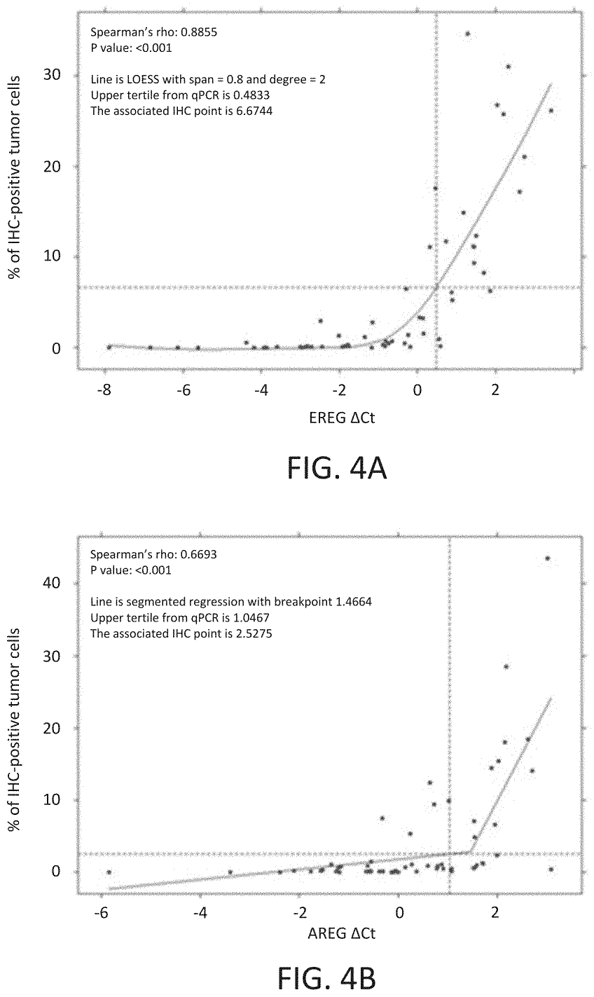

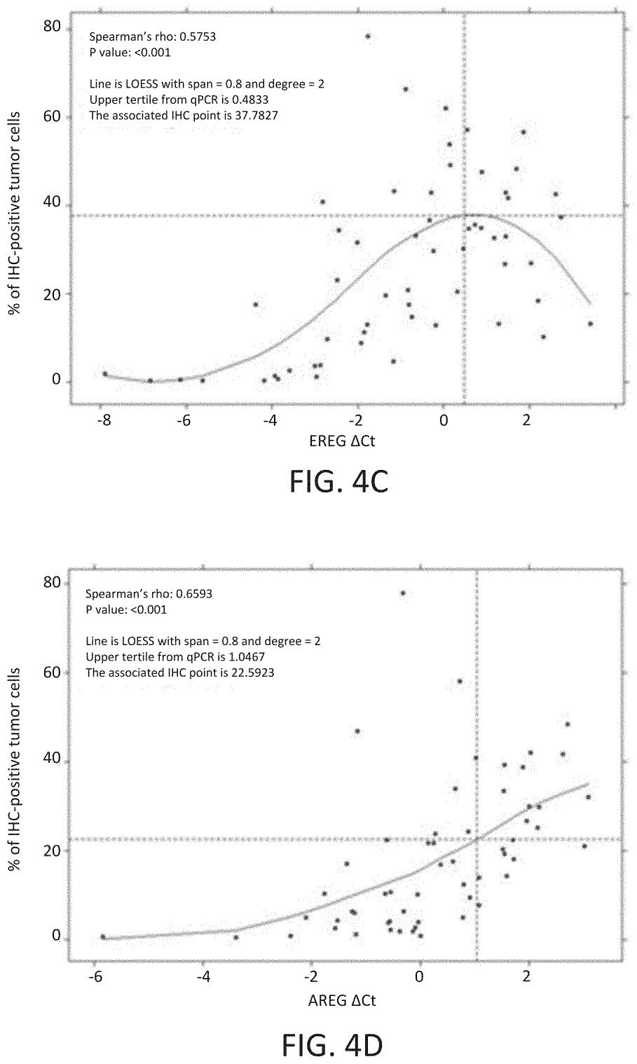

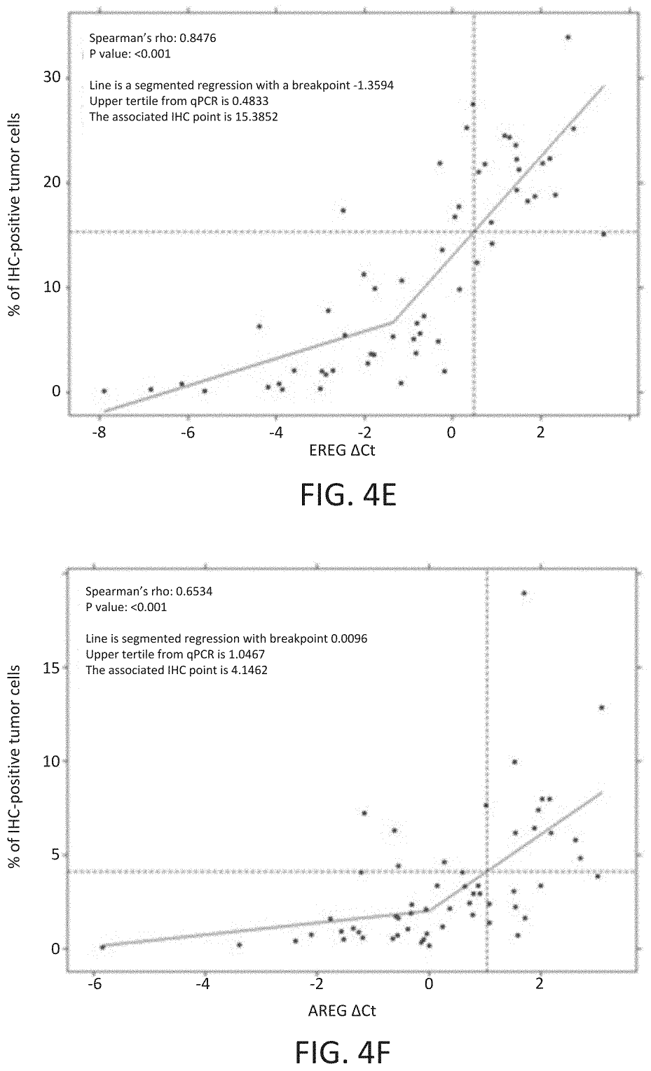

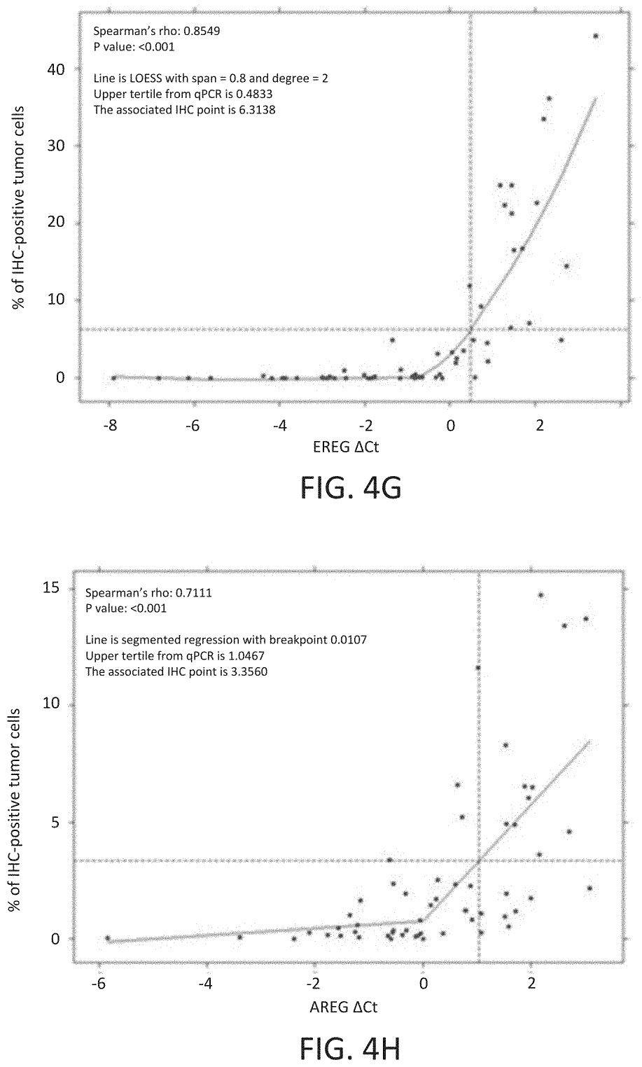

In addition to percent positivity, an unsupervised clustering function as described in U.S. 62/441,068, filed Dec. 30, 2016 (incorporated herein by reference in its entirety) was applied to the images. This assay generated four distinct classifications of marker-positive cells (termed hereafter parameter 1, parameter 2, parameter 3, and parameter 4). It was found that multiple of these parameters are useful and correlate with qPCR. Results are shown at FIG. 4A-4H. Parameter 1 correlates with EREG very well and has a Spearman's Rho of 0.8855, besting %-positivity. The cut point for parameter 1 is 6.6744% for EREG and 2.5275% for AREG (FIG. 4A, FIG. 4B). Parameter 2 correlates less with the qPCR data then other readouts, the spearman's rho was only 0.5753 for EREG and 0.6593 or AREG, but the values still reach significance. This poor association leaves several discordant cases when IHC and qPCR are compared in either marker. Parameters 3 and 4 both have a good correlation with mRNA expression. (FIG. 4E, FIG. 4F, FIG. 4G, FIG. 4H), and P4 gives the best correlation for the AREG IHC (FIG. 4H).

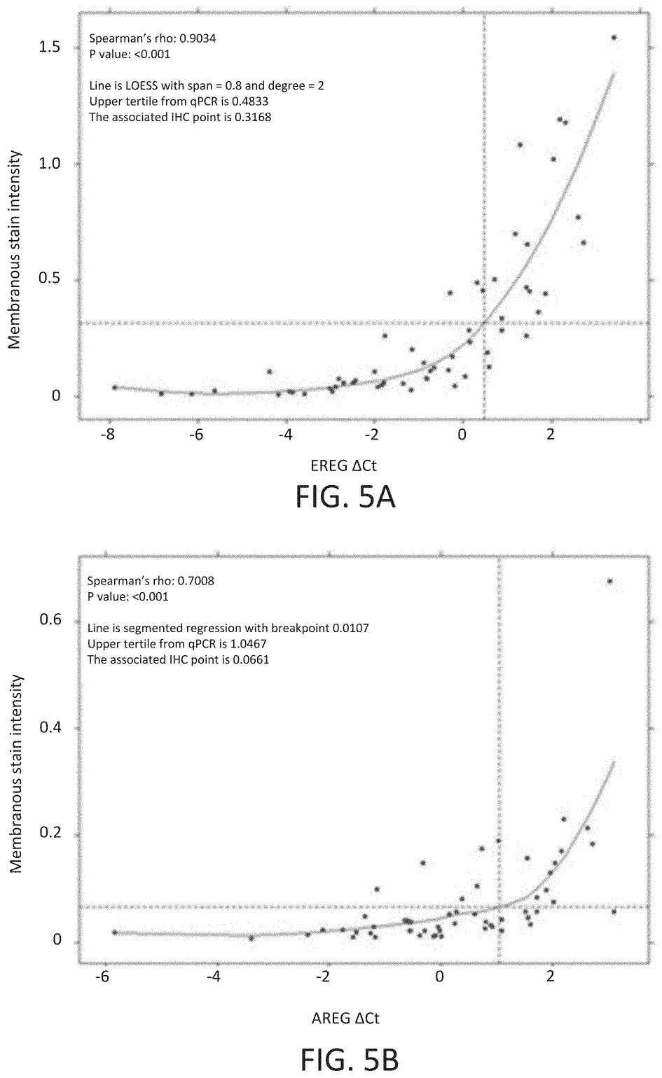

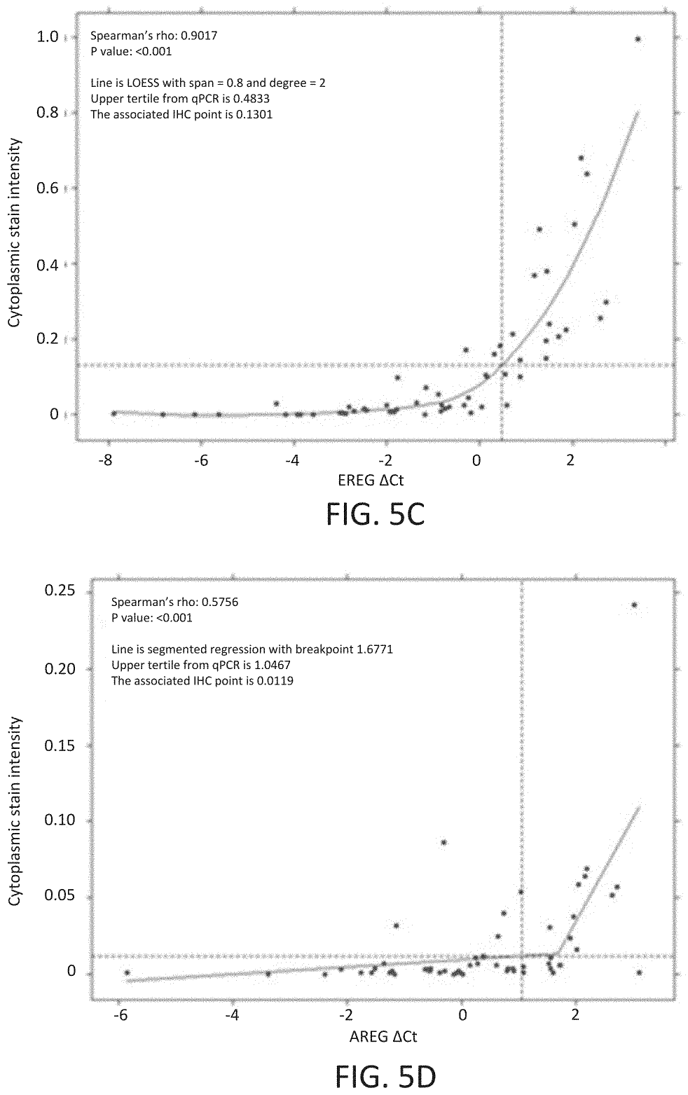

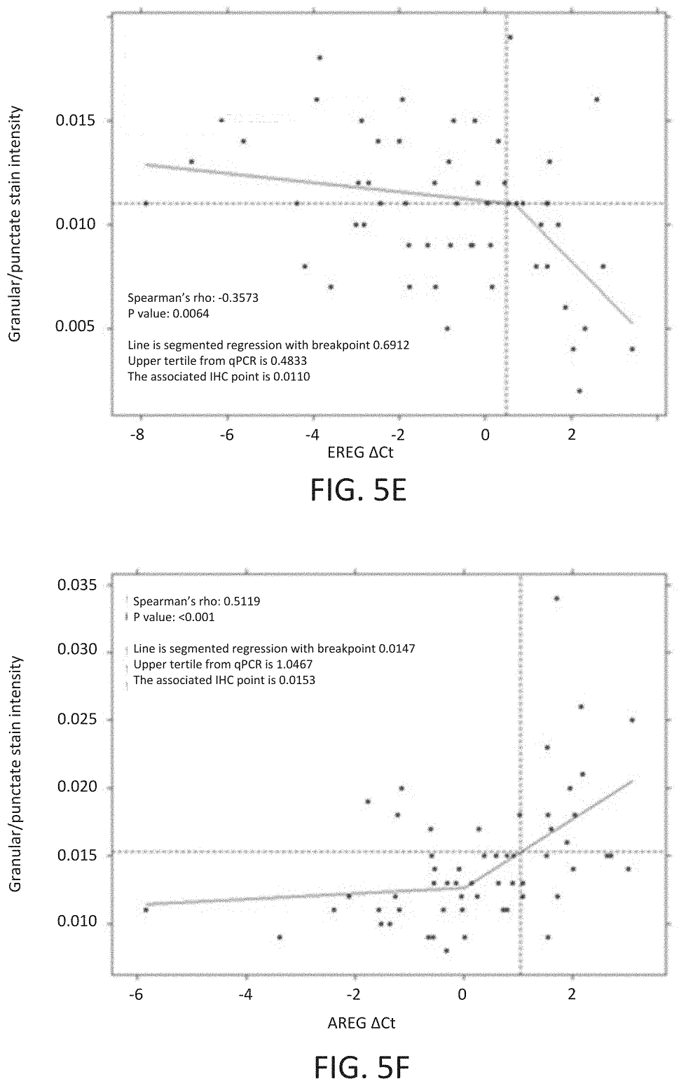

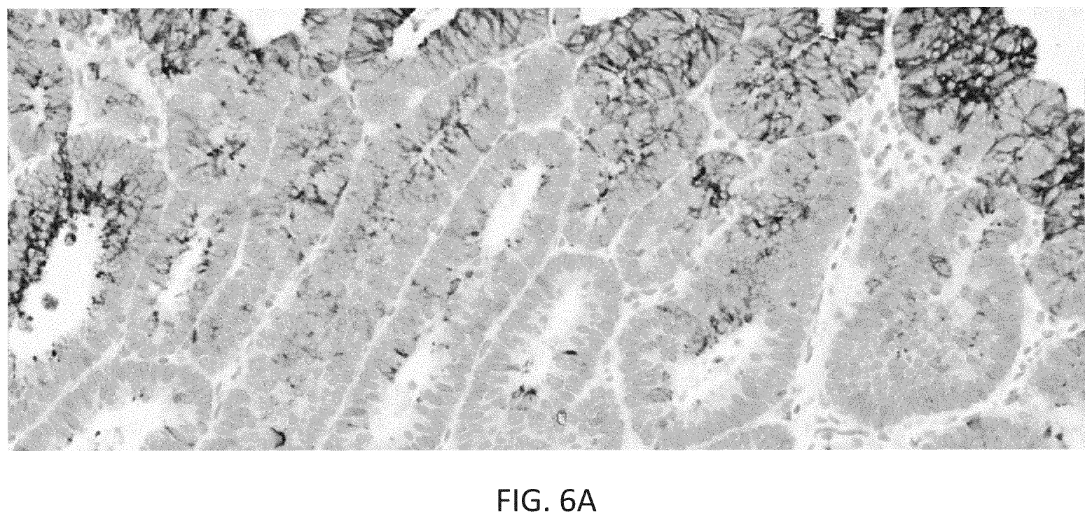

In addition to unbiased parameters, the algorithm also scored the assay for specific sub-cellular localization, including membrane, cytoplasmic, and punctate granules. Automated image analysis determines the overall staining intensity and the staining intensity individual for membrane, cytoplasmic, or punctate patterns on a cell-by-cell basis. In EREG, both membrane and cytoplasmic staining intensity correlate well with mRNA expression. (FIG. 5A, FIG. 5C). Additionally, AREG correlates with membrane staining intensity. (FIG. 5B, FIG. 5D). Note that while the punctate/granular staining pattern is readily apparent and very distinct in both assays, there was very poor correlation with qPCR.

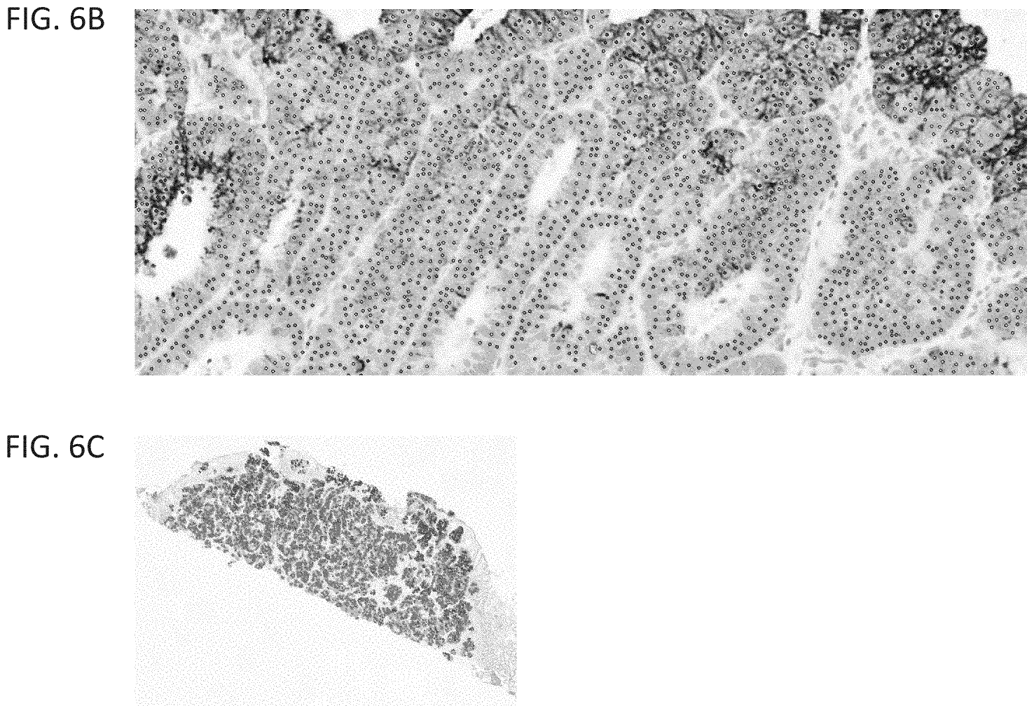

It was then demonstrated that the IHC of EREG and AREG with digital image analysis have similar clinical utility to qPCR analysis of the EGFR ligands. Each computer-generated parameter was correlated to the qPCR values, establishing the IHC cut point. Image analysis results are obtained for all relevant tissue on a slide. High-resolution results for an example field of view (FOV) (see FIG. 6A, FIG. 6B, FIG. 6C) show that the automated analysis identifies every tumor cell and classifies it as being marker-negative (displayed in green and blue) or marker-positive (displayed in yellow, orange, red, and magenta). The number of tumor cells on the whole slide is reported separate for marker-negative and marker-positive cells. The cutoff values for all 11 parameters as well as their Spearman's rho value are set forth below in Table 10:

TABLE 10

| | | | Spearmans′s | | | Spearman′s |
|---|---|---|---|---|---|---|
| | Loess | Segmented | Rho | | Segmented | Rho |
| | AREG | AREG | AREG | Loess EREG | EREG | EREG |
| % IHC | 32.2784 | 35.3634 | 0.6995 | 67.5825 | 67.803 | 0.9012 |
| Positive | | | | | | |
| % P1 | 2.8555 | 2.5375 | 0.6693 | 6.6744 | 7.2163 | 0.8855 |
| % P2 | 22.5923 | 23.0968 | 0.6593 | 37.7827 | 39.0496 | 0.5753 |
| % P3 | 3.8591 | 4.1462 | 0.6534 | 16.8119 | 15.3852 | 0.8476 |
| % P4 | 2.9715 | 3.356 | 0.7111 | 6.3138 | 6.2142 | 0.8549 |
| Area Density | 922.0416 | 954.742 | 0.6432 | 1424.5216 | 1413.39 | 0.7613 |
| Ave | 0.0694 | 0.1201 | 0.6568 | 0.458 | 0.4396 | 0.8427 |
| Intensity | | | | | | |
| Membrane | 0.0661 | 0.0613 | 0.7008 | 0.3168 | 0.3028 | 0.9034 |
| Int | | | | | | |
| Cyto Int | 0.0112 | 0.0119 | 0.5756 | 0.1301 | 0.1011 | 0.9017 |
| Punct Int | 0.0149 | 0.0153 | 0.5119 | 0.0111 | 0.011 | −0.3573 |

Based on the Spearman's Rho value, several EREG parameters have a strong correlation between 0.89 and 0.90. Additionally, the top variables for AREG IHC have a correlation of 0.70 and 0.71.

### Example 3: Correlation of Multiplex Assay to Simplex Assay

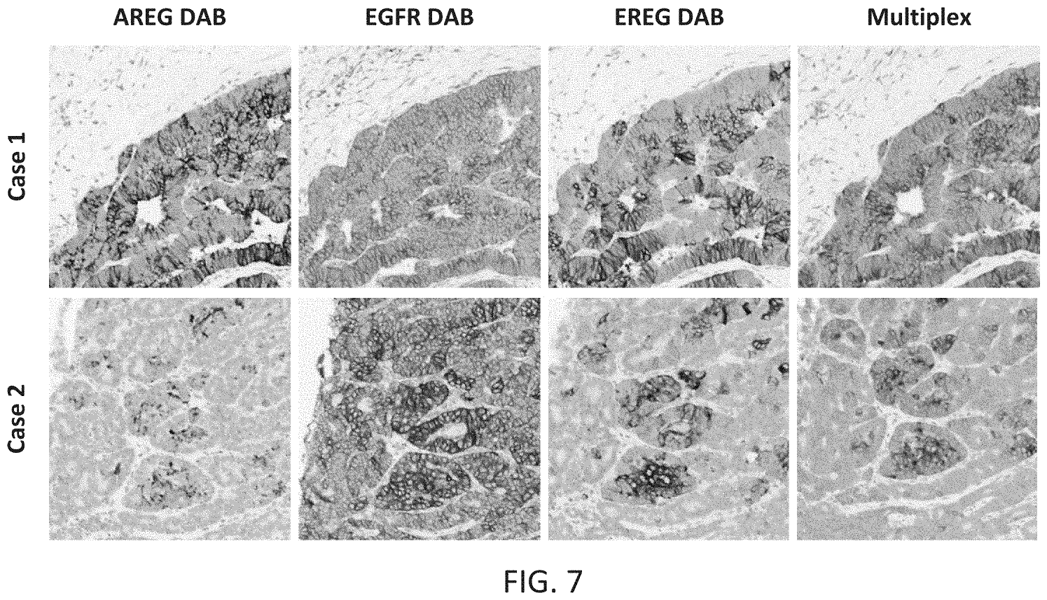

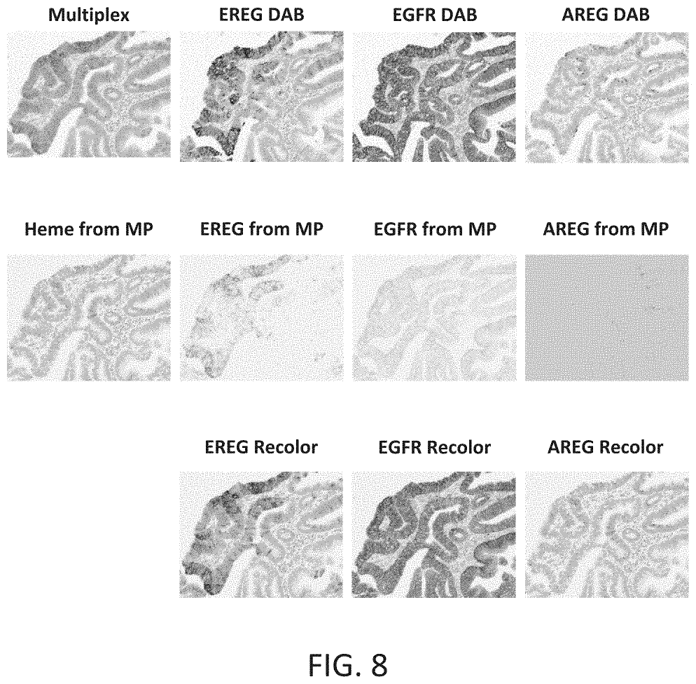

FIG. 7 demonstrates that the colorectal cases of Example 1 could be efficiently stained with a multiplex IHC assay. In the multiplex assay, EGFR was stained with DISCOVERY Yellow, EREG was stained with DISCOVERY Teal, and AREG was stained with tetramethylrhodamine (DISCOVERY Purple). The multiplex assay results (Slide 2) were compared to their equivalent single DAB stains (e.g., Slide 3 for AREG, Slide 5 for EREG, and Slide 7 for EGFR). The first row of FIG. 8 shows that the multiplex staining matches the signals of the corresponding DAB simplex assays. The second row of FIG. 8 shows that the multiplex assay is capable of being deconstructed into its constituent stains using digital image analysis. The third row shows deconstructed channels can be recombined and re-colored in order to create a pseudo-DAB image. FIG. 8 demonstrates that the multiplex assay can provide the same predictive capability as the simplex assay.

1. A method comprising:
(a) contacting a tissue section with a human EGFR protein biomarker specific binding agent and detection reagents sufficient to deposit a first chromogen in proximity to the human EGFR protein biomarker specific binding agent bound to the tissue section;
(b) contacting the tissue section with an AREG protein biomarker specific binding agent and detection reagents sufficient to deposit a second chromogen in proximity to the AREG protein biomarker specific binding agent bound to the tissue section; and
(c) contacting the tissue section with an EREG protein biomarker specific binding agent and detection reagents sufficient to deposit a third chromogen in proximity to the EREG protein biomarker specific binding agent bound to the tissue section;
wherein the first chromogen is deconvolutable from the second chromogen and the third chromogen.

2. The method of claim 1, wherein the biomarker specific binding agents are antibodies or antigen binding fragments thereof.

3. The method of claim 1, wherein the tissue section is a formalin-fixed paraffin embedded (FFPE) tissue section.

4. The method of claim 1, wherein the tissue section is from a colorectal tumor sample.

5. The method of claim 1, wherein the tissue section is from a polyp.

6. The method of claim 1, wherein the tissue section is RAS wild type.

7. The method of claim 1, wherein the tissue section does not comprise a mutation that allows ligand-independent EGFR signaling.

8. The method of claim 1, wherein the tissue section does not comprise RAS proteins with mutations that confer resistance to EGFR monoclonal antibody therapy.

9. The method of claim 1, further comprising visualizing the chromogens using bright-field microscopy.

10. The method of claim 1, wherein antigen retrieval for EGFR, EREG, and AREG are compatible.

11. The method of claim 1, wherein the method allows for determining spatial relationships of EGFR and EGFR ligand expression.

12. The method of claim 1, wherein the second chromogen and the third chromogen are the same.

13. The method of claim 1, wherein the second chromogen is deconvolutable from the third chromogen.

14. A method comprising:
(a) contacting a first tissue section with an EGFR protein specific binding agent and detection reagents sufficient to deposit a first chromogen in proximity to the EGFR protein specific binding agent bound to the first tissue section;
(b) contacting a second tissue section with an AREG protein specific binding agent and detection reagents sufficient to deposit a second chromogen in proximity to the AREG protein specific binding agent bound to the tissue section; and
(c) contacting a third tissue section with an EREG protein specific binding agent and detection reagents sufficient to deposit a third chromogen in proximity to the EREG protein specific binding agent bound to the tissue section;
wherein the first tissue section, the second tissue section, and the third tissue section are serial sections.

15. The method of claim 14, wherein the specific binding agents are antibodies or antigen binding fragments thereof.

16. The method of claim 14, wherein the tissue section is a formal-fixed paraffin embedded (FFPE) tissue section.

17. The method of claim 14, wherein the tissue section is from a colorectal tumor sample.

18. The method of claim 14, wherein the tissue section is from a polyp.

19. The method of claim 14, wherein the tissue section is RAS wild type.

20. The method of claim 14, wherein the tissue section does not comprise a mutation that allows ligand-independent EGFR signaling.

21. The method of claim 14, wherein the serial sections are aligned to match cells.

22. A method comprising:
a. annotating a region of interest (ROI) on a digital image of a tissue section of a colorectal tumor histochemically stained for EGFR, AREG, and EREG;
b. detecting EGFR in at least a portion of the ROI;
c. obtaining an object metric for EGFR within the ROI;
d. detecting AREG, EREG, or both AREG and EREG in at least one portion of the ROI;
e. obtaining an object metric for AREG, EREG, or both AREG and EREG within the ROI; and
f. obtaining a feature vector from the object metric, and applying the feature vector to a scoring function to calculate a score.

23. The method of claim 22, wherein the object metric is selected from Table 2.

24. The method of claim 22, wherein the scoring function is a Cox proportional hazard model.

25. The method of claim 22, wherein the ROI is identified in a digital image of a first serial section of the test sample, wherein the first serial section is stained with hematoxylin and eosin, and wherein the ROI is automatically registered to a digital image of at least a second serial section of the test sample, wherein the second serial section is stained with EGFR, AREG, and EREG.

26. The method of claim 22, wherein the ROI is identified in a digital image of a first serial section of the test sample, wherein the first serial section is stained with hematoxylin and eosin, and wherein the ROI is automatically registered to a digital image of at least a second serial section, a third serial section of the test sample, and a fourth serial section of the test sample, wherein the second serial section is stained with EGFR, the third serial section is stained with EGFR, and the fourth serial section is stained with EREG.

27. A computer-implemented method comprising causing a computer processor to execute a set of computer-executable functions stored on a memory, the set of computer-executable functions comprising:
a. obtaining a digital image of at least one tissue section of a tissue sample, the tissue section is histochemically stained for EGFR and one or more EGFR ligands;
b. annotating one or more regions of interest (ROI) in the digital image; and
c. calculating an object metric of the ROI according to Table 2;
d. calculating a feature vector for the object metric of the ROI; and
e. applying a scoring function to the feature vector, wherein the scoring function generates a score.

28. A method of developing a scoring function, the method comprising:
(a) obtaining one or more a digital images of one or more serial sections of a tumor tissue sample, the tumor tissue sample being part of a cohort of tumor tissue samples from a plurality of subjects with known outcomes, wherein at least a portion of the one or more serial sections of the tumor tissue sample are stained for EGFR and one or more EGFR ligands;
(b) annotating one or more regions of interest (ROI) in the digital images;
(c) generating a feature vector comprising:
object metrics for EGFR and the one or more EGFR ligands according to Table 2; and
outcome data for the subject from which the tumor tissue sample was derived;
(d) repeating (a)-(c) for each tumor tissue sample of the cohort to obtain a plurality of feature vectors, each feature vector of the plurality associated with an individual subject; and
(e) modeling the scoring function by applying a scoring function to the plurality of feature vectors.

29. The method of claim 28, wherein the scoring function is a Cox proportional hazard model.

30. The method of claim 28, comprising applying one or more stratification cutoffs based on the scoring function.

31. The method of claim 30, wherein the one or more stratification cutoffs comprise a cutoff between a likely to respond to anti-EGFR therapy category and an unlikely to respond to anti-EGFR therapy category.

32. A workflow method comprising:
(a) preparing a set of serial tissue sections from a tumor of a patient;
(b) identifying Ras mutation status in a serial tissue section or other portion of the tumor or the patient;
(c) histochemically staining a serial tissue section from the set of serial tissue sections for EGFR and one or more EGFR ligands according to claim 1;
(d) acquiring a digital image of the stained tissue section;
(e) identifying a region of interest (ROI) in the stained tissue section and calculating an object metric in the ROI to obtain a score;
(f) comparing the score to a threshold to stratify the patient into a first category if the score is beyond the threshold and the tumor is Ras mutation negative, or a second category if the score is below the threshold and the tumor or Ras mutation positive.

33. The method of claim 32 further comprising administering to the patient an anti-EGFR therapy if the patient is stratified into the “likely to respond to an anti-EGFR therapy” category.

34. The method of claim 33, wherein the anti-EGFR therapy is effective to disrupt ligand-dependent signaling through EGFR.

35. The method of claim 33, wherein the anti-EGFR therapy is an anti-EGFR monoclonal antibody.

36. The method of claim 32, wherein step (b) for identifying Ras mutation status is performed before step (c) for histochemical staining of the serial tissue section for EGFR and one or more EGFR ligands.

37. The method of claim 32, wherein step (b) for identifying Ras mutation status is performed in parallel with step (c) for histochemical staining of the serial tissue section for EGFR and one or more EGFR ligands.

38. The method of claim 32, wherein step (b) for identifying Ras mutation status is performed after step (c) for histochemical staining of the serial tissue section for EGFR and one or more EGFR ligands.


SEQUENCE LISTING (6 sequences)

SEQ ID NO: 1 - protein, 1210 aa, Homo sapiens [sequence omitted]

SEQ ID NO: 2 - protein, 169 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 3 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 4 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 5 - protein, 189 aa, Homo sapiens [sequence omitted]

SEQ ID NO: 6 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM